SEQUENCE LISTING (101 sequences)

SEQ ID NO: 1 - protein, 207 aa, synthetic construct
  REGION 1..207: note = I53-34A
  VARIANT 1: note = optionally absent
MEGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 2 - protein, 156 aa, synthetic construct
  REGION 1..156: note = I53-34B
  VARIANT 1: note = optionally absent
MTKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 3 - protein, 156 aa, synthetic construct
  REGION 1..156: note = I53-40A
  VARIANT 1: note = optionally absent
MTKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 4 - protein, 209 aa, synthetic construct
  REGION 1..209: note = I53-40B
  VARIANT 1: note = optionally absent
MSTINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 5 - protein, 114 aa, synthetic construct
  REGION 1..114: note = I53-47A
  VARIANT 1: note = optionally absent
MPIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 6 - protein, 157 aa, synthetic construct
  REGION 1..157: note = I53-47B
  VARIANT 1: note = optionally absent
MNQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 7 - protein, 205 aa, synthetic construct
  REGION 1..205: note = I53-50A
  VARIANT 1: note = optionally absent
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 8 - protein, 157 aa, synthetic construct
  REGION 1..157: note = I53-50B
  VARIANT 1: note = optionally absent
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 9 - protein, 177 aa, synthetic construct
  REGION 1..177: note = I53-51A
  VARIANT 1: note = optionally absent
MFTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 10 - protein, 157 aa, synthetic construct
  REGION 1..157: note = I53-51B
  VARIANT 1: note = optionally absent
MNQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 11 - protein, 201 aa, synthetic construct
  REGION 1..201: note = I52-03A
  VARIANT 1: note = optionally absent
MGHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 12 - protein, 237 aa, synthetic construct
  REGION 1..237: note = I52-03B
  VARIANT 1: note = optionally absent
MYEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 13 - protein, 138 aa, synthetic construct
  REGION 1..138: note = I52-32A
  VARIANT 1: note = optionally absent
MGMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 14 - protein, 154 aa, synthetic construct
  REGION 1..154: note = I52-32B
  VARIANT 1: note = optionally absent
MKYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 15 - protein, 164 aa, synthetic construct
  REGION 1..164: note = I52-33A
  VARIANT 1: note = optionally absent
MAVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 175 aa, synthetic construct
  REGION 1..175: note = I52-33B
  VARIANT 1: note = optionally absent
MGANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 17 - protein, 208 aa, synthetic construct
  REGION 1..208: note = I32-06A
  VARIANT 1: note = optionally absent
MTDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 18 - protein, 128 aa, synthetic construct
  REGION 1..128: note = I32-06B
  VARIANT 1: note = optionally absent
MITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 19 - protein, 235 aa, synthetic construct
  REGION 1..235: note = I32-19A
  VARIANT 1: note = optionally absent
MGSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 20 - protein, 162 aa, synthetic construct
  REGION 1..162: note = I32-19B
  VARIANT 1: note = optionally absent
MSGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 21 - protein, 157 aa, synthetic construct
  REGION 1..157: note = I32-28A
  VARIANT 1: note = optionally absent
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 22 - protein, 157 aa, synthetic construct
  REGION 1..157: note = I32-28B
  VARIANT 1: note = optionally absent
MILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 23 - protein, 156 aa, synthetic construct
  REGION 1..156: note = I53-40A.1
  VARIANT 1: note = optionally absent
MTKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 24 - protein, 209 aa, synthetic construct
  REGION 1..209: note = I53-40B.1
  VARIANT 1: note = optionally absent
MDDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 25 - protein, 114 aa, synthetic construct
  REGION 1..114: note = I53-47A.1
  VARIANT 1: note = optionally absent
MPIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 26 - protein, 114 aa, synthetic construct
  REGION 1..114: note = I53-47A.1NegT2
  VARIANT 1: note = optionally absent
MPIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 27 - protein, 157 aa, synthetic construct
  REGION 1..157: note = I53-47B.1
  VARIANT 1: note = optionally absent
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 28 - protein, 157 aa, synthetic construct
  REGION 1..157: note = I53-47B.1NegT2
  VARIANT 1: note = optionally absent
MNQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 29 - protein, 205 aa, synthetic construct
  REGION 1..205: note = I53-50A.1
  VARIANT 1: note = optionally absent
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 30 - protein, 205 aa, synthetic construct
  REGION 1..205: note = I53-50A.1NegT2
  VARIANT 1: note = optionally absent
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 31 - protein, 205 aa, synthetic construct
  REGION 1..205: note = I53-50A.1PosT1
  VARIANT 1: note = optionally absent
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 32 - protein, 157 aa, synthetic construct
  REGION 1..157: note = I53-50B.1
  VARIANT 1: note = optionally absent
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 33 - protein, 157 aa, synthetic construct
  REGION 1..157: note = I53-50B.1NegT2
  VARIANT 1: note = optionally absent
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 34 - protein, 157 aa, synthetic construct
  REGION 1..157: note = I53-50B.4PosT1
  VARIANT 1: note = optionally absent
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 35 - protein, 156 aa, synthetic construct
  REGION 1..156: note = I53-40 A genus
  VARIANT 1: note = optionally absent
  VARIANT 70: note = Xaa is A or K
MTKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 209 aa, synthetic construct
  REGION 1..209: note = I53-40 B genus
  VARIANT 1: note = optionally absent
  VARIANT 2: note = Xaa is S or D
  VARIANT 3: note = Xaa is T or D
  VARIANT 10: note = Xaa is A or R
  VARIANT 85: note = Xaa is T or D
  VARIANT 119: note = Xaa is A or Q
  VARIANT 163: note = Xaa is S or D
  VARIANT 189: note = Xaa is T or R
MXXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 37 - protein, 114 aa, synthetic construct
  REGION 1..114: note = I53-47A genus
  VARIANT 1: note = optionally absent
  VARIANT 13: note = Xaa is T or D
  VARIANT 33: note = Xaa is K or E
  VARIANT 71: note = Xaa is S or D
  VARIANT 74: note = Xaa is R or E
  VARIANT 101: note = Xaa is N or D
  VARIANT 103: note = Xaa is N or D
MPIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 38 - protein, 157 aa, synthetic construct
  REGION 1..157: note = I53-47B genus
  VARIANT 1: note = optionally absent
  VARIANT 9: note = Xaa is Y or H
  VARIANT 82: note = Xaa is N or D
  VARIANT 87: note = Xaa is R or D
  VARIANT 105: note = Xaa is S or D
  VARIANT 119: note = Xaa is R or E
  VARIANT 121: note = Xaa is R or E
  VARIANT 124: note = Xaa is A or D
  VARIANT 126: note = Xaa is H or D
  VARIANT 128: note = Xaa is R or E
  VARIANT 150: note = Xaa is A or N
MNQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 39 - protein, 205 aa, synthetic construct
  REGION 1..205: note = I53-50A genus
  VARIANT 1: note = optionally absent
  VARIANT 126: note = Xaa is T or D
  VARIANT 139: note = Xaa is Q or E
  VARIANT 142: note = Xaa is K or E
  VARIANT 160: note = Xaa is N or D
  VARIANT 163: note = Xaa is N or D
  VARIANT 166: note = Xaa is E or K
  VARIANT 169: note = Xaa is D or K
  VARIANT 179: note = Xaa is S, D or K
  VARIANT 183: note = Xaa is K or E
  VARIANT 185: note = Xaa is T, D, or K
  VARIANT 192: note = Xaa is D or K
  VARIANT 195: note = Xaa is A, E, or K
  VARIANT 198: note = Xaa is E or K
  VARIANT 199: note = Xaa is E or K
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 40 - protein, 157 aa, synthetic construct
  REGION 1..157: note = I53-50B genus
  VARIANT 1: note = optionally absent
  VARIANT 9: note = Xaa is Y or H
  VARIANT 38: note = Xaa is A or R
  VARIANT 82: note = Xaa is N or D
  VARIANT 87: note = Xaa is R or D
  VARIANT 97: note = Xaa is N or D
  VARIANT 105: note = Xaa is S, N, or D
  VARIANT 119: note = Xaa is R, E, or N
  VARIANT 121: note = Xaa is R, E, or D
  VARIANT 122: note = Xaa is K or D
  VARIANT 124: note = Xaa is K or D
  VARIANT 126: note = Xaa is H or D
MNQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 41 - protein, 159 aa, synthetic construct
  REGION 1..159: note = T32-28A
  VARIANT 1: note = optionally absent
MGEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 42 - protein, 184 aa, synthetic construct
  REGION 1..184: note = T32-28B
  VARIANT 1: note = optionally absent
MSQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 43 - protein, 103 aa, synthetic construct
  REGION 1..103: note = T33-09A
  VARIANT 1: note = optionally absent
MEEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 44 - protein, 122 aa, synthetic construct
  REGION 1..122: note = T33-09B
  VARIANT 1: note = optionally absent
MVRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 45 - protein, 177 aa, synthetic construct
  REGION 1..177: note = T33-15A
  VARIANT 1: note = optionally absent
MSKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 46 - protein, 122 aa, synthetic construct
  REGION 1..122: note = T33-15B
  VARIANT 1: note = optionally absent
MVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 47 - protein, 172 aa, synthetic construct
  REGION 1..172: note = T33-21A
  VARIANT 1: note = optionally absent
MRITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 48 - protein, 123 aa, synthetic construct
  REGION 1..123: note = T33-21B
  VARIANT 1: note = optionally absent
MPHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 49 - protein, 158 aa, synthetic construct
  REGION 1..158: note = T33-28A
  VARIANT 1: note = optionally absent
MESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 50 - protein, 113 aa, synthetic construct
  REGION 1..113: note = T33-28B
  VARIANT 1: note = optionally absent
MPVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 51 - protein, 103 aa, synthetic construct
  REGION 1..103: note = T33-31A
  VARIANT 1: note = optionally absent
MEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 52 - protein, 7 aa, synthetic construct
  REGION 1..7: note = Synthetic peptide
QNITEEF

SEQ ID NO: 53 - protein, 513 aa, synthetic construct
  REGION 1..513: note = DS-Cav1
  VARIANT 1..25: note = optionally absent
  VARIANT 508..513: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 54 - protein, 27 aa, synthetic construct
  REGION 1..27: note = T4 fibritin foldon domain
GYIPEAPRDGQAYVRKDGEWVLLSTFL

SEQ ID NO: 55 - protein, 15 aa, synthetic construct
  REGION 1..15: note = Linker
GSGGSGSGSGGSGSG

SEQ ID NO: 56 - protein, 8 aa, synthetic construct
  REGION 1..8: note = Linker
GGSGGSGS

SEQ ID NO: 57 - protein, 8 aa, synthetic construct
  REGION 1..8: note = Linker
GSGGSGSG

SEQ ID NO: 58 - protein, 11 aa, synthetic construct
  REGION 1..11: note = Helical extension
EKAAKAEEAAR

SEQ ID NO: 59 - protein, 25 aa, synthetic construct
  REGION 1..25: note = Signal peptide
MELLILKANAITTILTAVTFCFASG

SEQ ID NO: 60 - protein, 540 aa, synthetic construct
  REGION 1..540: note = DS-Cav1-foldon
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFL

SEQ ID NO: 61 - protein, 474 aa, synthetic construct
  REGION 1..474: note = sc9-10 DS-Cav1 A149C Y458C
  VARIANT 1..25: note = optionally absent
  VARIANT 468..474: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELL

SEQ ID NO: 62 - protein, 474 aa, synthetic construct
  REGION 1..474: note = sc9-10 DS-Cav1 A149C Y458C S46G K465Q S215P E92D
  VARIANT 1..25: note = optionally absent
  VARIANT 468..474: note = optionally absent
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELL

SEQ ID NO: 63 - protein, 522 aa, synthetic construct
  REGION 1..522: note = SC-DM (N67I, S215P)
  VARIANT 1..25: note = optionally absent
  VARIANT 486..522: note = optionally absent
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 64 - protein, 522 aa, synthetic construct
  REGION 1..522: note = SC-TM (N67I, S215P, and E487Q)
  VARIANT 1..25: note = optionally absent
  VARIANT 486..522: note = optionally absent
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDQFDASISQVNEKINQSLAFIRKSDELLSAIGGYIPEAPRDGQAYVRKDGEWVLLSTFL

SEQ ID NO: 65 - protein, 490 aa, synthetic construct
  REGION 1..490: note = HMPV F protein, strain CAN97-83 (A2)
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTG

SEQ ID NO: 66 - protein, 490 aa, synthetic construct
  REGION 1..490: note = HMPVF with A113C, A339C, T160F, I177L
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTAGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTG

SEQ ID NO: 67 - protein, 490 aa, synthetic construct
  REGION 1..490: note = HMPV- F with A113C, A120C, A339C, T160F, I177L, and Q426C
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTCGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYCLSKVEGEQHVIKGRPVSSSFDPIKFPEDQFNVALDQVFENIENSQALVDQSNRILSSAEKGNTG

SEQ ID NO: 68 - protein, 542 aa, synthetic construct
  REGION 1..542: note = 115-BV (A185P)
  VARIANT 1..18: note = optionally absent
  VARIANT 490..542: note = optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGGIEGRHHHHHH

SEQ ID NO: 69 - protein, 646 aa, synthetic construct
  REGION 1..646: note = DS-Cav1-foldon-T33-31A
  VARIANT 1..26: note = optionally absent
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 70 - protein, 619 aa, synthetic construct
  REGION 1..619: note = DS-Cav1-T33-31A
  VARIANT 1..25: note = optionally absent
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 71 - protein, 665 aa, synthetic construct
  REGION 1..665: note = DS-Cav1-foldon-T33-15B
  VARIANT 1..25: note = optionally absent
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 72 - protein, 638 aa, synthetic construct
  REGION 1..638: note = DS-Cav1-T33-15B
  VARIANT 1..25: note = optionally absent
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 73 - protein, 769 aa, synthetic construct
  REGION 1..769: note = DS-Cav1-foldon-I53-50A
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 74 - protein, 730 aa, synthetic construct
  REGION 1..730: note = DS-Cav1-I53-50A
  VARIANT 1..25: note = optionally absent
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 75 - protein, 676 aa, synthetic construct
  REGION 1..676: note = DS-Cav1-I32-28A
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSGGSGSDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 76 - protein, 730 aa, synthetic construct
  REGION 1..730: note = DS-Cav1-8GS-HelExt-I53-50A (F10)
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 77 - protein, 764 aa, synthetic construct
  REGION 1..764: note = DS-Cav1-foldon-15GS-HelExt-I53-50A (F14)
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 78 - protein, 774 aa, synthetic construct
  REGION 1..774: note = HMPV F wt_CAN97-83 strain-I53-50A
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHSGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 79 - protein, 725 aa, synthetic construct
  REGION 1..725: note = HMPV F A113C_A339C_T160F_I177L-I53-50A
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTAGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 80 - protein, 725 aa, synthetic construct
  REGION 1..725: note = HMPV F A113C_A339C_T160F_I177L_A120C, Q426C - I53-50A
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTCGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYCLSKVEGEQHVIKGRPVSSSFDPIKFPEDQFNVALDQVFENIENSQALVDQSNRILSSAEKGNTGGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 81 - protein, 703 aa, synthetic construct
  REGION 1..703: note = sc-DS2-I53-50A
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSCIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 82 - protein, 742 aa, synthetic construct
  REGION 1..742: note = tc-DS2-I53-50A
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSCIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 83 - protein, 734 aa, synthetic construct
  REGION 1..734: note = DS-Cav1-12GS-HelExt-I53-50A (F11)
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 84 - protein, 738 aa, synthetic construct
  REGION 1..738: note = DS-Cav1-16GS-HelExt-I53-50A (F12)
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGSGGSGSGGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 85 - protein, 759 aa, synthetic construct
  REGION 1..759: note = DS-Cav1-foldon-10GS-HelExt-I53-50A (F13)
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 86 - protein, 769 aa, synthetic construct
  REGION 1..769: note = DS-Cav1-foldon-20GS-HelExt-I53-50A (F15)
  VARIANT 1..25: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGGSGSGGSSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 87 - protein, 736 aa, synthetic construct
  REGION 1..736: note = sc9-10 DS-Cav1 A149C Y458C-foldon-I53-50A embodiment
  VARIANT 1..25: note = optionally absent
  VARIANT 469..474: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 88 - protein, 697 aa, synthetic construct
  REGION 1..697: note = sc9-10 DS-Cav1 A149C Y458C - F10 embodiment
  VARIANT 1..25: note = optionally absent
  VARIANT 469..474: note = optionally absent
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 89 - protein, 736 aa, synthetic construct
  REGION 1..736: note = sc9-10 DS-Cav1 A149C Y458C S46G K465Q S215P E92D -foldon-I53-50A embodiment
  VARIANT 1..25: note = optionally absent
  VARIANT 469..474: note = optionally absent
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 90 - protein, 697 aa, synthetic construct
  REGION 1..697: note = sc9-10 DS-Cav1 A149C Y458C S46G K465Q S215P E92D - F10 embodiment
  VARIANT 1..25: note = optionally absent
  VARIANT 469..474: note = optionally absent
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 91 - protein, 753 aa, synthetic construct
  REGION 1..753: note = SC-DM (N67I, S215P) - foldon-I53-50A embodiment
  VARIANT 1..25: note = optionally absent
  VARIANT 486..491: note = optionally absent
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 92 - protein, 714 aa, synthetic construct
  REGION 1..714: note = SC-DM (N67I, S215P) - F10 embodiment
  VARIANT 1..25: note = optionally absent
  VARIANT 486..491: note = optionally absent
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 93 - protein, 753 aa, synthetic construct
  REGION 1..753: note = SC-TM (N67I, S215P, and E487Q) - foldon-I53-50A embodiment
  VARIANT 1..25: note = optionally absent
  VARIANT 486..491: note = optionally absent
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDQFDASISQVNEKINQSLAFIRKSDELLGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 94 - protein, 714 aa, synthetic construct
  REGION 1..714: note = SC-TM (N67I, S215P, and E487Q)-I53-50A - F10 embodiment
  VARIANT 1..25: note = optionally absent
  VARIANT 486..491: note = optionally absent
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDQFDASISQVNEKINQSLAFIRKSDELLGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 95 - protein, 752 aa, synthetic construct
  REGION 1..752: note = HMPV-F with A113C, A339C, T160F, I177L - foldon-I53-50A embodiment
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTAGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 96 - protein, 713 aa, synthetic construct
  REGION 1..713: note = HMPV-F with A113C, A339C, T160F, I177L-I53-50A F10 embodiment
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTAGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 97 - protein, 752 aa, synthetic construct
  REGION 1..752: note = HMPV-F with A113C, A120C, A339C, T160F, I177L, and Q426C - foldon-I53-50A embodiment
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTCGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYCLSKVEGEQHVIKGRPVSSSFDPIKFPEDQFNVALDQVFENIENSQALVDQSNRILSSAEKGNTGGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 98 - protein, 713 aa, synthetic construct
  REGION 1..713: note = HMPV-F with A113C, A120C, A339C, T160F, I177L, and Q426C - F10 embodiment
  VARIANT 1..18: note = optionally absent
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVCTAAAVTCGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYCLSKVEGEQHVIKGRPVSSSFDPIKFPEDQFNVALDQVFENIENSQALVDQSNRILSSAEKGNTGGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 99 - protein, 751 aa, synthetic construct
  REGION 1..751: note = HMPV-F 115-BV (A185P) -foldon-I53-50A embodiment
  VARIANT 1..18: note = optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 100 - protein, 712 aa, synthetic construct
  REGION 1..712: note = HMPV-F 115-BV (A185P)-I53-50A - F10 embodiment
  VARIANT 1..18: note = optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 101 - protein, 490 aa, synthetic construct
  REGION 1..490: note = HMPV F >AAK62968.2 fusion protein [Human metapneumovirus]
  VARIANT 1..18: note = optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTG

(19) United States
(12) Patent Application Publication
(10) Pub. No.: US 2024/0150411 A1
(43) Pub. Date: May 9, 2024

(54) Self-assembling protein nanostructures displaying paramyxovirus and/or pneumovirus F proteins and their use

(71) Applicants: University of Washington, Seattle, WA (US); INSTITUTE FOR RESEARCH IN BIOMEDICINE, Bellinzona (CH)

(72) Inventors: Neil P. KING, Seattle, WA (US); David BAKER, Seattle, WA (US); Brooke FIALA, Seattle, WA (US); Lance Joseph STEWART, Seattle, WA (US); Laurent PEREZ, Bellinzona (CH); Antonio LANZAVECCHIA, Bellinzona (CH); Jessica MARCANDALLI, Bellinzona (CH)

(21) Appl. No.: 18/337,881

(22) Filed: Jun. 20, 2023

Related U.S. Application Data
(63) Continuation of application No. 17/523,174, filed on Nov. 10, 2021, now Pat. No. 11,732,011.
Application No. 17/523,174 is a division of application No. 16/500,331, filed on Oct. 2, 2019, now Pat. No. 11,192,926.
(60) Provisional application No. 62/481,331, filed on Apr. 4, 2017.

(51) Int. Cl. C07K 14/005; A61K 39/12; A61P 31/14; B82Y 5/00; C12N 7/00

(52) U.S. Cl. CPC C07K 14/005; A61K 39/12; A61P 31/14; B82Y 5/00; C12N 7/00; A61K 2039/575

(57) ABSTRACT

Disclosed herein are nanostructures and their use, where the nanostructures include
(a) a plurality of first assemblies, each first assembly comprising a plurality of identical first polypeptides;
(b) a plurality of second assemblies, each second assembly comprising a plurality of identical second polypeptides, wherein the second polypeptide differs from the first polypeptide;
wherein the plurality of first assemblies non-covalently interact with the plurality of second assemblies to form a nanostructure; and
wherein the nanostructure displays multiple copies of one or more paramyxovirus and/or pneumovirus F proteins or antigenic fragments thereof, on an exterior of the nanostructure.

### CROSS REFERENCE

This application is a continuation of U.S. patent application Ser. No. 17/523,174, filed Nov. 10, 2021, which is a divisional of U.S. patent application Ser. No. 16/500,331, filed Oct. 2, 2019 (now U.S. Pat. No. 11,192,926), which is a U.S. national phase of International Application No. PCT/US2018/025880, filed on Apr. 3, 2018, which claims priority to U.S. Provisional Application No. 62/481,331, filed Apr. 4, 2017, all of which are incorporated by reference herein in their entirety

### REFERENCE TO SEQUENCE LISTING SUBMITTED ELECTRONICALLY

The instant application contains a Sequence Listing which has been submitted electronically and is hereby incorporated by reference in its entirety. The Sequence Listing is contained in the file created on Jun. 15, 2023, having the file name “17-341-WO-US-DIV-CON_SequenceListing.xml” and is 225,280 bytes in size.

### BACKGROUND

Molecular self- and co-assembly of proteins into highly ordered, symmetric supramolecular complexes is an elegant and powerful means of patterning matter at the atomic scale. Recent years have seen advances in the development of self-assembling biomaterials, particularly those composed of nucleic acids. DNA has been used to create, for example, nanoscale shapes and patterns, molecular containers, and three-dimensional macroscopic crystals. Methods for designing self-assembling proteins have progressed more slowly, yet the functional and physical properties of proteins make them attractive as building blocks for the development of advanced functional materials.

### SUMMARY OF THE INVENTION

In one aspect, nanostructures are provided comprising:
(a) a plurality of first assemblies, each first assembly comprising a plurality of identical first polypeptides;
(b) a plurality of second assemblies, each second assembly comprising a plurality of identical second polypeptides, wherein the second polypeptide differs from the first polypeptide;
wherein the plurality of first assemblies non-covalently interact with the plurality of second assemblies to form a nanostructure; and
wherein the nanostructure displays multiple copies of one or more paramyxovirus and/or pneumovirus F proteins, or antigenic fragments thereof, on an exterior of the nanostructure.

In one embodiment, (a) the first polypeptides comprise a polypeptide having at least 70%, 75%, 80%, 85%, 90%, 91%, 92%, 93%, 94%, 95%, 96%, 97%, 98%, 99%, or 100% identity along its full length to the amino acid sequence of a polypeptide selected from the group consisting of SEQ ID NOS:1-51; and
(b) the second polypeptides comprise a polypeptide having at least 70%, 75%, 80%, 85%, 90%, 91%, 92%, 93%, 94%, 95%, 96%, 97%, 98%, 99%, or 100% identity along its full length to the amino acid sequence of a polypeptide selected from the group consisting of SEQ ID NOS:1-51.

In another embodiment, the one or more paramyxovirus and/or pneumovirus F proteins, or antigenic fragments thereof, comprise a polypeptide having at least 75%, 80%, 85%, 90%, 91%, 92%, 93%, 94%, 95%, 96%, 97%, 98%, 99%, or 100% identity along its full length to the amino acid sequence of a polypeptide selected from the group consisting of SEQ ID NOS: 53, 61-68, and 101.

In various embodiments:
(a) the first polypeptide comprises a polypeptide having at least 75%, 80%, 85%, 90%, 91%, 92%, 93%, 94%, 95%, 96%, 97%, 98%, 99%, or 100% identity along its full length to the amino acid sequence of T33-31A (SEQ ID NO:51) and the second polypeptide comprises a polypeptide having at least 75%, 80%, 85%, 90%, 91%, 92%, 93%, 94%, 95%, 96%, 97%, 98%, 99%, or 100% identity along its full length to the amino acid sequence of T33-09B/T33-31B (SEQ ID NO:44);
(b) the first polypeptide comprises a polypeptide having at least 75%, 80%, 85%, 90%, 91%, 92%, 93%, 94%, 95%, 96%, 97%, 98%, 99%, or 100% identity along its full length to the amino acid sequence of T33-15B (SEQ ID NO:46) and the second polypeptide comprises a polypeptide having at least 75%, 80%, 85%, 90%, 91%, 92%, 93%, 94%, 95%, 96%, 97%, 98%, 99%, or 100% identity along its full length to the amino acid sequence of T33-15A (SEQ ID NO:45);
(c) the first polypeptide has at least 75%, 80%, 85%, 90%, 91%, 92%, 93%, 94%, 95%, 96%, 97%, 98%, 99%, or 100% identity along its full length to the amino acid sequence of a polypeptide selected from the group consisting of I53-50A (SEQ ID NO:7), I53-50A.1 (SEQ ID NO:29), I53-50A.1NegT2 (SEQ ID NO:30), and I53-50A.1PosT1 (SEQ ID NO:31), and the second polypeptide has at least 75%, 80%, 85%, 90%, 91%, 92%, 93%, 94%, 95%, 96%, 97%, 98%, 99%, or 100% identity along its full length to the amino acid sequence of a polypeptide selected from the group consisting of I53-50B (SEQ ID NO:8), I53-50B.1 (SEQ ID NO:32), 153-50B.1NegT2 (SEQ ID NO:33), and I53-50B.4PosT1 (SEQ ID NO:34); or
(d) the first polypeptide comprises a polypeptide having at least 75%, 80%, 85%, 90%, 91%, 92%, 93%, 94%, 95%, 96%, 97%, 98%, 99%, or 100% identity along its full length to the amino acid sequence of I32-28A (SEQ ID NO:21) and the second polypeptide comprises a polypeptide having at least 75%, 80%, 85%, 90%, 91%, 92%, 93%, 94%, 95%, 96%, 97%, 98%, 99%, or 100% identity along its full length to the amino acid sequence of I32-28B (SEQ ID NO:22).

In one embodiment, the one or more paramyxovirus and/or pneumovirus F proteins, or antigenic fragments thereof, are expressed as a fusion protein with the first polypeptides. In one such embodiment, the plurality of first assemblies each comprise identical first polypeptides; in another such embodiment, the plurality of first assemblies in total comprise two or more paramyxovirus and/or pneumovirus F proteins, or antigenic fragments thereof. In another embodiment, only a subset of the first polypeptides comprise a fusion protein with an F protein or antigenic fragment thereof. In a further embodiment, each first assembly comprises a homotrimer of the first polypeptide.

In another embodiment, the fusion protein comprises an amino acid linker positioned between the first polypeptide and the paramyxovirus and/or pneumovirus F proteins, or antigenic fragment thereof. In one such embodiment, the fusion protein comprises an amino acid linker positioned between the first polypeptide and the paramyxovirus F proteins, or antigenic fragment thereof.

In one embodiment the amino acid linker sequence comprises one or more trimerization domain; in another embodiment the amino acid linker sequence comprises a Gly-Ser linker.

In various embodiments, the first polypeptides comprise or consist of first polypeptides having at least 75%, 80%, 85%, 90%, 91%, 92%, 93%, 94%, 95%, 96%, 97%, 98%, 99%, or 100% identity along their full length to the amino acid sequence of a polypeptide selected from the group consisting of DS-Cav1-foldon-T33-31A (SEQ ID NO:69), DS-Cav1-T33-31A (SEQ ID NO:70), DS-Cav1-foldon-T33-15B (SEQ ID NO:71), DS-Cav1-T33-15B (SEQ ID NO:72), DS-Cav1-foldon-I53-50A (SEQ ID NO:73), DS-Cav1-I53-50A (SEQ ID NO:74), and DS-Cav1-I32-28A (SEQ ID NO:75). In other embodiments,
(a) when each first polypeptide has at least 75%, 80%, 85%, 90%, 91%, 92%, 93%, 94%, 95%, 96%, 97%, 98%, 99%, or 100% identity along its full length to the amino acid sequence of DS-Cav1-foldon-T33-31A (SEQ ID NO:69) or DS-Cav1-T33-31A (SEQ ID NO:70), each second polypeptide has at least 75%, 80%, 85%, 90%, 91%, 92%, 93%, 94%, 95%, 96%, 97%, 98%, 99%, or 100% identity along its full length to the amino acid sequence of T33-31B (SEQ ID NO:44);
(b) when each first polypeptide has at least 75%, 80%, 85%, 90%, 91%, 92%, 93%, 94%, 95%, 96%, 97%, 98%, 99%, or 100% identity along its full length to the amino acid sequence of DS-Cav1-foldon-T33-15B (SEQ ID NO:71) or DS-Cav1-T33-15B (SEQ ID NO:72), each second polypeptide has at least 75%, 80%, 85%, 90%, 91%, 92%, 93%, 94%, 95%, 96%, 97%, 98%, 99%, or 100% identity along its full length to the amino acid sequence of T33-15A (SEQ ID NO:45);
(c) when each first polypeptide has at least 75%, 80%, 85%, 90%, 91%, 92%, 93%, 94%, 95%, 96%, 97%, 98%, 99%, or 100% identity along its full length to the amino acid sequence of DS-Cav1-foldon-I53-50A (SEQ ID NO:73) or DS-Cav1-I53-50A (SEQ ID NO:74), each second polypeptide has at least 75%, 80%, 85%, 90%, 91%, 92%, 93%, 94%, 95%, 96%, 97%, 98%, 99%, or 100% identity along its full length to the amino acid sequence of a polypeptide selected from the group consisting of I53-50B (SEQ ID NO:8), I53-50B.1 (SEQ ID NO:32), I53-50B.1NegT2 (SEQ ID NO:33), or I53-50B.4PosT1 (SEQ ID NO:34); or
(d) when each first polypeptide has at least 75%, 80%, 85%, 90%, 91%, 92%, 93%, 94%, 95%, 96%, 97%, 98%, 99%, or 100% identity along its full length to the amino acid sequence of DS-Cav1-I32-28A (SEQ ID NO:75), each second polypeptide has at least 75%, 80%, 85%, 90%, 91%, 92%, 93%, 94%, 95%, 96%, 97%, 98%, 99%, or 100% identity along its full length to the amino acid sequence of I32-28B.

In one embodiment, the one or more paramyxovirus and/or pneumovirus F proteins, or antigenic fragments thereof comprises a polypeptide having at least 75%, 80%, 85%, 90%, 91%, 92%, 93%, 94%, 95%, 96%, 97%, 98%, 99%, or 100% identity along its full length to the amino acid sequence of DS-Cav1(SEQ ID NO:53). In one such embodiment, each first polypeptide comprises a fusion polypeptide of a polypeptide having at least 75%, 80%, 85%, 90%, 91%, 92%, 93%, 94%, 95%, 96%, 97%, 98%, 99%, or 100% identity along its full length to the amino acid sequence of DS-Cav1 linked via an amino acid linker to a polypeptide having at least 75%, 80%, 85%, 90%, 91%, 92%, 93%, 94%, 95%, 96%, 97%, 98%, 99%, or 100% identity along its full length to the amino acid sequence of SEQ ID NO:7 (I53-50A). In another embodiment, the amino acid linker comprises a Gly-Ser linker. In a further embodiment, each fusion protein comprises a polypeptide having at least 75%, 80%, 85%, 90%, 91%, 92%, 93%, 94%, 95%, 96%, 97%, 98%, 99%, or 100% identity along its full length to the amino acid sequence selected from the group consisting of SEQ ID NOS:69-100. In a specific embodiment, each fusion protein comprises a polypeptide having at least 75%, 80%, 85%, 90%, 91%, 92%, 93%, 94%, 95%, 96%, 97%, 98%, 99%, or 100% identity along its full length to the amino acid sequence of SEQ ID NO:76 (F10). In other embodiments, each second polypeptide comprises a polypeptide having at least 75%, 80%, 85%, 90%, 91%, 92%, 93%, 94%, 95%, 96%, 97%, 98%, 99%, or 100% identity along its full length to the amino acid sequence selected from the group consisting of I53-50B (SEQ ID NO:8), I53-50B.1 (SEQ ID NO:32), I53-50B.1NegT2 (SEQ ID NO:33), or I53-15 50B.4PosT1 (SEQ ID NO:34). In a specific embodiment, each second polypeptide comprises a polypeptide having at least 75%, 80%, 85%, 90%, 91%, 92%, 93%, 94%, 95%, 96%, 97%, 98%, 99%, or 100% identity along its full length to the amino acid sequence of I53-50B.4PosT1 (SEQ ID NO:34).

In other aspects, recombinant expression nucleic acids expressing the first polypeptide fusions, recombinant expression vectors comprising the recombinant nucleic acids linked to a promoter, and recombinant host cells comprising the recombinant expression vectors are provided.

Also provided are immunogenic compositions comprising the nanostructure of any embodiment or combination of embodiments disclosed herein, and a pharmaceutically acceptable carrier. In one embodiment, the immunogenic compositions may further comprise an adjuvant.

In other aspects, methods for generating an immune response to RSV F protein in a subject, or for treating or limiting a RSV infection in a subject are provided, comprising administering to the subject in need thereof an effective amount of the nanostructure or immunogenic composition of embodiment or combination of embodiments disclosed herein to generate the immune response or to treat or prevent RSV infection in the subject.

Also provided are processes assembling the nanostructures of any embodiment or combination of embodiments disclosed herein, comprising mixing two or more nanostructures components in aqueous conditions to drive spontaneous assembly of the desired nanostructures.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 shows a schematic diagram of the production of antigen-bearing nanostructures by in vitro assembly. The two components or building blocks of a given nanostructure can be expressed and purified individually, which allows assembly of the nanostructure to be initiated by mixing the purified components in vitro, a process referred to as in vitro assembly. In some embodiments, the two components of the nanostructure may be expressed in different expression hosts (e.g., human HEK293F cells or bacterial E. coli cells). The figure schematically depicts assembly of a 120-subunit nanostructure bearing 20 trimeric antigens (60 antigen subunits) via in vitro assembly of an antigen-nanostructure trimer fusion protein produced in HEK293F cells and a nanostructure pentamer protein produced in E. coli.

FIG. 2 shows graphs illustrating detection of secreted DS-Cav1, DS-Cav1-foldon-T33-31A, and DS-Cav1-T33-31A fusion proteins in tissue culture supernatants. ELISA assays were performed on tissue culture supernatants from cells expressing DS-Cav1 (top), DS-Cav-1-foldon-T33-31A/T33-31B (bottom left), and DS-Cav-1-T33-31A/T33-31B (bottom right). Four different monoclonal antibodies that bind RSV F were used to evaluate the presence of DS-Cav1 or DS-Cav1 fusion proteins in the supernatants. The results confirm the secretion of proteins comprising well-folded RSV F antigen.

FIG. 3 shows size-exclusion chromatography of DS-Cav1-I53-50A. Protein purified from tissue culture supernatants by immobilized metal affinity chromatography was applied to a Superose™ 6 10/300 GL size exclusion column. The protein eluted as a single, monodisperse species.

FIG. 4 shows size exclusion chromatography of in vitro-assembled DS-Cav1-I53-50 nanostructures. Purified DS-Cav1-I53-50A and I53-50B.4PT1 proteins were mixed at an approximately 1:1 molar ratio, incubated overnight at 4° C., and then applied to a Sephacryl S-500 16/60 HR size exclusion column. The assembled nanostructure eluted as a single, monodisperse peak around 65 mL, while excess DS-Cav1-I53-50A trimeric component eluted around 90 mL.

FIG. 5 shows a negative stain electron micrograph and two-dimensional class averages of in vitro-assembled DS-Cav1-I53-50 nanostructures. In vitro-assembled DS-Cav1-I53-50 nanostructures, purified by size exclusion chromatography, were imaged by negative stain electron microscopy (top). Averaging many nanostructures yielded two-dimensional class averages (bottom) that indicate that the I53-50 portion of the nanostructures is highly ordered and consistent, while the precise three-dimensional position of the displayed antigen varies slightly due to the flexible nature of the linker between the DS-Cav1 and I53-50A domains of the DS-Cav1-I53-50A fusion protein.

FIG. 6A-6C show a series of graphs depicting the antigenicity of DS-Cav1-I53-50 nanostructures. Analysis of purified DS-Cav1-I53-50 nanostructures by ELISA (FIG. 6A) using four RSV F-specific monoclonal antibodies, including the prefusion-specific antibodies MPE8, D25, and RSD5, indicated that the DS-Cav1 antigen is correctly folded and maintained in the prefusion state when multivalently displayed on DS-Cav1-I53-50 nanostructures. This finding was confirmed by surface plasmon resonance measurements using multiple RSV F-specific antibodies (FIG. 6B-6C), which, when compared to trimeric DS-Cav1, further suggested that multivalent display of DS-Cav1 results in an avidity effect that reduces the dissociation rate of the antibodies.

FIG. 7 is a graph depicting DS-Cav1-specific serum antibody titers from mice immunized with DS-Cav1-I53-50 nanostructures. Groups of mice were immunized with I53-50 nanostructures lacking additional antigen, trimeric DS-Cav1, or I53-50 nanostructures bearing DS-Cav1 antigen at 33%, 66%, or 100% valency. DS-Cav1-specific serum antibody titers were measured by ELISA on plates coated with DS-Cav1. Serum antibody titers for each mouse are plotted as circles, with the geometric mean within each group plotted as a horizontal line.

FIG. 8 is a graph depicting serum neutralization activity elicited by immunization with DS-Cav1-I53-50 nanostructures. Groups of mice were immunized with I53-50 nanostructures lacking additional antigen, trimeric DS-Cav1, or I53-50 nanostructures bearing DS-Cav1 antigen at 33%, 66%, or 100% valency. Neutralization titers for each mouse are plotted as circles, with the geometric mean within each group plotted as a horizontal line.

FIG. 9A-9B are graphs depicting immunogenicity in a primate immune system elicited by immunization with DS-Cav1-foldon I53-50 nanostructures. Rhesus macaques were injected at weeks 0 and 4 with either free DS-Cav1 trimer or DS-Cav1-foldon-I53-50 nanostructures displaying DS-Cav1 at 100% valency. In both cases, the dose of DS-Cav1 antigen was 50 μg, and the immunogens were formulated with the MF59-like, squalene-based oil-in-water emulsion adjuvant SWE. Sera obtained from the animals at weeks 6 and 16 were evaluated for anti-DS-Cav1 antibody titers (FIG. 9A) and RSV-neutralizing antibody titers (FIG. 9B).

FIG. 10 is a graph depicting the physical stability of DS-Cav1 when fused to I53-50A and/or when further assembled into the icosahedral nanostructure. Samples of trimeric DS-Cav1, trimeric DS-Cav1-foldon-I53-50A, and DS-Cav1-foldon-I53-50 nanostructures containing equivalent concentrations of DS-Cav1 were split into four aliquots and incubated at 20, 50, 70 or 80° C. for 1 hour. After cooling to room temperature, D25 binding was assayed by surface plasmon resonance (SPR).

FIG. 11A-11J are graphs depicting physical stability of the nanostructures. Chemical denaturation in guanidine hydrochloride (GdnHCl), monitored by intrinsic tryptophan fluorescence, was used as a second, antibody-independent technique to evaluate physical stability of trimeric DS-Cav1 (FIG. 11A-11B), DS-Cav1-foldon-I53-50A (FIG. 11C-11D), DS-Cav1-foldon-I53-50 (FIG. 11E-11F), I53-50 (FIG. 11G-11H), and I53-50A (FIG. 11I-11J). The data indicate superior physical stability of the DS-Cav1 antigen when genetically fused to the I53-50A nanostructure component.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, “about” means +/−5% of the recited parameter.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the disclosure provides nanostructures, comprising:
(a) a plurality of first assemblies, each first assembly comprising a plurality of identical first polypeptides;
(b) a plurality of second assemblies, each second assembly comprising a plurality of identical second polypeptides, wherein the second polypeptide differs from the first polypeptide;
wherein the plurality of first assemblies non-covalently interact with the plurality of second assemblies to form a nanostructure; and
wherein the nanostructure displays multiple copies of one or more paramyxovirus and/or pneumovirus F proteins, or antigenic fragments thereof, on an exterior of the nanostructure.

Self-assembling polypeptide nanostructures are disclosed herein that multivalently display paramyxovirus and/or pneumovirus F proteins on the nanostructure exteriors. Multiple copies of pairs of first and second polypeptides are able to self-assemble to form nanostructures, such as icosahedral nanostructures. The nanostructures include symmetrically repeated, non-natural, non-covalent polypeptide-polypeptide interfaces that orient a first assembly and a second assembly into a nanostructure, such as one with an icosahedral symmetry.

The nanostructures of the invention are synthetic, in that they are not naturally occurring. The first polypeptides and the second polypeptides are non-naturally occurring proteins that can be produced by any suitable means, including recombinant production or chemical synthesis. Each member of the plurality of first polypeptides is identical to each other (though when the first polypeptide is present as a fusion polypeptide with one or more paramyxovirus and/or pneumovirus F proteins, or antigenic fragments thereof, the F protein or antigenic fragment thereof may differ from one first polypeptide to another), and each member of the plurality of second polypeptides is identical to each other. The first proteins and the second proteins are different. There are no specific primary amino acid sequence requirements for the first and second polypeptides. US published patent application 20160122392 and published PCT application WO2014/124301 describe methods for designing synthetic nanostructures, where the nanostructures are not dependent on specific primary amino acid sequences of the first and second polypeptides.

A plurality (2, 3, 4, 5, 6, or more) of first polypeptides self-assemble to form a first assembly, and a plurality (2, 3, 4, 5, 6, or more) of second polypeptides self-assemble to form a second assembly. A plurality of these first and second assemblies then self-assemble non-covalently via the designed interfaces to produce the nanostructures.

The number of first polypeptides in the first assemblies may be the same or different than the number of second polypeptides in the second assemblies. In one exemplary embodiment, the first assembly comprises trimers of the first polypeptides, and the second assembly comprises dimers of the second polypeptides. In a further exemplary embodiment, the first assembly comprises trimers of the first polypeptides, and the second assembly comprises trimers of the second polypeptides. In a further exemplary embodiment, the first assembly comprises trimers of the first polypeptides, and the second assembly comprises pentamers of the second polypeptides.

The first and second polypeptides may be of any suitable length for a given purpose of the resulting nanostructure. In one embodiment, the first polypeptides and the second polypeptides are typically between 30-250 amino acids in length; the length of the first polypeptides and the second polypeptides may be the same or different. In various further embodiments, the first polypeptides and the second polypeptides are between 30-225, 30-200, 30-175, 50-250, 50-225, 50-200, 50-175, 75-250, 75-225, 75-200, 75-175, 100-250, 100-225, 100-200, 100-175, 125-250, 125-225, 125-200, 125-175, 150-250, 150-225, 150-200, and 150-175 amino acids in length.

The isolated polypeptides of SEQ ID NOS:1-51 were designed for their ability to self-assemble in pairs to form nanostructures, such as icosahedral nanostructures. The design involved design of suitable interface residues for each member of the polypeptide pair that can be assembled to form the nanostructure. The nanostructures so formed include symmetrically repeated, non-natural, non-covalent polypeptide-polypeptide interfaces that orient a first assembly and a second assembly into a nanostructure, such as one with an icosahedral symmetry. Thus, in one embodiment the first and second polypeptides are selected from the group SEQ ID NOS:1-51. In each case, the N-terminal methionine residue is optional.

TABLE 1

| | | Identified interface |
|---|---|---|
| Name | Amino Acid Sequence | residues |
| I53-34A | (M)EGMDPLAVLAESRLLPLLTVRGGEDLAGLATVLELMGV | I53-34A: |
| SEQ ID | GALEITLRTEKGLEALKALRKSGLLLGAGTVRSPKEAEAAL | 28, 32, 36, 37, 186, 188, 191, |
| NO: 1 | EAGAAFLVSPGLLEEVAALAQARGVPYLPGVLTPTEVERAL | 192, 195 |
| | ALGLSALKFFPAEPFQGVRVLRAYAEVFPEVRFLPTGGIKE | |
| | EHLPHYAALPNLLAVGGSWLLQGDLAAVMKKVKAAKALLSP | |
| | QAPG | |
| I53-34B | (M)TKKVGIVDTTFARVDMAEAAIRTLKALSPNIKIIRKTV | I53-34B: |
| SEQ ID | PGIKDLPVACKKLLEEEGCDIVMALGMPGKAEKDKVCAHEA | 19, 20, 23, 24, 27, 109, 113, 116, |
| NO: 2 | SLGLMLAQLMTNKHIIEVFVHEDEAKDDDELDILALVRAIE | 117, 120, 124, 148 |
| | HAANVYYLLFKPEYLTRMAGKGLRQGREDAGPARE | |
| I53-40A | (M)TKKVGIVDTTFARVDMASAAILTLKMESPNIKIIRKTV | I53-40A: |
| SEQ ID | PGIKDLPVACKKLLEEEGCDIVMALGMPGKAEKDKVCAHEA | 20, 23, 24, 27, 28, 109, 112, 113, |
| NO: 3 | SLGLMLAQLMTNKHIIEVFVHEDEAKDDAELKILAARRAIE | 116, 120, 124 |
| | HALNVYYLLFKPEYLTRMAGKGLRQGFEDAGPARE | |
| I53-40B | (M)STINNQLKALKVIPVIAIDNAEDIIPLGKVLAENGLPA | I53-40B: |
| SEQ ID | AEITFRSSAAVKAIMLLRSAQPEMLIGAGTILNGVQALAAK | 47, 51, 54, 58, 74, 102 |
| NO: 4 | EAGATFVVSPGFNPNTVRACQIIGIDIVPGVNNPSTVEAAL | |
| | EMGLITLKFFPAEASGGISMVKSLVGPYGDIRLMPTGGITP | |
| | SNIDNYLAIPQVLACGGTWMVDKKLVTNGEWDEIARLTREI | |
| | VEQVNP | |
| I53-47A | (M)PIFTLNTNIKATDVPSDFLSLTSRLVGLILSKPGSYVA | I53-47A: |
| SEQ ID | VHINTDQQLSFGGSTNPAAFGTLMSIGGIEPSKNRDHSAVL | 22, 25, 29, 72, 79, 86, 87 |
| NO: 5 | FDHLNAMLGIPKNRMYIHFVNLNGDDVGWNGTTF | |
| I53-47B | (M)NQHSHKDYETVRIAVVRARWHADIVDACVEAFEIAMAA | I53-47B: |
| SEQ ID | IGGDRFAVDVFDVPGAYEIPLHARTLAETGRYGAVLGTAFV | 28, 31, 35, 36, 39, 131, 132, 135, |
| NO: 6 | VNGGIYRHEFVASAVIDGMMNVQLSTGVPVLSAVLTPHRYR | 139, 146 |
| | DSAEHHRFFAAHFAVKGVEAARACIEILAAREKIAA | |
| I53-50A | (M)KMEELFKKHKIVAVLRANSVEEAIEKAVAVFAGGVHLI | I53-50A: 25, 29, 33, 54, 57 |
| SEQ ID | EITFTVPDADTVIKALSVLKEKGAIIGAGTVTSVEQCRKAV | |
| NO: 7 | ESGAEFIVSPHLDEEISQFCKEKGVFYMPGVMTPTELVKAM | |
| | KLGHTILKLFPGEVVGPQFVKAMKGPFPNVKFVPTGGVNLD | |
| | NVCEWFKAGVLAVGVGSALVKGTPDEVREKAKAFVEKIRGC | |
| | TE | |
| I53-50B | (M)NQHSHKDYETVRIAVVRARWHAEIVDACVSAFEAAMAD | I53-50B: |
| SEQ ID | IGGDRFAVDVFDVPGAYEIPLHARTLAETGRYGAVLGTAFV | 24, 28, 36, 124, 125, 127, 128, |
| NO: 8 | VNGGIYRHEFVASAVIDGMMNVQLSTGVPVLSAVLTPHRYR | 129, 131, 132, 133, 135, 139 |
| | DSDAHILLFLALFAVKGMEAARACVEILAAREKIAA | |
| I53-51A | (M)FTKSGDDGNTNVINKRVGKDSPLVNFLGDLDELNSFIG | I53-51A: |
| SEQ ID | FAISKIPWEDMKKDLERVQVELFEIGEDLSTQSSKKKIDES | 80, 83, 86, 87, 88, 90, 91, 94, 166, |
| NO: 9 | YVLWLLAATAIYRIESGPVKLFVIPGGSEEASVLHVTRSVA | 172, 176 |
| | RRVERNAVKYTKELPEINRMIIVYLNRLSSLLFAMALVANK | |
| | RRNQSEKIYEIGKSW | |
| I53-51B | (M)NQHSHKDYETVRIAVVRARWHADIVDQCVRAFEEAMAD | I53-51B: |
| SEQ ID | AGGDRFAVDVFDVPGAYEIPLHARTLAETGRYGAVLGTAFV | 31, 35, 36, 40, 122, 124, 128, |
| NO: 10 | VNGGIYRHEFVASAVIDGMMNVQLSTGVPVLSAVLTPHRYR | 131, 135, 139, 143, 146, 147 |
| | SSREHHEFFREHFMVKGVEAAAACITILAAREKIAA | |
| I52-03A | (M)GHTKGPTPQQHDGSALRIGIVHARWNKTIIMPLLIGTI | I52-03A: |
| SEQ ID | AKLLECGVKASNIVVQSVPGSWELPIAVQRLYSASQLQTPS | 28, 32, 36, 39, 44, 49 |
| NO: 11 | SGPSLSAGDLLGSSTTDLTALPTTTASSTGPFDALIAIGVL | |
| | IKGETMHFEYIADSVSHGLMRVQLDTGVPVIFGVLTVLTDD | |
| | QAKARAGVIEGSHNHGEDWGLAAVEMGVRRRDWAAGKTE | |
| I52-03B | (M)YEVDHADVYDLFYLGRGKDYAAEASDIADLVRSRTPEA | I52-03B: |
| SEQ ID | SSLLDVACGTGTHLEHFTKEFGDTAGLELSEDMLTHARKRL | 94, 115, 116, 206, 213 |
| NO: 12 | PDATLHQGDMRDFQLGRKFSAVVSMFSSVGYLKTVAELGAA | |
| | VASFAEHLEPGGVVVVEPWWFPETFADGWVSADVVRRDGRT | |
| | VARVSHSVREGNATRMEVHFTVADPGKGVRHFSDVHLITLF | |
| | HQREYEAAFMAAGLRVEYLEGGPSGRGLFVGVPA | |
| I52-32A | (M)GMKEKFVLIITHGDFGKGLLSGAEVIIGKQENVHTVGL | I52-32A: |
| SEQ ID | NLGDNIEKVAKEVMRIIIAKLAEDKEIIIVVDLFGGSPFNI | 47, 49, 53, 54, 57, 58, 61, 83, 87, |
| NO: 13 | ALEMMKTFDVKVITGINMPMLVELLTSINVYDTTELLENIS | 88 |
| | KIGKDGIKVIEKSSLKM | |
| I52-32B | (M)KYDGSKLRIGILHARWNLEIIAALVAGAIKRLQEFGVK | I52-32B: 19, 20, 23, 30, 40 |
| SEQ ID | AENIIIETVPGSFELPYGSKLFVEKQKRLGKPLDAIIPIGV | |
| NO: 14 | LIKGSTMHFEYICDSTTHQLMKLNFELGIPVIFGVLTCLTD | |
| | EQAEARAGLIEGKMHNHGEDWGAAAVEMATKFN | |
| I52-33A | (M)AVKGLGEVDQKYDGSKLRIGILHARWNRKIILALVAGA | I52-33A: 33, 41, 44, 50 |
| SEQ ID | VLRLLEFGVKAENIIIETVPGSFELPYGSKLFVEKQKRLGK | |
| NO: 15 | PLDAIIPIGVLIKGSTMHFEYICDSTTHQLMKLNFELGIPV | |
| | IFGVLTCLTDEQAEARAGLIEGKMHNHGEDWGAAAVEMATK | |
| | FN | |
| I52-33B | (M)GANWYLDNESSRLSFTSTKNADIAEVHRFLVLHGKVDP | I52-33B: |
| SEQ ID | KGLAEVEVETESISTGIPLRDMLLRVLVFQVSKFPVAQINA | 61, 63, 66, 67, 72, 147, 148, 154, |
| NO: 16 | QLDMRPINNLAPGAQLELRLPLTVSLRGKSHSYNAELLATR | 155 |
| | LDERRFQVVTLEPLVIHAQDFDMVRAFNALRLVAGLSAVSL | |
| | SVPVGAVLIFTAR | |
| I32-06A | (M)TDYIRDGSAIKALSFAIILAEADLRHIPQDLQRLAVRV | I32-06A: |
| SEQ ID | IHACGMVDVANDLAFSEGAGKAGRNALLAGAPILCDARMVA | 9, 12, 13, 14, 20, 30, 33, 34 |
| NO: 17 | EGITRSRLPADNRVIYTLSDPSVPELAKKIGNTRSAAALDL | |
| | WLPHIEGSIVAIGNAPTALFRLFELLDAGAPKPALIIGMPV | |
| | GFVGAAESKDELAANSRGVPYVIVRGRRGGSAMTAAAVNAL | |
| | ASERE | |
| I32-06B | (M)ITVFGLKSKLAPRREKLAEVIYSSLHLGLDIPKGKHAI | I32-06B: |
| SEQ ID | RFLCLEKEDFYYPFDRSDDYTVIEINLMAGRSEETKMLLIF | 24, 71, 73, 76, 77, 80, 81, 84, 85, |
| NO: 18 | LLFIALERKLGIRAHDVEITIKEQPAHCWGFRGRTGDSARD | 88, 114, 118 |
| | LDYDIYV | |
| I32-19A | (M)GSDLQKLQRFSTCDISDGLLNVYNIPIGGYFPNLTAIS | I32-19A: |
| SEQ ID | PPQNSSIVGTAYTVLFAPIDDPRPAVNYIDSVPPNSILVLA | 208, 213, 218, 222, 225, 226, |
| NO: 19 | LEPHLQSQFHPFIKITQAMYGGLMSTRAQYLKSNGTVVFGR | 229, 233 |
| | IRDVDEHRTLNHPVFAYGVGSCAPKAVVKAVGTNVQLKILT | |
| | SDGVTQTICPGDYIAGDNNGIVRIPVQETDISKLVTYIEKS | |
| | IEVDRLVSEAIKNGLPAKAAQTARRMVLKDYI | |
| I32-19B | (M)SGMRVYLGADHAGYELKQAIIAFLKMTGHEPIDCGALR | I32-19B: |
| SEQ ID | YDADDDYPAFCIAAATRTVADPGSLGIVLGGSGNGEQIAAN | 20, 23, 24, 27, 117, 118, 122, |
| NO: 20 | KVPGARCALAWSVQTAALAREHNNAQLIGIGGRMHTLEEAL | 125 |
| | RIVKAFVTTPWSKAQRHQRRIDILAEYERTHEAPPVPGAPA | |
| I32-28A | (M)GDDARIAAIGDVDELNSQIGVLLAEPLPDDVRAALSAI | I32-28A: |
| SEQ ID | QHDLFDLGGELCIPGHAAITEDHLLRLALWLVHYNGQLPPL | 60, 61, 64, 67, 68, 71, 110, 120, |
| NO: 21 | EEFILPGGARGAALAHVCRTVCRRAERSIKALGAsEPLNIA | 123, 124, 128 |
| | PAAYVNLLSDLLFVLARVLNRAAGGADVLWDRTRAH | |
| I32-28B | (M)ILSAEQSFTLRHPHGQAAALAFVREPAAALAGVQRLRG | I32-28B: |
| SEQ ID | LDSDGEQVWGELLVRVPLLGEVDLPFRSEIVRTPQGAELRP | 35, 36, 54, 122, 129, 137, 140, |
| NO: 22 | LTLTGERAWVAVSGQATAAEGGEMAFAFQFQAHLATPEAEG | 141, 144, 148 |
| | EGGAAFEVMVQAAAGVTLLLVAMALPQGLAAGLPPA | |
| I53- | (M)TKKVGIVDTTFARVDMASAAILTLKMESPNIKIIRKTV | I53-40A: |
| 40A.1 | PGIKDLPVACKKLLEEEGCDIVMALGMPGKKEKDKVCAHEA | 20, 23, 24, 27, 28, 109, 112, 113, |
| SEQ ID | SLGLMLAQLMTNKHIIEVFVHEDEAKDDAELKILAARRAIE | 116, 120, 124 |
| NO: 23 | HALNVYYLLFKPEYLTRMAGKGLRQGFEDAGPARE | |
| I53- | (M)DDINNQLKRLKVIPVIAIDNAEDIIPLGKVLAENGLPA | I53-40B: |
| 40B.1 | AEITFRSSAAVKAIMLLRSAQPEMLIGAGTILNGVQALAAK | 47, 51, 54, 58, 74, 102 |
| SEQ ID | EAGADFVVSPGFNPNTVRACQIIGIDIVPGVNNPSTVEQAL | |
| NO: 24 | EMGLTTLKFFPAEASGGISMVKSLVGPYGDIRLMPTGGITP | |
| | DNIDNYLAIPQVLACGGTWMVDKKLVRNGEWDEIARLTREI | |
| | VEQVNP | |
| I53- | (M)PIFTLNTNIKADDVPSDFLSLTSRLVGLILSKPGSYVA | I53-47A: |
| 47A.1 | VHINTDQQLSFGGSTNPAAFGTLMSIGGIEPDKNRDHSAVL | 22, 25, 29, 72, 79, 86, 87 |
| SEQ ID | FDHLNAMLGIPKNRMYIHFVNLNGDDVGWNGTTF | |
| NO: 25 | | |
| I53- | (M)PIFTLNTNIKADDVPSDFLSLTSRLVGLILSEPGSYVA | I53-47A: |
| 47A.1NegT2 | VHINTDQQLSFGGSTNPAAFGTLMSIGGIEPDKNEDHSAVL | 22, 25, 29, 72, 79, 86, 87 |
| SEQ ID | FDHLNAMLGIPKNRMYIHFVDLDGDDVGWNGTTF | |
| NO: 26 | | |
| I53- | (M)NQHSHKDHETVRIAVVRARWHADIVDACVEAFEIAMAA | I53-47B: |
| 47B.1 | IGGDRFAVDVFDVPGAYEIPLHARTLAETGRYGAVLGTAFV | 28, 31, 35, 36, 39, 131, 132, 135, |
| SEQ ID | VNGGIYRHEFVASAVIDGMMNVQLDTGVPVLSAVLTPHRYR | 139, 146 |
| NO: 27 | DSDEHHRFFAAHFAVKGVEAARACIEILNAREKIAA | |
| I53- | (M)NQHSHKDHETVRIAVVRARWHADIVDACVEAFEIAMAA | I53-47B: |
| 47B.1NegT2 | IGGDRFAVDVFDVPGAYEIPLHARTLAETGRyGAVLGTAFV | 28, 31, 35, 36, 39, 131, 132, 135, |
| SEQ ID | VDGGIYDHEFVASAVIDGMMNVQLDTGVPVLSAVLTPHEYE | 139, 146 |
| NO: 28 | DSDEDHEFFAAHFAVKGVEAARACIEILNAREKIAA | |
| I53- | (M)KMEELFKKHKIVAVLRANSVEEAIEKAVAVFAGGVHLI | I53-50A: 25, 29, 33, 54, 57 |
| 50A.1 | EITFTVPDADTVIKALSVLKEKGAIIGAGTVTSVEQCRKAV | |
| SEQ ID | ESGAEFIVSPHLDEEISQFCKEKGVFYMPGVMTPTELVKAM | |
| NO: 29 | KLGHDILKLFPGEVVGPQFVKAMKGPFPNVKFVPTGGVNLD | |
| | NVCEWFKAGVLAVGVGDALVKGDPDEVREKAKKFVEKIRGC | |
| | TE | |
| I53- | (M)KMEELFKKHKIVAVLRANSVEEAIEKAVAVFAGGVHLI | I53-50A: 25, 29, 33, 54, 57 |
| 50A.1NegT2 | EITFTVPDADTVIKALSVLKEKGAIIGAGTVTSVEQCRKAV | |
| SEQ ID | ESGAEFIVSPHLDEEISQFCKEKGVFYMPGVMTPTELVKAM | |
| NO: 30 | KLGHDILKLFPGEVVGPEFVEAMKGPFPNVKFVPTGGVDLD | |
| | DVCEWFDAGVLAVGVGDALVEGDPDEVREDAKEFVEEIRGC | |
| | TE | |
| I53- | (M)KMEELFKKHKIVAVLRANSVEEAIEKAVAVFAGGVHLI | I53-50A: 25, 29, 33, 54, 57 |
| 50A.1PosT1 | EITFTVPDADTVIKALSVLKEKGAIIGAGTVTSVEQCRKAV | |
| SEQ ID | ESGAEFIVSPHLDEEISQFCKEKGVFYMPGVMTPTELVKAM | |
| NO: 31 | KLGHDILKLFPGEVVGPQFVKAMKGPFPNVKFVPTGGVNLD | |
| | NVCKWFKAGVLAVGVGKALVKGKPDEVREKAKKFVKKIRGC | |
| | TE | |
| I53- | (M)NQHSHKDHETVRIAVVRARWHAEIVDACVSAFEAAMRD | I53-50B: |
| 50B.1 | IGGDRFAVDVFDVPGAYEIPLHARTLAETGRYGAVLGTAFV | 24, 28, 36, 124, 125, 127, 128, |
| SEQ ID | VNGGIYRHEFVASAVIDGMMNVQLDTGVPVLSAVLTPHRYR | 129, 131, 132, 133, 135, 139 |
| NO: 32 | DSDAHILLFLALFAVKGMEAARACVEILAAREKIAA | |
| I53- | (M)NQHSHKDHETVRIAVVRARWHAEIVDACVSAFEAAMRD | I53-50B: |
| 50B.1NegT2 | IGGDRFAVDVFDVPGAYEIPLHARTLAETGRYGAVLGTAFV | 24, 28, 36, 124, 125, 127, 128, |
| SEQ ID | VDGGIYDHEFVASAVIDGMMNVQLDTGVPVLSAVLTPHEYE | 129, 131, 132, 133, 135, 139 |
| NO: 33 | DSDADTLLFLALFAVKGMEAARACVEILAAREKIAA | |
| I53- | (M)NQHSHKDHETVRIAVVRARWHAEIVDACVSAFEAAMRD | I53-50B: |
| 50B.4PosT1 | IGGDRFAVDVFDVPGAYEIPLHARTLAETGRYGAVLGTAFV | 24, 28, 36, 124, 125, 127, 128, |
| SEQ ID | VNGGIYRHEFVASAVINGMMNVQLNTGVPVLSAVLTPHNYD | 129, 131, 132, 133, 135, 139 |
| NO: 34 | KSKAHTLLFLALFAVKGMEAARACVEILAAREKIAA | |

I53-40A genus
(SEQ ID NO: 35)
(M)TKKVGIVDTTFARVDMASAAILTLKMESPNIKIIRKTVPGIKDLPVACKKLLEEEGCDIVMA
LGMPGK(A/K)EKDKVCAHEASLGLMLAQLMTNKHIIEVFVHEDEAKDDAELKILAARRAIEHAL
NVYYLLFKPEYLTRMAGKGLRQGFEDAGPARE
I53-40B genus
(SEQ ID NO: 36)
(M)
(S/D)(T/D)INNQLK(A/R)LKVIPVIAIDNAEDIIPLGKVLAENGLPAAEITFRSSAAVKAIM
LLRSAQPEMLIGAGTILNGVQALAAKEAGA(T/D)FVVSPGFNPNTVRACQIIGIDIVPGVNNPS
TVE(A/Q)ALEMGLTTLKFFPAEASGGISMVKSLVGPYGDIRLMPTGGITP(S/D)NIDNYLAIP
QVLACGGTWMVDKKLV(T/R)NGEWDEIARLTREIVEQVNP
I53-47A genus
(SEQ ID NO: 37)
(M)PIFTLNTNIKA(T/D)DVPSDFLSLTSRLVGLILS(K/E)PGSYVAVHINTDQQLSFGGSTN
PAAFGTLMSIGGIEP(S/D)KN(R/E)DHSAVLFDHLNAMLGIPKNRMYIHFV(N/D)L(N/D)G
DDVGWNGTTF
I53-47B genus
(SEQ ID NO: 38)
(M)NQHSHKD(Y/H)ETVRIAVVRARWHADIVDACVEAFEIAMAAIGGDRFAVDVFDVPGAYEIP
LHARTLAETGRYGAVLGTAFVV(N/D)GGIY(R/D)HEFVASAVIDGMMNVQL(S/D)TGVPVLS
AVLTPH(R/E)Y(R/E)DS(A/D)E(H/D)H(R/E)FFAAHFAVKGVEAARACIEIL(A/N)ARE
KIAA
I53-50A genus
(SEQ ID NO: 39)
(M)KMEELFKKHKIVAVLRANSVEEAIEKAVAVFAGGVHLIEITFTVPDADTVIKALSVLKEKGA
IIGAGTVISVEQCRKAVESGAEFIVSPHLDEEISQFCKEKGVFYMPGVMTPTELVKAMKLGH(T/
D)ILKLFPGEVVGP(Q/E)FV(K/E)AMKGPFPNVKFVPTGGV(N/D)LD(N/D)VC(E/K)WF
(K/D)AGVLAVGVG(S/K/D)ALV(K/E)G(T/D/K)PDEVRE(K/D)AK(A/E/K)FV(E/K)
(K/E)IRGCTE
I53-50B genus
(SEQ ID NO: 40)
(M)NQHSHKD(Y/H)ETVRIAVVRARWHAEIVDACVSAFEAAM(A/R)DIGGDRFAVDVFDVPGA
YEIPLHARTLAETGRYGAVLGTAFVV(N/D)GGIY(R/D)HEFVASAVI(D/N)GMMNVQL(S/
D/N)TGVPVLSAVLTPH(R/E/N)Y(R/D/E)(D/K)S(D/K)A(H/D)TLLFLALFAVKGMEAA
RACVEILAAREKIAA
T32-28A
(SEQ ID NO: 41)
(M)GEVPIGDPKELNGMEIAAVYLQPIEMEPRGIDLAASLADIHLEADIHALKNNPNGFPEGFWM
PYLTIAYALANADTGAIKTGTLMPMVADDGPHYGANIAMEKDKKGGFGVGTYALTFLISNPEKQG
FGRHVDEETGVGKWFEPFVVTYFFKYTGTPK
T32-28B
(SEQ ID NO: 42)
(M)SQAIGILELTSIAKGMELGDAMLKSANVDLLVSKTISPGKFLLMLGGDIGAIQQAIETGTSQ
AGEMLVDSLVLANIHPSVLPAISGLNSVDKRQAVGIVETWSVAACISAADLAVKGSNVTLVRVHM
AFGIGGKCYMVVAGDVLDVAAAVATASLAAGAKGLLVYASIIPRPHEAMWRQMVEG
T33-09A
(SEQ ID NO: 43)
(M)EEVVLITVPSALVAVKIAHALVEERLAACVNIVPGLTSIYRWQGSVVSDHELLLLVKTTTHA
FPKLKERVKALHPYTVPEIVALPIAEGNREYLDWLRENTG
T33-09B
(SEQ ID NO: 44)
(M)VRGIRGAITVEEDTPAAILAATIELLLKMLEANGIQSYEELAAVIFTVTEDLTSAFPAEAAR
LIGMHRVPLLSAREVPVPGSLPRVIRVLALWNTDTPQDRVRHVYLNEAVRLRPDLESAQ
T33-15A
(SEQ ID NO: 45)
(M)SKAKIGIVTVSDRASAGITADISGKAIILALNLYLTSEWEPIYQVIPDEQDVIETTLIKMAD
EQDCCLIVTIGGIGPAKRDVTPEATEAVCDRMMPGFGELMRAESLKEVPTAILSRQTAGLRGDSL
IVNLPGDPASISDCLLAVFPAIPYCIDLMEGPYLECNEAMIKPFRPKAK
T33-15B
(SEQ ID NO: 46)
(M)VRGIRGAITVNSDTPTSIIIATILLLEKMLEANGIQSYEELAAVIFTVTEDLTSAFPAEAAR
QIGMHRVPLLSAREVPVPGSLPRVIRVLALWNTDTPQDRVRHVYLSEAVRLRPDLESAQ
T33-21A
(SEQ ID NO: 47)
(M)RITTKVGDKGSTRLFGGEEVWKDSPIIEANGTLDELTSFIGEAKHYVDEEMKGILEEIQNDI
YKIMGEIGSKGKIEGISEERIAWLLKLILRYMEMVNLKSFVLPGGTLESAKLDVCRTIARRALRK
VLIVTREFGIGAEAAAYLLALSDLLFLLARVIEIEKNKLKEVRS
T33-21B
(SEQ ID NO: 48)
(M)PHLVIEATANLRLETSPGELLEQANKALFASGQFGEADIKSRFVTLEAYRQGTAAVERAYLH
ACLSILDGRDIATRTLLGASLCAVLAEAVAGGGEEGVQVSVEVREMERLSYAKRVVARQR
T33-28A
(SEQ ID NO: 49)
(M)ESVNTSFLSPSLVTIRDFDNGQFAVLRIGRTGFPADKGDIDLCLDKMIGVRAAQIFLGDDTE
DGFKGPHIRIRCVDIDDKHTYNAMVYVDLIVGTGASEVERETAEEEAKLALRVALQVDIADEHSC
VTQFEMKLREELLSSDSFHPDKDEYYKDFL
T33-28B
(SEQ ID NO: 50)
(M)PVIQTFVSTPLDHHKRLLLAIIYRIVTRVVLGKPEDLVMMTFHDSTPMHFFGSTDPVACVRV
EALGGYGPSEPEKVTSIVTAAITAVCGIVADRIFVLYFSPLHCGWNGTNF
T33-31A
(SEQ ID NO: 51)
(M)EEVVLITVPSALVAVKIAHALVEERLAACVNIVPGLTSIYREEGSVVSDHELLLLVKTTTDA
FPKLKERVKELHPYEVPEIVALPIAEGNREYLDWLRENTG

Table 1 provides the amino acid sequence of the first and second polypeptides; the right hand column in Table 1 identifies the residue numbers in each exemplary polypeptide that were identified as present at the interface of resulting assembled nanostructures (i.e.: “identified interface residues”). As can be seen, the number of interface residues for the exemplary polypeptides of SEQ ID NO:1-34 range from 4-13. In various embodiments, the first and second polypeptides comprise an amino acid sequence that is at least 75%, 80%, 85%, 90%, 91%, 92%, 93%, 94%, 95%, 96%, 97%, 98%, or 99% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. In other embodiments, the first and second polypeptides comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%, 60%, 70%, 75%, 80%, 90%, or 100% of the identified interface positions, to the amino acid sequence of a polypeptide selected from the group consisting of SEQ ID NOS:1-51.

As is the case with proteins in general, the polypeptides are expected to tolerate some variation in the designed sequences without disrupting subsequent assembly into nanostructures: particularly when such variation comprises conservative amino acid substitutions. As used here, “conservative amino acid substitution” means that: hydrophobic amino acids (Ala, Cys, Gly, Pro, Met, See, Sme, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

Table 2 lists surface amino acid residue numbers for each exemplary polypeptide of the invention denoted by SEQ ID NOS: 1-34. Thus, in various embodiments, 1 or more (at least 1, 2, 3, 4, 5, 6, 7, 8, 9, 10, 11, 12, 13, 14, 15, 16, 17, 18, 19, 20, 21, 22, 23, 24, 25, 26, 27 28, 29, 30, 31, 32, 33, 34, 35, 36, 37, 38, 39, 40, 41, 42, 43, 44, 45, 46, 47, 48, 49, 50, or more) of these surface residues may be modified in the polypeptides of the invention. Residues in parentheses are optional.

TABLE 2

| | | Surface residues not near |
|---|---|---|
| Name | Amino Acid Sequence | interface |
| I53-34A | (M)EGMDPLAVLAESRLLPLLTVRGGEDLAGLATVLELMGV | I53-34A: |
| SEQ ID | GALEITLRTEKGLEALKALRKSGLLLGAGTVRSPKEAEAAL | 6, 8, 9, 12, 14, 22, 25, 48, 49, 50, |
| NO: 1 | EAGAAFLVSPGLLEEVAALAQARGVPYLPGVLTPTEVERAL | 52, 53, 56, 73, 74, 81, 94, 95, 101, |
| | ALGLSALKFFPAEPFQGVRVLRAYAEVFPEVRFLPTGGIKE | 102, 103, |
| | EHLPHYAALPNLLAVGGSWLLQGDLAAVMKKVKAAKALLSP | 104, 119, 122, 137, 140, 143, 147, |
| | QAPG | 150, 151, 153, 161, 162, 163, 164, |
| | | 166, 167, 170, 172, 184, 193, 198, |
| | | 199, 200, 202 |
| I53-34B | (M)TKKVGIVDTTFARVDMAEAAIRTLKALSPNIKIIRKTV | I53-34B: |
| SEQ ID | PGIKDLPVACKKLLEEEGCDIVMALGMPGKAEKDKVCAHEA | 3, 12, 31, 33, 35, 36, 51, 54, 55, |
| NO: 2 | SLGLMLAQLMTNKHIIEVFVHEDEAKDDDELDILALVRAIE | 56, 59, 69, 70, 71, 74, 93, 103, 106, |
| | HAANVYYLLFKPEYLTRMAGKGLRQGREDAGPARE | 107, 108, 131, 132, 133, 134, |
| | | 138, 142, 153 |
| I53-40A | (M)TKKVGIVDTTFARVDMASAAILTLKMESPNIKIIRKTV | I53-40A: |
| SEQ ID | PGIKDLPVACKKLLEEEGCDIVMALGMPGKAEKDKVCAHEA | 3, 4, 31, 33, 35, 36, 37, 51, 54, 55, |
| NO: 3 | SLGLMLAQLMTNKHIIEVFVHEDEAKDDAELKILAARRAIE | 56, |
| | HALNVYYLFKPEYLTRMAGKGLRQGFEDAGPARE | 57, 59, 69, 70, 71, 74, 93, 103, 106, |
| | | 118, 127, 128, 131, 132, 133, 134, |
| | | 135, 138, 139, 142, 150, 153 |
| I53-40B | (M)STINNQLKALKVIPVIAIDNAEDIIPLGKVLAENGLPA | I53-40B: |
| SEQ ID | AEITFRSSAAVKAIMLLRSAQPEMLIGAGTILNGVQALAAK | 2, 3, 7, 9, 10, 12, 20, 21, 23, 26, |
| NO: 4 | EAGATFVVSPGFNPNTVRACQIIGIDIVPGVNNPSTVEAAL | 27, 30, |
| | EMGLTTLKFFPAEASGGISMVKSLVGPYGDIRLMPTGGITP | 34, 38, 45, 60, 62, 75, 85, 94, 95, |
| | SNIDNYLAIPQVLACGGTWMVDKKLVINGEWDEIARLTREI | 122, |
| | VEQVNP | 124, 126, 134, 139, 143, 151, 153, |
| | | 161, 163, 166, 167, 170, 172, 180, |
| | | 184, 185, 186, 189, 190, 192, 193, |
| | | 194, 195, 198, 201, 202, 205, 208, |
| | | 209 |
| I53-47A | (M)PIFTLNTNIKATDVPSDFLSLTSRLVGLILSKPGSYVA | I53-47A: |
| SEQ ID | VHINTDQQLSFGGSTNPAAFGTLMSIGGIEPSKNRDHSAVL | 11, 13, 14, 17, 34, 36, 37, 45, 47, |
| NO: 5 | FDHLNAMLGIPKNRMYIHFVNLNGDDVGWNGTTF | 54, 55, 56, 65, 69, 70, 71, 74, 91, |
| | | 92, 93, 101, 103, 105, 109, 110, |
| | | 112, 114 |
| I53-47B | (M)NQHSHKDYETVRIAVVRARWHADIVDACVEAFEIAMAA | I53-47B: |
| SEQ ID | IGGDRFAVDVFDVPGAYEIPLHARTLAETGRYGAVLGTAFV | 6, 7, 8, 9, 10, 11, 13, 18, 20, 21, |
| NO: 6 | VNGGIYRHEFVASAVIDGMMNVQLSTGVPVLSAVLTPHRYR | 24, 43, 44, 51, 63, 67, 70, 85, 87, |
| | DSAEHHRFFAAHFAVKGVEAARACIEILAAREKIAA | 101, 105, 122, 123, 124, 125, 126, |
| | | 147, 152, 153, 154 |
| I53-50A | (M)KMEELFKKHKIVAVLRANSVEEAIEKAVAVFAGGVHLI | I53-50A: |
| SEQ ID | EITFTVPDADTVIKALSVLKEKGAIIGAGTVTSVEQCRKAV | 4, 5, 6, 8, 9, 11, 17, 19, 23, 37, |
| NO: 7 | ESGAEFIVSPHLDEEISQFCKEKGVFYMPGVMTPTELVKAM | 46, 47, 59, 74, 77, 78, 81, 94, 95, |
| | KLGHTILKLFPGEVVGPQFVKAMKGPFPNVKFVPTGGVNLD | 98, 101, 102, |
| | NVCEWFKAGVLAVGVGSALVKGTPDEVREKAKAFVEKIRGC | 103, 106, 119, 122, 126, 139, 142, |
| | TE | 145, 149, 150, 152, 160, 161, |
| | | 162, 163, 166, 169, 179, 183, |
| | | 185, 188, 191, 192, 194, 198, |
| | | 199 |
| I53-50B | (M)NQHSHKDYETVRIAVVRARWHAEIVDACVSAFEAAMAD | I53-50B: |
| SEQ ID | IGGDRFAVDVFDVPGAYEIPLHARTLAETGRYGAVLGTAFV | 6, 7, 8, 9, 10, 11, 13, 18, 20, 21, |
| NO: 8 | VNGGIYRHEFVASAVIDGMMNVQLSTGVPVLSAVLTPHRYR | 34, 38, 39, 40, 43, 44, 48, 51, 63, |
| | DSDAHILLFLALFAVKGMEAARACVEILAAREKIAA | 67, 70, 87, 101, 105, 118, 143, |
| | | 147, 152, 153, 154 |
| I53-51A | (M)FIKSGDDGNINVINKRVGKDSPLVNFLGDLDELNSFIG | I53-51A: |
| SEQ ID | FAISKIPWEDMKKDLERVQVELFEIGEDLSTQSSKKKIDES | 19, 20, 24, 28, 46, 47, 51, 70, 71, |
| NO: 9 | YVLWLLAATAIYRIESGPVKLFVIPGGSEEASVLHVTRSVA | 73, 74, 75, 76, 102, 122, 130, 133, |
| | RRVERNAVKYTKELPEINRMIIVYLNRLSSLLFAMALVANK | 134, 135, 136, 137, 140, 162, |
| | RRNQSEKIYEIGKSW | 163, 164, 165, |
| | | 169, 175, 177 |
| I53-51B | (M)NQHSHKDYETVRIAVVRARWHADIVDQCVRAFEEAMAD | I53-51B: |
| SEQ ID | AGGDRFAVDVFDVPGAYEIPLHARTLAETGRYGAVLGTAFV | 6, 7, 8, 9, 10, 11, 13, 18, 21, 27, |
| NO: 10 | VNGGIYRHEFVASAVIDGMMNVQLSTGVPVLSAVLTPHRYR | 34, 38, |
| | SSREHHEFFREHFMVKGVEAAAACITILAAREKIAA | 43, 48, 63, 67, 70, 85, 87, 101, |
| | | 118, |
| | | 125, 126, 129, 152, 153, 154 |
| I52-03A | (M)GHTKGPTPQQHDGSALRIGIVHARWNKTIIMPLLIGTI | I52-03A: |
| SEQ ID | AKLLECGVKASNIVVQSVPGSWELPIAVQRLYSASQLQTPS | 6, 9, 10, 11, 13, 15, 16, 26, 48, |
| NO: 11 | SGPSLSAGDLLGSSTTDLTALPTTTASSTGPFDALIAIGVL | 69, 75, |
| | IKGETMHFEYIADSVSHGLMRVQLDTGVPVIFGVLTVLTDD | 76, 78, 79, 111, 125, 127, 142, |
| | QAKARAGVIEGSHNHGEDWGLAAVEMGVRRRDWAAGKTE | 146, |
| | | 159, 160, 161, 162, 171, 175, 193, |
| | | 194, 196, 197, 199, 200 |
| I52-03B | (M)YEVDHADVYDLFYLGRGKDYAAEASDIADLVRSRTPEA | I52-03B: |
| SEQ ID | SSLLDVACGTGTHLEHFTKEFGDTAGLELSEDMLTHARKRL | 2, 3, 5, 6, 8, 15, 17, 20, 22, 23, |
| NO: 12 | PDATLHQGDMRDFQLGRKFSAVVSMFSSVGYLKTVAELGAA | 26, 27, |
| | VASFAEHLEPGGVVVVEPWWFPETFADGWVSADVVRRDGRT | 30, 33, 34, 35, 37, 38, 40, 54, 55, |
| | VARVSHSVREGNATRMEVHFTVADPGKGVRHFSDVHLITLF | 57, 58, 59, 61, 62, 68, 70, 71, 74, |
| | HQREYEAAFMAAGLRVEYLEGGPSGRGLFVGVPA | 77, 78, 79, |
| | | 81, 82, 84, 86, 87, 91, 96, 97, 98, |
| | | 111, |
| | | 127, 130, 131, 132, 141, 144, 145, |
| | | 148, 150, 154, 157, 158, 159, 160, |
| | | 161, 171, 172, 173, 174, 177, 187, |
| | | 189, 192, 198, 199, 222, 223, 224, |
| | | 236 |
| I52-32A | (M)GMKEKFVLIITHGDFGKGLLSGAEVIIGKQENVHTVGL | I52-32A: |
| SEQ ID | NLGDNIEKVAKEVMRIIIAKLAEDKEIIIVVDLFGGSPFNI | 3, 5, 15, 18, 30, 32, 35, 40, 41, |
| NO: 13 | ALEMMKTFDVKVITGINMPMLVELLTSINVYDTTELLENIS | 42, 44, |
| | KIGKDGIKVIEKSSLKM | 45, 65, 73, 79, 91, 103, 106, 109, |
| | | 110, 111, 112, 114, 115, 118, |
| | | 122, 123, 125, 126, 129, 131 |
| I52-32B | (M)KYDGSKLRIGILHARWNLEIIAALVAGAIKRLQEFGVK | I52-32B: |
| SEQ ID | AENIIIETVPGSFELPYGSKLFVEKQKRLGKPLDAIIPIGV | 4, 6, 7, 9, 17, 32, 35, 42, 59, 63, |
| NO: 14 | LIKGSTMHFEYICDSTTHQLMKLNFELGIPVIFGVLTCLTD | 64, 66, |
| | EQAEARAGLIEGKMHNHGEDWGAAAVEMATKFN | 67, 68, 69, 70, 71, 73, 83, 85, 90, |
| | | 106, |
| | | 119, 120, 121, 122, 125, 131, 133, |
| | | 134, 135, 136, 154 |
| I52-33A | (M)AVKGLGEVDQKYDGSKLRIGILHARWNRKIILALVAGA | I52-33A: |
| SEQ ID | VLRLLEFGVKAENIIIETVPGSFELPYGSKLFVEKQKRLGK | 12, 14, 16, 17, 19, 26, 27, 46, 69 |
| NO: 15 | PLDAIIPIGVLIKGSTMHFEYICDSTTHQLMKLNFELGIPV | 73, 74, 76, 77, 78, 80, 81, 83, 93, |
| | IFGVLTCLTDEQAEARAGLIEGKMHNHGEDWGAAAVEMATK | 95, 100, 116, 129, 130, 131, |
| | FN | 132, 145, 164 |
| I52-33B | (M)GANWYLDNESSRLSFTSTKNADIAEVHRFLVLHGKVDP | I52-33B: |
| SEQ ID | KGLAEVEVETESISTGIPLRDMLLRVLVFQVSKFPVAQINA | 4, 6, 10, 20, 21, 23, 24, 31, 32, |
| NO: 16 | QLDMRPINNLAPGAQLELRLPLTVSLRGKSHSYNAELLATR | 34, 36, |
| | LDERRFQVVTLEPLVIHAQDFDMVRAFNALRLVAGLSAVSL | 39, 40, 42, 44, 46, 48, 56, 73, 77, |
| | SVPVGAVLIFTAR | 81, 83, 85, 88, 89, 91, 92, 96, 97, |
| | | 99, |
| | | 101, 103, 109, 110, 111, 112, 114, |
| | | 124, 125, 138, 140, 143, 158, 175 |
| I32-06A | (M)TDYIRDGSAIKALSFAIILAEADLRHIPQDLQRLAVRV | I32-06A: |
| SEQ ID | IHACGMVDVANDLAFSEGAGKAGRNALLAGAPILCDARMVA | 24, 26, 27, 41, 47, 50, 51, 56, 60, |
| NO: 17 | EGITRSRLPADNRVIYTLSDPSVPELAKKIGNTRSAAALDL | 63, 64, 67, 68, 77, 84, 85, 86, 91, |
| | WLPHIEGSIVAIGNAPTALFRLFELLDAGAPKPALIIGMPV | 93, 98, 99, |
| | GFVGAAESKDELAANSRGVPYVIVRGRRGGSAMTAAAVNAL | 100, 101, 102, 105, 108, 109, 114, |
| | ASERE | 123, 124, 125, 127, 135, 142, 145, |
| | | 148, 149, 152, 153, 169, 172, 173, |
| | | 176, 177, 180, 187, 189 |
| I32-06B | (M)ITVFGLKSKLAPRREKLAEVIYSSLHLGLDIPKGKHAI | I32-06B: |
| SEQ ID | RFLCLEKEDFYYPFDRSDDYTVIEINLMAGRSEETKMLLIF | 8, 9, 10, 13, 14, 15, 16, 17, 20, |
| NO: 18 | LLFIALERKLGIRAHDVEITIKEQPAHCWGFRGRTGDSARD | 34, 36, |
| | LDYDIYV | 45, 46, 47, 50, 51, 53, 54, 57, 67, |
| | | 70, 91, 93, 95, 105, 112 |
| I32-19A | (M)GSDLQKLQRFSTCDISDGLLNVYNIPIGGYFPNLTAIS | I32-19A: |
| SEQ ID | PPQNSSIVGTAYTVLFAPIDDPRPAVNYIDSVPPNSILVLA | 3, 4, 6, 7, 9, 10, 25, 27, 36, 40, |
| NO: 19 | LEPHLQSQFHPFIKITQAMYGGLMSTRAQYLKSNGTVVFGR | 42, 43, 44, 49, 58, 59, 61, 62, 63, |
| | IRDVDEHRTLNHPVFAYGVGSCAPKAVVKAVGTNVQLKILT | 70, 72, 73, 74, |
| | SDGVTQTICPGDYIAGDNNGIVRIPVQETDISKLVTYIEKS | 82, 84, 88, 89, 109, 110, 112, 126, |
| | IEVDRLVSEAIKNGLPAKAAQTARRMVLKDYI | 127, 129, 130, 132, 146, 155, |
| | | 156, 157, |
| | | 159, 166, 169, 172, 189, 190, 192, |
| | | 194, 195, 198, 201, 204, 215, 232 |
| I32-19B | (M)SGMRVYLGADHAGYELKQAIIAFLKMTGHEPIDCGALR | I32-19B: |
| SEQ ID | YDADDDYPAFCIAAATRTVADPGSLGIVLGGSGNGEQIAAN | 4, 5, 31, 33, 38, 41, 42, 43, 55, |
| NO: 20 | KVPGARCALAWSVQTAALAREHNNAQLIGIGGRMHTLEEAL | 56, 59, |
| | RIVKAFVTTPWSKAQRHQRRIDILAEYERTHEAPPVPGAPA | 61, 62, 83, 93, 94, 101, 104, 113, |
| | | 119, 129, 131, 134, 136, 137, |
| | | 139, 140, |
| | | 143, 144, 146, 147, 150, 152, 153, |
| | | 156, 158, 159 |
| I32-28A | (M)GDDARIAAIGDVDELNSQIGVLLAEPLPDDVRAALSAI | I32-28A: |
| SEQ ID | QHDLFDLGGELCIPGHAAITEDHLLRLALWLVHYNGQLPPL | 4, 6, 7, 10, 14, 27, 30, 31, 33, 34, |
| NO: 21 | EEFILPGGARGAALAHVCRTVCRRAERSIKALGASEPLNIA | 41, 44, 45, 51, 52, 53, 54, 55, 56, |
| | PAAYVNLLSDLLFVLARVLNRAAGGADVLWDRTRAH | 59, 76, 78, 79, 80, 81, 82, 83, 90, |
| | | 103, 111, 115, 116, 131, 134, |
| | | 142, 145, 147, 150 |
| I32-28B | (M)ILSAEQSFTLRHPHGQAAALAFVREPAAALAGVQRLRG | I32-28B: |
| SEQ ID | LDSDGEQVWGELLVRVPLLGEVDLPFRSEIVRTPQGAELRP | 3, 4, 6, 8, 12, 15, 17, 18, 22, 26, |
| NO: 22 | LTLTGERAWVAVSGQATAAEGGEMAFAFQFQAHLATPEAEG | 28, 32, |
| | EGGAAFEVMVQAAAGVTLLLVAMALPQGLAAGLPPA | 38, 39, 41, 43, 45, 46, 48, 50, 60, |
| | | 66, 68, 71, 73, 74, 79, 81, 82, 83, |
| | | 84, 86, 87, |
| | | 95, 100, 103, 105, 109, 111, 113, |
| | | 151, 152, 155, 156, 157 |
| I53- | (M)TKKVGIVDTTFARVDMASAAILTLKMESPNIKIIRKTV | I53-40A: |
| 40A. 1 | PGIKDLPVACKKLLEEEGCDIVMALGMPGKKEKDKVCAHEA | 3, 4, 31, 33, 35, 36, 37, 51, 54, |
| SEQ ID | SLGLMLAQLMTNKHIIEVFVHEDEAKDDAELKILAARRAIE | 55, 56, |
| NO: 23 | HALNVYYLLFKPEYLTRMAGKGLRQGFEDAGPARE | 57, 59, 69, 70, 71, 74, 93, 103, |
| | | 106, |
| | | 118, 127, 128, 131, 132, 133, 134, |
| | | 135, 138, 139, 142, 150, 153 |
| I53- | (M)DDINNQLKRLKVIPVIAIDNAEDIIPLGKVLAENGLPA | I53-40B: |
| 40B.1 | AEITFRSSAAVKAIMLLRSAQPEMLIGAGTILNGVQALAAK | 2, 3, 7, 9, 10, 12, 20, 21, 23, 26, |
| SEQ ID | EAGADFVVSPGFNPNTVRACQIIGIDIVPGVNNPSTVEQAL | 27, 30, |
| NO: 24 | EMGLTTLKFFPAEASGGISMVKSLVGPYGDIRLMPTGGITP | 34, 38, 45, 60, 62, 75, 85, 94, 95, |
| | DNIDNYLAIPQVLACGGTWMVDKKLVRNGEWDEIARLTREI | 122, |
| | VEQVNP | 124, 126, 134, 139, 143, 151, 153, |
| | | 161, 163, 166, 167, 170, 172, 180, |
| | | 184, 185, 186, 189, 190, 192, 193, |
| | | 194, 195, 198, 201, 202, 205, 208, |
| | | 209 |
| I53- | (M)PIFTLNTNIKADDVPSDFLSLTSRLVGLILSKPGSYVA | I53-47A: |
| 47A.1 | VHINTDQQLSFGGSTNPAAFGTLMSIGGIEPDKNRDHSAVL | 11, 13, 14, 17, 34, 36, 37, 45, 47, |
| SEQ ID | FDHLNAMLGIPKNRMYIHFVNLNGDDVGWNGTTF | 54, 55, 56, 65, 69, 70, 71, 74, 91, |
| NO: 25 | | 92, 93, 101, 103, 105, 109, 110, |
| | | 112, 114 |
| I53- | (M)PIFTLNTNIKADDVPSDFLSLTSRLVGLILSEPGSYVA | I53-47A: |
| 47A.1NegT2 | VHINTDQQLSFGGSTNPAAFGTLMSIGGIEPDKNEDHSAVL | 11, 13, 14, 17, 34, 36, 37, 45, 47, |
| SEQ ID | FDHLNAMLGIPKNRMYIHFVDLDGDDVGWNGTTF | 54, 55, 56, 65, 69, 70, 71, 74, 91, |
| NO: 26 | | 92, 93, 101, 103, 105, 109, 110, |
| | | 112, 114 |
| I53- | (M)NQHSHKDHETVRIAVVRARWHADIVDACVEAFEIAMAA | I53-47B: |
| 47B.1 | IGGDRFAVDVEDVPGAYEIPLHARTLAETGRYGAVLGTAFV | 6, 7, 8, 9, 10, 11, 13, 18, 20, 21, |
| SEQ ID | VNGGIYRHEFVASAVIDGMMNVQLDTGVPVLSAVLTPHRYR | 24, 43, 44, 51, 63, 67, 70, 85, 87, |
| NO: 27 | DSDEHHRFFAAHFAVKGVEAARACIEILNAREKIAA | 101, 105, 122, 123, 124, 125, 126, |
| | | 147, 152, 153, 154 |
| I53- | (M)NQHSHKDHETVRIAVVRARWHADIVDACVEAFEIAMAA | I53-47B: |
| 47B.1NegT2 | IGGDRFAVDVEDVPGAYEIPLHARTLAETGRYGAVLGTAFV | 6, 7, 8, 9, 10, 11, 13, 18, 20, 21, |
| SEQ ID | VDGGIYDHEFVASAVIDGMMNVQLDTGVPVLSAVLTPHEYE | 24, 43, 44, 51, 63, 67, 70, 85, 87, |
| NO: 28 | DSDEDHEFFAAHFAVKGVEAARACIEILNAREKIAA | 101, 105, 122, 123, 124, 125, 126, |
| | | 147, 152, 153, 154 |
| I53- | (M)KMEELFKKHKIVAVLRANSVEEAIEKAVAVFAGGVHLI | I53-50A: |
| 50A.1 | EITFTVPDADTVIKALSVLKEKGAIIGAGTVTSVEQCRKAV | 4, 5, 6, 8, 9, 11, 17, 19, 23, 37, |
| SEQ ID | ESGAEFIVSPHLDEEISQFCKEKGVFYMPGVMTPTELVKAM | 46, 47, 59, 74, 77, 78, 81, 94, 95, |
| NO: 29 | KLGHDILKLFPGEVVGPQFVKAMKGPFPNVKFVPTGGVNLD | 98, 101, 102, 103, 106, 119, 122, |
| | NVCEWFKAGVLAVGVGDALVKGDPDEVREKAKKFVEKIRGC | 126, 139, 142, 145, 149, 150, |
| | TE | 152, 160, 161, 162, 163, 166, 169, |
| | | 179, 183, 185, 188, 191, 192, |
| | | 194, 198, 199 |
| I53- | (M)KMEELFKKHKIVAVLRANSVEEAIEKAVAVFAGGVHLI | I53-50A: |
| 50A.1NegT2 | EITFTVPDADTVIKALSVLKEKGAIIGAGTVTSVEQCRKAV | 4, 5, 6, 8, 9, 11, 17, 19, 23, 37, |
| SEQ ID | ESGAEFIVSPHLDEEISQFCKEKGVFYMPGVMTPTELVKAM | 46, 47, 59, 74, 77, 78, 81, 94, 95, |
| NO: 30 | KLGHDILKLFPGEVVGPEFVEAMKGPFPNVKFVPTGGVDLD | 98, 101, 102, 103, 106, 119, 122, |
| | DVCEWFDAGVLAVGVGDALVEGDPDEVREDAKEFVEEIRGC | 126, 139, 142, 145, 149, 150, |
| | TE | 152, 160, 161, 162, 163, 166, 169, |
| | | 179, 183, 185, 188, 191, 192, |
| | | 194, 198, 199 |
| I53- | (M)KMEELFKKHKIVAVLRANSVEEAIEKAVAVFAGGVHLI | I53-50A: |
| 50A.1PosT1 | EITFTVPDADTVIKALSVLKEKGAIIGAGTVTSVEQCRKAV | 4, 5, 6, 8, 9, 11, 17, 19, 23, 37, |
| SEQ ID | ESGAEFIVSPHLDEEISQFCKEKGVFYMPGVMTPTELVKAM | 46, 47, 59, 74, 77, 78, 81, 94, 95, |
| NO: 31 | KLGHDILKLFPGEVVGPQFVKAMKGPFPNVKFVPTGGVNLD | 98, 101, 102, 103, 106, 119, 122, |
| | NVCKWFKAGVLAVGVGKALVKGKPDEVREKAKKFVKKIRGC | 126, 139, 142, 145, 149, 150, |
| | TE | 152, 160, 161, 162, 163, 166, 169, |
| | | 179, 183, 185, 188, 191, 192, |
| | | 194, 198, 199 |
| I53- | (M)NQHSHKDHETVRIAVVRARWHAEIVDACVSAFEAAMRD | I53-50B: |
| 50B.1 | IGGDRFAVDVEDVPGAYEIPLHARTLAETGRYGAVLGTAFV | 6, 7, 8, 9, 10, 11, 13, 18, 20, 21, |
| SEQ ID | VNGGIYRHEFVASAVIDGMMNVQLDTGVPVLSAVLTPHRYR | 34, 38, 39, 40, 43, 44, 48, 51, 63, |
| NO: 32 | DSDAHILLFLALFAVKGMEAARACVEILAAREKIAA | 67, 70, 87, 101, 105, 118, 143, |
| | | 147, 152, 153, 154 |
| I53- | (M)NQHSHKDHETVRIAVVRARWHAEIVDACVSAFEAAMRD | I53-50B: |
| 50B.1NegT2 | IGGDRFAVDVEDVPGAYEIPLHARTLAETGRYGAVLGTAFV | 6, 7, 8, 9, 10, 11, 13, 18, 20, 21, |
| SEQ ID | VDGGIYDHEFVASAVIDGMMNVQLDTGVPVLSAVLTPHEYE | 34, 38, 39, 40, 43, 44, 48, 51, 63, |
| NO: 33 | DSDADTLLFLALFAVKGMEAARACVEILAAREKIAA | 67, 70, 87, 101, 105, 118, 143, |
| | | 147, 152, 153, 154 |
| I53- | (M)NQHSHKDHETVRIAVVRARWHAEIVDACVSAFEAAMRD | I53-50B: |
| 50B.4PosT1 | IGGDRFAVDVEDVPGAYEIPLHARTLAETGRYGAVLGTAFV | 6, 7, 8, 9, 10, 11, 13, 18, 20, 21, |
| SEQ ID | VNGGIYRHEEVASAVINGMMNVQLNTGVPVLSAVLTPHNYD | 34, 38, 39, 40, 43, 44, 48, 51, 63, |
| NO: 34 | KSKAHTLLFLALFAVKGMEAARACVEILAAREKIAA | 67, 70, 87, 101, 105, 118, 143, |
| | | 147, 152, 153, 154 |

In various embodiments of the nanostructure of the invention, the first polypeptides and the second polypeptides comprise polypeptides with the amino acid sequence selected from the following pairs, or modified versions thereof (i.e.: permissible modifications as disclosed for the polypeptides of the invention: isolated polypeptides comprising an amino acid sequence that is at least 75% 80%, 85%, 90%, 91%, 92%, 93%, 94%, 95%, 96%, 97%, 98%, 99%, or 100% identical over its length, and/or identical at least at one identified interface position, to the amino acid sequence indicated by the SEQ ID NO.):
SEQ ID NO:1 and SEQ ID NO:2 (I53-34A and I53-34B);
SEQ ID NO:3 and SEQ ID NO:4 (I53-40A and I53-40B);
SEQ ID NO:3 and SEQ ID NO:24 (I53-40A and I53-40B.1);
SEQ ID NO:23 and SEQ ID NO:4 (I53-40A.1 and I53-40B);
SEQ ID NO:35 and SEQ ID NO:36 (I53-40A genus and I53-40B genus);
SEQ ID NO:5 and SEQ ID NO:6 (I53-47A and I53-47B);
SEQ ID NO:5 and SEQ ID NO:27 (I53-47A and I53-47B.1);
SEQ ID NO:5 and SEQ ID NO:28 (I53-47A and I53-47B.1NegT2);
SEQ ID NO:25 and SEQ ID NO:6 (I53-47A.1 and I53-47B);
SEQ ID NO:25 and SEQ ID NO:27 (I53-47A.1 and I53-47B.1);
SEQ ID NO:25 and SEQ ID NO:28 (I53-47A.1 and I53-47B.1NegT2);
SEQ ID NO:26 and SEQ ID NO:6 (I53-47A.1NegT2 and I53-47B);
SEQ ID NO:26 and SEQ ID NO:27 (I53-47A.1NegT2 and I53-47B.1);
SEQ ID NO:26 and SEQ ID NO:28 (I53-47A.1NegT2 and I53-47B.1NegT2);
SEQ ID NO:37 and SEQ ID NO:38 (I53-47A genus and I53-47B genus);
SEQ ID NO:7 and SEQ ID NO:8 (I53-50A and I53-50B);
SEQ ID NO:7 and SEQ ID NO:32 (I53-50A and I53-50B.1);
SEQ ID NO:7 and SEQ ID NO:33 (I53-50A and I53-50B.1NegT2);
SEQ ID NO:7 and SEQ ID NO:34 (I53-50A and I53-50B.4PosT1);
SEQ ID NO:29 and SEQ ID NO:8 (I53-50A.1 and I53-50B);
SEQ ID NO:29 and SEQ ID NO:32 (I53-50A.1 and I53-50B.1);
SEQ ID NO:29 and SEQ ID NO:33 (I53-50A.1 and I53-50B.1NegT2);
SEQ ID NO:29 and SEQ ID NO:34 (I53-50A.1 and I53-50B.4PosT1);
SEQ ID NO:30 and SEQ ID NO:8 (I53-50A.1NegT2 and I53-50B);
SEQ ID NO:30 and SEQ ID NO:32 (I53-50A.1NegT2 and I53-50B.1);
SEQ ID NO:30 and SEQ ID NO:33 (I53-50A.1NegT2 and I53-50B.1NegT2);
SEQ ID NO:30 and SEQ ID NO:34 (I53-50A.1NegT2 and I53-50B.4PosT1);
SEQ ID NO:31 and SEQ ID NO:8 (I53-50A.1PosT1 and I53-50B);
SEQ ID NO:31 and SEQ ID NO:32 (I53-50A.1PosT1 and I53-50B.1);
SEQ ID NO:31 and SEQ ID NO:33 (I53-50A.1PosT1 and I53-50B.1NegT2);
SEQ ID NO:31 and SEQ ID NO:34 (I53-50A.1PosT1 and I53-50B.4PosT1);
SEQ ID NO:39 and SEQ ID NO:40 (I53-50A genus and I53-50B genus);
SEQ ID NO:9 and SEQ ID NO:10 (I53-51A and I53-51B);
SEQ ID NO:11 and SEQ ID NO:12 (I52-03A and I52-03B);
SEQ ID NO:13 and SEQ ID NO:14 (I52-32A and I52-32B);
SEQ ID NO:15 and SEQ ID NO:16 (I52-33A and I52-33B)
SEQ ID NO:17 and SEQ ID NO:18 (I32-06A and I32-06B);
SEQ ID NO:19 and SEQ ID NO:20 (I32-19A and I32-19B);
SEQ ID NO:21 and SEQ ID NO:22 (I32-28A and I32-28B);
SEQ ID NO:23 and SEQ ID NO:24 (I53-40A.1 and I53-40B.1);
SEQ ID NO:41 and SEQ ID NO:42 (T32-28A and T32-28B);
SEQ ID NO:43 and SEQ ID NO:44 (T33-09A and T33-09B);
SEQ ID NO:45 and SEQ ID NO:46 (T33-15A and T33-15B);
SEQ ID NO:47 and SEQ ID NO:48 (T33-21A and T33-21B);
SEQ ID NO:49 and SEQ ID NO:50 (T33-28A and T32-28B); and
SEQ ID NO:51 and SEQ ID NO:44 (T33-31A and T33-09B (also referred to as T33-31B))

In one embodiment, the one or more paramyxovirus and/or pneumovirus F proteins, or antigenic fragments thereof, are expressed as a fusion protein with the first and/or second polypeptides. In these embodiments, it is preferred that the one or more paramyxovirus and/or pneumovirus F proteins, or antigenic fragments thereof are present at the N terminus of the fusion protein, whenever this configuration can facilitate presentation of the one or more paramyxovirus and/or pneumovirus F proteins, or antigenic fragments thereof on an exterior of the nanostructure. This preference for the presence of the paramyxovirus and/or pneumovirus F protein at the N terminus of the fusion protein derives from the location of the C terminus of the paramyxovirus and/or pneumovirus F proteins at one extreme (the “bottom”) of the F protein trimer; by locating the genetic fusion at this point, the majority of the F protein structure will be displayed and accessible on the nanostructure exterior. In a further embodiment, the nanostructures comprise one or more copies of a fusion protein comprising at least two domains—a paramyxovirus and/or pneumovirus F protein, or an antigenic fragment thereof, and a trimeric assembly domain (i.e.: each first assembly is a homotrimer of the first polypeptide)—and one or more copies of a second oligomeric block (i.e.: each second assembly is an oligomer of two or more copies of the second polypeptide). In another embodiment, the first and or second polypeptides may be modified to permit the one or more paramyxovirus and/or pneumovirus F proteins, or antigenic fragments thereof, to be covalently linked to the first and/or second polypeptides. In one non-limiting example, the first and/or second polypeptides can be modified, such as by introduction of various cysteine residues at defined positions to facilitate linkage one or more paramyxovirus and/or pneumovirus F proteins, or antigenic fragments thereof.

In other embodiments, the one or more paramyxovirus and/or pneumovirus F proteins, or antigenic fragments thereof are attached to the first or second polypeptides via any suitable technique, including but not limited to covalent chemical cross-linking (via any suitable cross-linking technique) and non-covalent attachment including engineered electrostatic interactions.

### Trimeric Assembly Domains

In one embodiment of a trimeric assembly that comprises a trimeric paramyxovirus and/or pneumovirus F protein, or antigenic fragments thereof, the paramyxovirus and/or pneumovirus F protein, or antigenic fragment thereof is genetically fused to the first polypeptides that self-assemble into the trimeric assembly. The trimeric assembly comprises a protein-protein interface that induces three copies of the first polypeptides to self-associate to form trimeric building blocks. Each copy of the first polypeptides further comprises a surface-exposed interface that interacts with a complementary surface-exposed interface on a second assembly domain. As described in King et al. (Nature 510, 103-108, 2014), Bale et al. (Science 353, 389-394, 2016), and patent publications WO2014124301 A1 and US20160122392 A1, the complementary protein-protein interface between the trimeric assembly domain and second assembly domain drives the assembly of multiple copies of the trimeric assembly domain and second assembly domain to a target nanostructure. In some embodiments, each copy of the trimeric assembly domains of the nanostructure bears a paramyxovirus and/or pneumovirus F proteins, or antigenic fragment thereof, as a genetic fusion; these nanostructures display the F proteins at full valency. In other embodiments, the nanostructures of the invention comprise one or more copies of trimeric assembly domains bearing paramyxovirus and/or pneumovirus F proteins, or antigenic fragments thereof as genetic fusions as well as one or more trimeric assembly domains that do not bear F proteins as genetic fusions; these nanostructures display the F proteins at partial valency. The trimeric assembly domain can be any polypeptide sequence that forms a trimer and interacts with a second assembly domain to drive assembly to a target nanostructure.

In one specific embodiment, the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-31A (SEQ ID NO:51) and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-09B/T33-31B (SEQ ID NO:44) (residues in parentheses are optional)

T33-31A
(SEQ ID NO: 51)
(M)EEVVLITVPSALVAVKIAHALVEERLAACVNIVPGLTSIYREEGSV
VSDHELLLLVKITTDAFPKLKERVKELHPYEVPEIVALPIAEGNREYLD
WLRENTG
>T33-31B
(SEQ ID NO: 44)
(M)VRGIRGAITVEEDTPAAILAATIELLLKMLEANGIQSYEELAAVIF
TVTEDLTSAFPAEAARLIGMHRVPLLSAREVPVPGSLPRVIRVLALWNT
DTPQDRVRHVYLNEAVRLRPDLESAQ

In another specific embodiment, the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-15A (SEQ ID NO:45) and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-15B (SEQ ID NO:46).

In various further specific embodiments, the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of a polypeptides selected from the group consisting of I53-50A (SEQ ID NO:7), I53-50A.1 (SEQ ID NO:29), I53-50A.1NegT2 (SEQ ID NO:30), and I53-50A.1PosT1 (SEQ ID NO:31), and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of a polypeptide selected from the group consisting of I53-50B (SEQ ID NO:8), I53-50B.1 (SEQ ID NO:32), I53-50B.1NegT2 (SEQ ID NO:33), and I53-50B.4PosT1 (SEQ ID NO:34).

In another specific embodiment, the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of I32-28A (SEQ ID NO:21) and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of I32-28B (SEQ ID NO:22).

The nanostructures of the invention display multiple copies (i.e.: 2, 3, or more) of one or more paramyxovirus and/or pneumovirus F proteins, or antigenic fragments thereof, on an exterior of the nanostructure. Exemplary paramyxovirus and/or pneumovirus include, but are not limited to, respiratory syncytial virus (RSV) and Human metapneumovirus (hMPV). (C. L. Afonso et al., Taxonomy of the order Mononegavirales: update 2016. Arch. Virol. 161, 2351-2360 (2016)).

As used herein, “on an exterior of the nanostructure” means that an antigenic portion of the one or more paramyxovirus and/or pneumovirus F proteins, or antigenic fragments thereof, must be accessible for binding by B cell receptors, antibodies, or antibody fragments and not buried within the nanostructure.

The one or more paramyxovirus and/or pneumovirus F proteins, or antigenic fragments thereof, may comprise any suitable native F proteins, post-fusion, or pre-fusion (preF) antigens, or mutants thereof capable of inducing an immune response that will generate antibodies that bind to paramyxovirus and/or pneumovirus F proteins. A nanostructure may display more than one F protein; thus, in some embodiments the one or more paramyxovirus and/or pneumovirus F proteins, or antigenic fragments thereof comprise 1, 2, 3, 4, or more F proteins or antigenic fragments thereof. In one embodiment, the one or more paramyxovirus and/or pneumovirus F proteins, or antigenic fragments thereof may be as defined in patent publication number US 2016/0046675 A1. In some embodiments, the one or more paramyxovirus and/or pneumovirus F proteins, or antigenic fragments thereof, are selected from the group consisting of SEQ ID NOS: 1-350, 370-382, 389-693, 698-1026, 1429-1442, 1456-1468, and 1474-1478 as disclosed in US published patent application 2016/0046675. In other embodiments, the one or more paramyxovirus and/or pneumovirus F proteins, or antigenic fragments thereof may be as defined in WO2012158613, US 20160102123, US20140141037, WO2014079842, WO2014160463, US20140271699, EP2970393, WO2014174018, US20140271699, US20160176932, US20160122398, WO2017040387, WO2017109629, WO2017172890, WO2017207477, Krarup et al. (2015) Nature Communications 6:8143, and WO2017207480.

In a specific embodiment, the one or more paramyxovirus and/or pneumovirus F proteins, or antigenic fragments thereof, comprise a polypeptide having at least 75%, 80%, 85%, 90%, 91%, 92%, 93%, 94%, 95%, 96%, 97%, 98%, 99%, or 100% identity along its full length to the amino acid sequence of DS-Cav1 shown below (residues in parentheses are optional; note that the N-terminal residues in parentheses are cleaved from the protein during secretion—the mature N terminus begins with QNITEEF . . . (SEQ ID NO:52)). DS-Cav1 comprises a prefusion-stabilized form of the fusion (F) glycoprotein, which elicits improved protective responses against respiratory syncytial virus (RSV) in mice and macaques compared to postfusion RSV F (McLellan et al. (2013) Science 342:592-8).

DS-Cav1 (SEQ ID NO: 53):
(MELLILKANAITTILTAVTFCFASG)QNITEEFYQSTCSAVSKGYLSA
LRTGWYTSVITIELSNIKENKCNGTDAKVKLIKQELDKYKNAVTELQLL
MQSTPATNNRARRELPRFMNYTLNNAKKTNVTLSKKRKRRFLGFLLGVG
SAIASGVAVCKVLHLEGEVNKIKSALLSTNKAVVSLSNGVSVLTFKVLD
LKNYIDKQLLPILNKQSCSISNIETVIEFQQKNNRLLEITREFSVNAGV
TTPVSTYMLTNSELLSLINDMPITNDQKKLMSNNVQIVRQQSYSIMCII
KEEVLAYVVQLPLYGVIDTPCWKLHTSPLCTINTKEGSNICLTRTDRGW
YCDNAGSVSFFPQAETCKVQSNRVFCDTMNSLTLPSEVNLCNVDIFNPK
YDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEF
DASISQVNEKINQSLAFIR(KSDELL)

In other embodiments, the F protein may comprise a polypeptide having at least 75%, 80%, 85%, 90%, 91%, 92%, 93%, 94%, 95%, 96%, 97%, 98%, 99%, or 100% identity along its full length to a polypeptide selected from:

RSV F
sc9-10 DS-Cav1 A149C Y458C
(SEQ ID NO: 61)
(MELLILKANAITTILTAVTFCFASG)QNITEEFYQSTCSAVSKGYLSA
LRTGWYTSVITIELSNIKENKCNGTDAKVKLIKQELDKYKNAVTELQLL
MQSTPATGSGSAICSGVAVCKVLHLEGEVNKIKSALLSTNKAVVSLSNG
VSVLTFKVLDLKNYIDKQLLPILNKQSCSISNIETVIEFQQKNNRLLEI
TREFSVNAGVTTPVSTYMLTNSELLSLINDMPITNDQKKLMSNNVQIVR
QQSYSIMCIIKEEVLAYVVQLPLYGVIDTPCWKLHTSPLCTTNTKEGSN
ICLTRTDRGWYCDNAGSVSFFPQAETCKVQSNRVFCDTMNSRTLPSEVN
LCNVDIFNPKYDCKIMTSKTDVSSSVITSLGAIVSCYGKTKCTASNKNR
GIIKTFSNGCDYVSNKGVDTVSVGNTLYCVNKQEGKSLYVKGEPIINFY
DPLVFPSDEFDASISQVNEKINQSLAFIR(KSDELL)
sc9-10 DS-Cav1 A149C Y458C S46G K465Q S215P E92D
(SEQ ID NO: 62)
(MELLILKANAITTILTAVTFCFASG)QNITEEFYQSTCSAVSKGYLGA
LRTGWYTSVITIELSNIKENKCNGTDAKVKLIKQELDKYKNAVTDLQLL
MQSTPATGSGSAICSGVAVCKVLHLEGEVNKIKSALLSTNKAVVSLSNG
VSVLTFKVLDLKNYIDKQLLPILNKQSCSIPNIETVIEFQQKNNRLLEI
TREFSVNAGVTTPVSTYMLTNSELLSLINDMPITNDQKKLMSNNVQIVR
QQSYSIMCIIKEEVLAYVVQLPLYGVIDTPCWKLHTSPLCTTNTKEGSN
ICLTRTDRGWYCDNAGSVSFFPQAETCKVQSNRVFCDTMNSRTLPSEVN
LCNVDIFNPKYDCKIMTSKTDVSSSVITSLGAIVSCYGKTKCTASNKNR
GIIKTFSNGCDYVSNKGVDTVSVGNTLYCVNKQEGQSLYVKGEPIINFY
DPLVFPSDEFDASISQVNEKINQSLAFIR(KSDELL)

SEQ ID NO:61-62 represent second-generation stabilized DS-Cav1 immunogens; mutations relative to DS-Cav1 are noted and it should be noted that the present disclosure contemplates the use of DS-Cav1 mutants that differ by a single one of the noted amino acid substitutions in SEQ ID NO:61 or 62 above, or two or more of the amino acid substitutions noted. In other embodiments, the F protein may comprise one or more of the following, each of which may additionally include 1, 2, or more of the noted amino acid substitutions in SEQ ID NO:61 or 62 above:

RSV F SC-DM (N67I, S215P)
(SEQ ID NO: 63)
(MELLILKANAITTILTAVTFCFASG)QNITEEFYQSTCSAVSKGYLSALRTGWYTSVITIELSN
IKKIKCNGTDAKIKLIKQELDKYKNAVTELQLLMQSTPATNNQARGSGSGRSLGFLLGVGSAIAS
GVAVSKVLHLEGEVNKIKSALLSTNKAVVSLSNGVSVLTSKVLDLKNYIDKQLLPIVNKQSCSIP
NIETVIEFQQKNNRLLEITREFSVNAGVTTPVSTYMLTNSELLSLINDMPITNDQKKLMSNNVQI
VRQQSYSIMSIIKEEVLAYVVQLPLYGVIDTPCWKLHTSPLCTTNTKEGSNICLTRTDRGWYCDN
AGSVSFFPQAETCKVQSNRVFCDTMNSLTLPSEVNLCNVDIFNPKYDCKIMTSKTDVSSSVITSL
GAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIR(KSDELLSAIGGYIPEAPRDGQAYVRKDGEWVL
LSTFL)
SC-TM (N671, S215P, and E487Q)
(SEQ ID NO: 64)
(MELLILKANAITTILTAVTFCFASG)QNITEEFYQSTCSAVSKGYLSALRTGWYTSVITIELSN
IKKIKCNGTDAKIKLIKQELDKYKNAVTELQLLMQSTPATNNQARGSGSGRSLGFLLGVGSAIAS
GVAVSKVLHLEGEVNKIKSALLSTNKAVVSLSNGVSVLTSKVLDLKNYIDKQLLPIVNKQSCSIP
NIETVIEFQQKNNRLLEITREFSVNAGVTTPVSTYMLTNSELLSLINDMPITNDQKKLMSNNVQI
VRQQSYSIMSIIKEEVLAYVVQLPLYGVIDTPCWKLHTSPLCTTNTKEGSNICLTRTDRGWYCDN
AGSVSFFPQAETCKVQSNRVFCDTMNSLTLPSEVNLCNVDIFNPKYDCKIMTSKTDVSSSVITSL
GAIVSCYGKTKCTASNKNRGIIKTFSNGCDYVSNKGVDTVSVGNTLYYVNKQEGKSLYVKGEPII
NFYDPLVFPSDQFDASISQVNEKINQSLAFIR(KSDELLSAIGGYIPEAPRDGQAYVRKDGEWVL
LSTFL)
HMPV F protein, strain CAN97-83 (A2)
(SEQ ID NO: 65)
(MSWKVVIIFSLLITPQHG)LKESYLEESCSIITEGYLSVLRTGWYTNVFTLEVGDVENLICSDG
PSLIKTELDLTKSALRELKTVSADQLAREEQIENPRQSRFVLGAIALGVATAAAVTAGVAIAKTI
RLESEVTAIKNALKTTNEAVSTLGNGVRVLATAVRELKDFVSKNLTRAINKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTAAGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPIKFPE
DQFNVALDQVFENIENSQALVDQSNRILSSAEKGNIG
HMPVF with A113C, A339C, T160F, I177L
(SEQ ID NO: 66)
(MSWKVVIIFSLLITPQHG)LKESYLEESCSIITEGYLSVLRTGWYTNVFTLEVGDVENLICSDG
PSLIKTELDLTKSALRELKTVSADQLAREEQIENPRQSRFVLGAIALGVCTAAAVTAGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPIKFPE
DQFNVALDQVFENIENSQALVDQSNRILSSAEKGNIG
HMPV F with A113C, A120C, A339C, T160F, I177L, and Q426C
(SEQ ID NO: 67)
(MSWKVVIIFSLLITPQHG)LKESYLEESCSTITEGYLSVLRTGWYTNVFTLEVGDVENLICSDG
PSLIKTELDLTKSALRELKTVSADQLAREEQIENPRQSRFVLGAIALGVCTAAAVTCGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYCLSKVEGEQHVIKGRPVSSSFDPIKFPE
DQFNVALDQVFENIENSQALVDQSNRILSSAEKGNIG
HMPV F_>AAK62968.2 fusion protein [Human metapneumovirus]
(SEQ ID NO: 101)
(MSWKVVIIFSLLITPQHG)LKESYLEESCSTITEGYLSVLRTGWYTNVFTLEVGDVENLICADG
PSLIKTELDLIKSALRELRIVSADQLAREEQIENPRQSRFVLGAIALGVATAAAVTAGVAIAKTI
RLESEVTAIKNALKKTNEAVSTLGNGVRVLATAVRELKDFVSKNLTRAINKNKCDIADLKMAVSF
SQFNRRFLNVVRQFSDNAGITPAISLDLMTDAELARAVSNMPTSAGQIKLMLENRAMVRRKGFGF
LIGVYGSSVIYMVQLPIFGVIDTPCWIVKAAPSCSGKKGNYACLLREDQGWYCQNAGSTVYYPNE
KDCETRGDHVFCDTAAGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPVKFPE
DQFNVALDQVFESIENSQALVDQSNRILSSAEKGNIG
115-BV (A185P)
(SEQ ID NO: 68)
(MSWKVVIIFSLLITPQHG)LKESYLEESCSTITEGYLSVLRTGWYTNVFTLEVGDVENLICADG
PSLIKTELDLIKSALRELRIVSADQLAREEQIENPRRRRFVLGAIALGVATAAAVTAGVAIAKTI
RLESEVTAIKNALKKTNEAVSTLGNGVRVLATAVRELKDFVSKNLTRAINKNKCDIPDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSEKKGNYACLLREDQGWYCQNAGSTVYYPNE
KDCETRGDHVFCDTAAGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPVKFPE
DQFNVALDQVFESIENSQALVDQSNRILSSAEKGNT(SGRENLYFQGGGGSGYIPEAPRDGQAYV
RKDGEWVLLSTFLGGIEGRHHHHHH)

In other embodiments, the one or more paramyxovirus and/or pneumovirus F proteins, or antigenic fragments thereof, may comprise a polypeptide having at least 75%, 80%, 85%, 90%, 91%, 92%, 93%, 94%, 95%, 96%, 97%, 98%, 99%, or 100% identity along its full length to an RSV F protein or mutant thereof selected from the group consisting of SEQ ID NO:53 and 61-64, wherein the polypeptide includes one or more of the following residues: 67I, 149C, 458C, 46G, 465Q, 215P, 92D, and 487Q.

In other embodiments, the one or more paramyxovirus and/or pneumovirus F proteins, or antigenic fragments thereof, may comprise a polypeptide having at least 75%, 80%, 85%, 90%, 91%, 92%, 93%, 94%, 95%, 96%, 97%, 98%, 99%, or 100% identity along its full length to an MPV F protein or mutant thereof selected from the group consisting of SEQ ID NO:65-68 and 101, wherein the polypeptide includes one or more of the following residues: 113C, 120C, 339C, 160F, 177L, 185P, and 426C.

### Linker Between F Proteins and Trimeric Assembly Domains and Geometric Requirements

In the nanostructures of the invention, the F protein and the trimeric assembly domain may be genetically fused such that they are both present in a single polypeptide. Preferably, the linkage between the F protein and the trimeric assembly domain allows the F protein, or antigenic fragment thereof, to be displayed on the exterior of the nanostructures of the invention. As such, the point of connection to the trimeric assembly domain should be on the exterior of the nanostructure formed by the trimeric assembly domain and the second assembly domain in the absence of any F protein. As will be understood by those of skill in the art, a wide variety of polypeptide sequences can be used to link the paramyxovirus and/or pneumovirus F proteins, or antigenic fragments thereof and the trimeric assembly domain. These polypeptide sequences are referred to as linkers. Any suitable linker can be used; there is no amino acid sequence requirement to serve as an appropriate linker. There is no requirement that the linker impose a rigid relative orientation of the F protein or antigenic fragment thereof to the trimeric assembly domain beyond enabling the F protein or antigenic fragment thereof to be displayed on the exterior of the nanostructures of the invention. In some embodiments, the linker includes additional trimerization domains (e.g., the foldon domain of T4 fibritin) that assist in stabilizing the trimeric form of the F protein.

### T4 Fibritin Foldon Domain (Optional in the Linker Region) (SEQ ID NO:54)

### GYIPEAPRDGQAYVRKDGEWVLLSTFL

In other embodiments, the linker may comprise a Gly-Ser linker (i.e.: a linker consisting of glycine and serine residues) of any suitable length. In various embodiments, the Gly-Ser linker may be 3, 4, 5, 6, 7, 8, 9, 10, 11, 12, 13, 14, 15, 16, 17, 18, 19, 20, or more amino acids in length. In various embodiments, the Gly-Ser linker may comprise or consist of the amino acid sequence of GSGGSGSGSGGSGSG (SEQ ID NO:55), GGSGGSGS (SEQ ID NO:56) or GSGGSGSG (SEQ ID NO:57).

In further embodiments the linker may comprise a helical extension domain that may serve to extend the N-terminal helix of the first polypeptide, when expressed as a fusion polypeptide with the one or more paramyxovirus and/or pneumovirus F proteins, or antigenic fragments thereof, so that it is located at the exterior of the nanostructure surface. The helical extension may be present in combination with the other linker components described herein, or may be absent. The helical extension may be of any suitable length (i.e.: 7, 8, 9, 10, 11, 12, or more amino acids) and comprise any suitable primary amino acid sequence. In one embodiment, the helical extension may comprise or consist of the amino acid sequence EKAAKAEEAAR (SEQ ID NO:58).

Thus, in various non-limiting embodiments in which the F protein is present as a fusion protein with the first polypeptide and a linker is used, the F protein-linker sequence may comprise the following (exemplified by DS-Cav1 as the F protein in these non-limiting embodiments). Residues in parentheses are optional and the amino acid sequence MELLILKANAITTILTAVTFCFASG (SEQ ID NO:59) represents the N-terminal DS-Cav1 signal peptide that is cleaved during processing:

DS-Cav1-foldon (SEQ ID NO: 60):
(MELLILKANAITTILTAVTFCFASG)QNITEEFYQSTCSAVSKGYLSA
LRTGWYTSVITIELSNIKENKCNGTDAKVKLIKQELDKYKNAVTELQLL
MQSTPATNNRARRELPRFMNYTLNNAKKTNVTLSKKRKRRFLGFLLGVG
SAIASGVAVCKVLHLEGEVNKIKSALLSTNKAVVSLSNGVSVLTFKVLD
LKNYIDKQLLPILNKQSCSISNIETVIEFQQKNNRLLEITREFSVNAGV
TTPVSTYMLTNSELLSLINDMPITNDQKKLMSNNVQIVRQQSYSIMCII
KEEVLAYVVQLPLYGVIDTPCWKLHTSPLCTINTKEGSNICLTRTDRGW
YCDNAGSVSFFPQAETCKVQSNRVFCDTMNSLTLPSEVNLCNVDIFNPK
YDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEF
DASISQVNEKINQSLAFIRKSDELLGYIPEAPRDGQAYVRKDGEWVLLS
TFL

In various further embodiments, the first polypeptides comprise or consist of fusion polypeptides of first polypeptides fused to an F protein, where the fusion protein has a sequence selected from the following (optional residues in parentheses):

DS-Cav1-foldon-T33-31A
(SEQ ID NO: 69)
(MELLILKANVIATILTAVTFCFASS)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRKSDELLGYIPE
APRDGQAYVRKDGEWVLLSTFLGGSMEEVVLITVPSALVAVKIAHALVEERLAACVNIVPGLTSI
YREEGSVVSDHELLLLVKITTDAFPKLKERVKELHPYEVPEIVALPIAEGNREYLDWLRENTG
DS-Cav1-T33-31A
(SEQ ID NO: 70)
(MELLILKANVIATILTAVTFCFASS)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRKSDELLGGSME
EVVLITVPSALVAVKIAHALVEERLAACVNIVPGLTSIYREEGSVVSDHELLLLVKITTDAFPKL
KERVKELHPYEVPEIVALPIAEGNREYLDWLRENTG
DS-Cav1-foldon-T33-15B
(SEQ ID NO: 71)
(MELLILKANVIATILTAVTFCFASS)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRKSDELLGYIPE
APRDGQAYVRKDGEWVLLSTFLGGSMVRGIRGAITVNSDTPTSIIIATILLLEKMLEANGIQSYE
ELAAVIFTVTEDLTSAFPAEAARQIGMHRVPLLSAREVPVPGSLPRVIRVLALWNTDTPQDRVRH
VYLSEAVRLRPDLESAQ
DS-Cav1-T33-15B
(SEQ ID NO: 72)
(MELLILKANVIATILTAVTFCFASS)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRKSDELLGGSMV
RGIRGAITVNSDTPTSIIIATILLLEKMLEANGIQSYEELAAVIFTVTEDLTSAFPAEAARQIGM
HRVPLLSAREVPVPGSLPRVIRVLALWNTDTPQDRVRHVYLSEAVRLRPDLESAQ
DS-Cav1-foldon-I53-50A
(SEQ ID NO: 73)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGYIPEAPRDGQ
AYVRKDGEWVLLSTFLGSGSHHHHHHHHGGSGGSGSEKAAKAEEAARKMEELFKKHKIVAVLRAN
SVEEAIEKAVAVFAGGVHLIEITFTVPDADTVIKALSVLKEKGAIIGAGTVTSVEQCRKAVESGA
EFIVSPHLDEEISQFCKEKGVFYMPGVMTPTELVKAMKLGHTILKLFPGEVVGPQFVKAMKGPFP
NVKFVPTGGVNLDNVCEWFKAGVLAVGVGSALVKGTPDEVREKAKAFVEKIRGCTE
DS-Cav1-I53-50A
(SEQ ID NO: 74)
(MELLILKANVIATILTAVTFCFASS)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGGSGGSGSEKA
AKAEEAARKMEELFKKHKIVAVLRANSVEEAIEKAVAVFAGGVHLIEITFTVPDADTVIKALSVL
KEKGAIIGAGTVTSVEQCRKAVESGAEFIVSPHLDEEISQFCKEKGVFYMPGVMTPTELVKAMKL
GHTILKLFPGEVVGPQFVKAMKGPFPNVKFVPTGGVNLDNVCEWFKAGVLAVGVGSALVKGTPDE
VREKAKAFVEKIRGCTE
DS-Cav1-I32-28A
(SEQ ID NO: 75)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRKSDELLGGSGG
SGSDDARIAAIGDVDELNSQIGVLLAEPLPDDVRAALSAIQHDLFDLGGELCIPGHAAITEDHLL
RLALWLVHYNGQLPPLEEFILPGGARGAALAHVCRTVCRRAERSIKALGASEPLNIAPAAYVNLL
SDLLFVLARVLNRAAGGADVLWDRTRAH
DS-Cav1-8GS-HelExt-I53-50A (F10)
(SEQ ID NO: 76)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGSGGSGSGEKA
AKAEEAARKMEELFKKHKIVAVLRANSVEEAIEKAVAVFAGGVHLIEITFTVPDADTVIKALSVL
KEKGAIIGAGTVTSVEQCRKAVESGAEFIVSPHLDEEISQFCKEKGVFYMPGVMTPTELVKAMKL
GHTILKLFPGEVVGPQFVKAMKGPFPNVKFVPTGGVNLDNVCEWFKAGVLAVGVGSALVKGTPDE
VREKAKAFVEKIRGCTE
DS-Cav1-foldon-15GS-HelExt-I53-50A (F14)
(SEQ ID NO: 77)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGYIPEAPRDGQ
AYVRKDGEWVLLSTFLGSGGSGSGSGGSGSGEKAAKAEEAARKMEELFKKHKIVAVLRANSVEEA
IEKAVAVFAGGVHLIEITFTVPDADTVIKALSVLKEKGAIIGAGTVTSVEQCRKAVESGAEFIVS
PHLDEEISQFCKEKGVFYMPGVMTPTELVKAMKLGHTILKLFPGEVVGPQFVKAMKGPFPNVKFV
PTGGVNLDNVCEWFKAGVLAVGVGSALVKGTPDEVREKAKAFVEKIRGCTE
HMPV F wt_CAN97-83 strain-I53-50A
(SEQ ID NO: 78)
(MSWKVVIIFSLLITPQHG)LKESYLEESCSTITEGYLSVLRTGWYINVFTLEVGDVENLICSDG
PSLIKTELDLTKSALRELKTVSADQLAREEQIENPRQSRFVLGAIALGVATAAAVTAGVAIAKTI
RLESEVTAIKNALKTTNEAVSTLGNGVRVLATAVRELKDFVSKNLTRAINKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTAAGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPIKFPE
DQFNVALDQVFENIENSQALVDQSNRILSSAEKGNTGFIIVIILIAVLGSSMILVSIFIIIKKTK
KPTGAPPELSGVTNNGFIPHSGSGSHHHHHHHHGGSGGSGSEKAAKAEEAARKMEELFKKHKIVA
VLRANSVEEAIEKAVAVFAGGVHLIEITFTVPDADTVIKALSVLKEKGAIIGAGTVTSVEQCRKA
VESGAEFIVSPHLDEEISQFCKEKGVFYMPGVMTPTELVKAMKLGHTILKLFPGEVVGPQFVKAM
KGPFPNVKFVPTGGVNLDNVCEWFKAGVLAVGVGSALVKGTPDEVREKAKAFVEKIRGCTE
HMPV F A113C_A339C_T160F_I177L-I53-50A
(SEQ ID NO: 79)
(MSWKVVIIFSLLITPQHG)LKESYLEESCSTITEGYLSVLRTGWYINVFTLEVGDVENLICSDG
PSLIKTELDLTKSALRELKTVSADQLAREEQIENPRQSRFVLGAIALGVCTAAAVTAGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPIKFPE
DQFNVALDQVFENIENSQALVDQSNRILSSAEKGNTGGSGSHHHHHHHHGGSGGSGSEKAAKAEE
AARKMEELFKKHKIVAVLRANSVEEAIEKAVAVFAGGVHLIEITFTVPDADTVIKALSVLKEKGA
IIGAGTVTSVEQCRKAVESGAEFIVSPHLDEEISQFCKEKGVFYMPGVMTPTELVKAMKLGHTIL
KLFPGEVVGPQFVKAMKGPFPNVKFVPTGGVNLDNVCEWFKAGVLAVGVGSALVKGTPDEVREKA
KAFVEKIRGCTE
HMPV F A113C_A339C_T160F_I177L_A120C, Q426C mutations-I53-50A
(SEQ ID NO: 80)
(MSWKVVIIFSLLITPQHG)LKESYLEESCSTITEGYLSVLRTGWYINVFTLEVGDVENLICSDG
PSLIKTELDLTKSALRELKTVSADQLAREEQIENPRQSRFVLGAIALGVCTAAAVTCGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYCLSKVEGEQHVIKGRPVSSSFDPIKFPE
DQFNVALDQVFENIENSQALVDQSNRILSSAEKGNTGGSGSHHHHHHHHGGSGGSGSEKAAKAEE
AARKMEELFKKHKIVAVLRANSVEEAIEKAVAVFAGGVHLIEITFTVPDADTVIKALSVLKEKGA
IIGAGTVTSVEQCRKAVESGAEFIVSPHLDEEISQFCKEKGVFYMPGVMTPTELVKAMKLGHTIL
KLFPGEVVGPQFVKAMKGPFPNVKFVPTGGVNLDNVCEWFKAGVLAVGVGSALVKGTPDEVREKA
KAFVEKIRGCTE
sc-DS2-I53-50A
(SEQ ID NO: 81)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATGSGSCIASGVAVCKVLHLEGEVNKI
KSALLSTNKAVVSLSNGVSVLTFKVLDLKNYIDKQLLPILNKQSCSISNIETVIEFQQKNNRLLE
ITREFSVNAGVTTPVSTYMLINSELLSLINDMPITNDQKKLMSNNVQIVRQQSYSIMCIIKEEVL
AYVVQLPLYGVIDTPCWKLHTSPLCTTNTKEGSNICLTRTDRGWYCDNAGSVSFFPQAETCKVQS
NRVFCDTMNSLTLPSEVNLCNVDIFNPKYDCKIMTSKTDVSSSVITSLGAIVSCYGKTKCTASNK
NRGIIKTFSNGCDYVSNKGVDTVSVGNTLYCVNKQEGKSLYVKGEPIINFYDPLVFPSDEFDASI
SQVNEKINQSLAFIRGSGSHHHHHHHHGGSGGSGSEKAAKAEEAARKMEELFKKHKIVAVLRANS
VEEAIEKAVAVFAGGVHLIEITFTVPDADTVIKALSVLKEKGAIIGAGTVTSVEQCRKAVESGAE
FIVSPHLDEEISQFCKEKGVFYMPGVMTPTELVKAMKLGHTILKLFPGEVVGPQFVKAMKGPFPN
VKFVPTGGVNLDNVCEWFKAGVLAVGVGSALVKGTPDEVREKAKAFVEKIRGCTE
tc-DS2-I53-50A
(SEQ ID NO: 82)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSCIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYCVNKQEGKSLYVKGEPIINFYDPLVFPSDEFDASISQVNEKINQSLAFIRGSGSHHHHHHH
HGGSGGSGSEKAAKAEEAARKMEELFKKHKIVAVLRANSVEEAIEKAVAVFAGGVHLISITFTVP
DADTVIKALSVLKEKGAIIGAGTVTSVEQCRKAVESGAEFIVSPHLDEEISQFCKEKGVFYMPGV
MTPTELVKAMKLGHTILKLFPGEVVGPQFVKAMKGPFPNVKFVPTGGVNLDNVCEWFKAGVLAVG
VGSALVKGTPDEVREKAKAFVEKIRGCTE
DS-Cav1-12GS-HelExt-I53-50A (F11)
(SEQ ID NO: 83)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGSGGSGSGSGG
SEKAAKAEEAARKMEELFKKHKIVAVLRANSVEEAIEKAVAVFAGGVHLIEliFTVPDADTVIKA
LSVLKEKGAIIGAGTVTSVEQCRKAVESGAEFIVSPHLDEEISQFCKEKGVFYMPGVMTPTELVK
AMKLGHTILKLFPGEVVGPQFVKAMKGPFPNVKFVPTGGVNLDNVCEWFKAGVLAVGVGSALVKG
TPDEVREKAKAFVEKIRGCTE
DS-Cav1-16GS-HelExt-I53-50A (F12)
(SEQ ID NO: 84)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGSGGSGSGSGG
SGSGGEKAAKAEEAARKMEELFKKHKIVAVLRANSVEEAIEKAVAVFAGGVHLIEliFTVPDADT
VIKALSVLKEKGAIIGAGTVTSVEQCRKAVESGAEFIVSPHLDEEISQFCKEKGVFYMPGVMTPT
ELVKAMKLGHTILKLFPGEVVGPQFVKAMKGPFPNVKFVPTGGVNLDNVCEWFKAGVLAVGVGSA
LVKGTPDEVREKAKAFVEKIRGCTE
DS-Cav1-foldon-10GS-HelExt-I53-50A (F13
(SEQ ID NO: 85)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGYIPEAPRDGQ
AYVRKDGEWVLLSTFLGSGGSGSGSGEKAAKAEEAARKMEELFKKHKIVAVLRANSVEEAIEKAV
AVFAGGVHLIEITFTVPDADTVIKALSVLKEKGAIIGAGTVTSVEQCRKAVESGAEFIVSPHLDE
EISQFCKEKGVFYMPGVMTPTELVKAMKLGHTILKLFPGEVVGPQFVKAMKGPFPNVKFVPTGGV
NLDNVCEWFKAGVLAVGVGSALVKGTPDEVREKAKAFVEKIRGCTE
DS-Cav1-foldon-20GS-HelExt-I53-50A (F15)
(SEQ ID NO: 86)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATNNRARRELPRFMNYTLNNAKKTNVT
LSKKRKRRFLGFLLGVGSAIASGVAVCKVLHLEGEVNKIKSALLSTNKAVVSLSNGVSVLTFKVL
DLKNYIDKQLLPILNKQSCSISNIETVIEFQQKNNRLLEITREFSVNAGVTTPVSTYMLTNSELL
SLINDMPITNDQKKLMSNNVQIVRQQSYSIMCIIKEEVLAYVVQLPLYGVIDTPCWKLHTSPLCT
INTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVG
NTLYYVNKQEGKSLYVKGEPIINFYDPLVFPSDEFDASISQVNEKINQSLAFIRGYIPEAPRDGQ
AYVRKDGEWVLLSTFLGSGGSGSGSGGSGSGGSSGSEKAAKAEEAARKMEELFKKHKIVAVLRAN
SVEEAIEKAVAVFAGGVHLIEITFTVPDADTVIKALSVLKEKGAIIGAGTVTSVEQCRKAVESGA
EFIVSPHLDEEISQFCKEKGVFYMPGVMTPTELVKAMKLGHTILKLFPGEVVGPQFVKAMKGPFP
NVKFVPTGGVNLDNVCEWFKAGVLAVGVGSALVKGTPDEVREKAKAFVEKIRGCTE
sc9-10 DS-Cav1 A149C Y458C-foldon-I53-50A embodiment
(SEQ ID NO: 87)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATGSGSAICSGVAVCKVLHLEGEVNKI
KSALLSTNKAVVSLSNGVSVLTFKVLDLKNYIDKQLLPILNKQSCSISNIETVIEFQQKNNRLLE
ITREFSVNAGVTTPVSTYMLINSELLSLINDMPITNDQKKLMSNNVQIVRQQSYSIMCIIKEEVL
AYVVQLPLYGVIDTPCWKLHTSPLCTINTKEGSNICLTRTDRGWYCDNAGSVSFFPQAETCKVQS
NRVFCDTMNSRTLPSEVNLCNVDIFNPKYDCKIMTSKTDVSSSVITSLGAIVSCYGKTKCTASNK
NRGIIKTFSNGCDYVSNKGVDTVSVGNTLYCVNKQEGKSLYVKGEPIINFYDPLVFPSDEFDASI
SQVNEKINQSLAFIR(KSDELL)GYIPEAPRDGQAYVRKDGEWVLLSTFLGSGSHHHHHHHHGGS
GGSGSEKAAKAEEAARKMEELFKKHKIVAVLRANSVEEAIEKAVAVFAGGVHLIEITFTVPDADT
VIKALSVLKEKGAIIGAGTVTSVEQCRKAVESGAEFIVSPHLDEEISQFCKEKGVFYMPGVMTPT
ELVKAMKLGHTILKLFPGEVVGPQFVKAMKGPFPNVKFVPTGGVNLDNVCEWFKAGVLAVGVGSA
LVKGTPDEVREKAKAFVEKIRGCTE
sc9-10 DS-Cav1 A149C Y458C-I53-50A - F10 embodiment
(SEQ ID NO: 88)
(MELLILKANAITTILTAVTFCFASG)QNITEEFYQSTCSAVSKGYLSALRTGWYTSVITIELSN
IKENKCNGTDAKVKLIKQELDKYKNAVTELQLLMQSTPATGSGSAICSGVAVCKVLHLEGEVNKI
KSALLSTNKAVVSLSNGVSVLTFKVLDLKNYIDKQLLPILNKQSCSISNIETVIEFQQKNNRLLE
ITREFSVNAGVTTPVSTYMLINSELLSLINDMPITNDQKKLMSNNVQIVRQQSYSIMCIIKEEVL
AYVVQLPLYGVIDTPCWKLHTSPLCTINTKEGSNICLTRTDRGWYCDNAGSVSFFPQAETCKVQS
NRVFCDTMNSRTLPSEVNLCNVDIFNPKYDCKIMTSKTDVSSSVITSLGAIVSCYGKTKCTASNK
NRGIIKTFSNGCDYVSNKGVDTVSVGNTLYCVNKQEGKSLYVKGEPIINFYDPLVFPSDEFDASI
SQVNEKINQSLAFIR(KSDELL)GSGGSGSGEKAAKAEEAARKMEELFKKHKIVAVLRANSVEEA
IEKAVAVFAGGVHLIEITFTVPDADTVIKALSVLKEKGAIIGAGTVTSVEQCRKAVESGAEFIVS
PHLDEEISQFCKEKGVFYMPGVMTPTELVKAMKLGHTILKLFPGEVVGPQFVKAMKGPFPNVKFV
PTGGVNLDNVCEWFKAGVLAVGVGSALVKGTPDEVREKAKAFVEKIRGCTE
sc9-10 DS-Cav1 A149C Y458C S46G K465Q S215P E92D-foldon-I53-50A
embodiment
(SEQ ID NO: 89)
(MELLILKANAITTILTAVTFCFASG)QNITEEFYQSTCSAVSKGYLGALRTGWYTSVITIELSN
IKENKCNGTDAKVKLIKQELDKYKNAVTDLQLLMQSTPATGSGSAICSGVAVCKVLHLEGEVNKI
KSALLSTNKAVVSLSNGVSVLTFKVLDLKNYIDKQLLPILNKQSCSIPNIETVIEFQQKNNRLLE
ITREFSVNAGVTTPVSTYMLINSELLSLINDMPITNDQKKLMSNNVQIVRQQSYSIMCIIKEEVL
AYVVQLPLYGVIDTPCWKLHTSPLCTTNTKEGSNICLTRTDRGWYCDNAGSVSFFPQAETCKVQS
NRVFCDTMNSRTLPSEVNLCNVDIFNPKYDCKIMTSKTDVSSSVITSLGAIVSCYGKTKCTASNK
NRGIIKTFSNGCDYVSNKGVDTVSVGNTLYCVNKQEGQSLYVKGEPIINFYDPLVFPSDEFDASI
SQVNEKINQSLAFIR(KSDELL)GYIPEAPRDGQAYVRKDGEWVLLSTFLGSGSHHHHHHHHGGS
GGSGSEKAAKAEEAARKMEELFKKHKIVAVLRANSVEEAIEKAVAVFAGGVHLIEliFTVPDADT
VIKALSVLKEKGAIIGAGTVTSVEQCRKAVESGAEFIVSPHLDEEISQFCKEKGVFYMPGVMTPT
ELVKAMKLGHTILKLFPGEVVGPQFVKAMKGPFPNVKFVPTGGVNLDNVCEWFKAGVLAVGVGSA
LVKGTPDEVREKAKAFVEKIRGCTE
sc9-10 DS-Cav1 A149C Y458C S46G K465Q S215P E92D-I53-50A - F10
embodiment
(SEQ ID NO: 90)
(MELLILKANAITTILTAVTFCFASG)QNITEEFYQSTCSAVSKGYLGALRTGWYTSVITIELSN
IKENKCNGTDAKVKLIKQELDKYKNAVTDLQLLMQSTPATGSGSAICSGVAVCKVLHLEGEVNKI
KSALLSTNKAVVSLSNGVSVLTFKVLDLKNYIDKQLLPILNKQSCSIPNIETVIEFQQKNNRLLE
ITREFSVNAGVTTPVSTYMLINSELLSLINDMPITNDQKKLMSNNVQIVRQQSYSIMCIIKEEVL
AYVVQLPLYGVIDTPCWKLHTSPLCTTNTKEGSNICLTRTDRGWYCDNAGSVSFFPQAETCKVQS
NRVFCDTMNSRTLPSEVNLCNVDIFNPKYDCKIMTSKTDVSSSVITSLGAIVSCYGKTKCTASNK
NRGIIKTFSNGCDYVSNKGVDTVSVGNTLYCVNKQEGQSLYVKGEPIINFYDPLVFPSDEFDASI
SQVNEKINQSLAFIR(KSDELL)GSGGSGSGEKAAKAEEAARKMEELFKKHKIVAVLRANSVEEA
IEKAVAVFAGGVHLIEITFTVPDADTVIKALSVLKEKGAIIGAGTVTSVEQCRKAVESGAEFIVS
PHLDEEISQFCKEKGVFYMPGVMTPTELVKAMKLGHTILKLFPGEVVGPQFVKAMKGPFPNVKFV
PTGGVNLDNVCEWFKAGVLAVGVGSALVKGTPDEVREKAKAFVEKIRGCTE
SC-DM (N67I, S215P) - foldon-I53-50A embodiment
(SEQ ID NO: 91)
(MELLILKANAITTILTAVTFCFASG)QNITEEFYQSTCSAVSKGYLSALRTGWYTSVITIELSN
IKKIKCNGTDAKIKLIKQELDKYKNAVTELQLLMQSTPATNNQARGSGSGRSLGFLLGVGSAIAS
GVAVSKVLHLEGEVNKIKSALLSTNKAVVSLSNGVSVLTSKVLDLKNYIDKQLLPIVNKQSCSIP
NIETVIEFQQKNNRLLEITREFSVNAGVTTPVSTYMLTNSELLSLINDMPITNDQKKLMSNNVQI
VRQQSYSIMSIIKEEVLAYVVQLPLYGVIDTPCWKLHTSPLCTTNTKEGSNICLTRTDRGWYCDN
AGSVSFFPQAETCKVQSNRVFCDTMNSLTLPSEVNLCNVDIFNPKYDCKIMTSKTDVSSSVITSL
GAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIR(KSDELL)GYIPEAPRDGQAYVRKDGEWVLLST
FLGSGSHHHHHHHHGGSGGSGSEKAAKAEEAARKMEELFKKHKIVAVLRANSVEEAIEKAVAVFA
GGVHLIEITFTVPDADTVIKALSVLKEKGAIIGAGTVTSVEQCRKAVESGAEFIVSPHLDEEISQ
FCKEKGVFYMPGVMTPTELVKAMKLGHTILKLFPGEVVGPQFVKAMKGPFPNVKFVPTGGVNLDN
VCEWFKAGVLAVGVGSALVKGTPDEVREKAKAFVEKIRGCTE
SC-DM (N67I, S215P)-I53-50A - F10 embodiment
(SEQ ID NO: 92)
(MELLILKANAITTILTAVTFCFASG)QNITEEFYQSTCSAVSKGYLSALRTGWYTSVITIELSN
IKKIKCNGTDAKIKLIKQELDKYKNAVTELQLLMQSTPATNNQARGSGSGRSLGFLLGVGSAIAS
GVAVSKVLHLEGEVNKIKSALLSTNKAVVSLSNGVSVLTSKVLDLKNYIDKQLLPIVNKQSCSIP
NIETVIEFQQKNNRLLEITREFSVNAGVTTPVSTYMLTNSELLSLINDMPITNDQKKLMSNNVQI
VRQQSYSIMSIIKEEVLAYVVQLPLYGVIDTPCWKLHTSPLCTTNTKEGSNICLTRTDRGWYCDN
AGSVSFFPQAETCKVQSNRVFCDTMNSLTLPSEVNLCNVDIFNPKYDCKIMTSKTDVSSSVITSL
GAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIR(KSDELL)GSGGSGSGEKAAKAEEAARKMEELF
KKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMKLGHTILKLFPGEVVG
PQFVKAMKGPFPNVKFVPTGGVNLDNVCEWFKAGVLAVGVGSALVKGTPDEVREKAKAFVEKIRG
CTE
SC-TM (N67I, S215P, and E487Q) - foldon-I53-50A embodiment
(SEQ ID NO: 93)
(MELLILKANAITTILTAVTFCFASG)QNITEEFYQSTCSAVSKGYLSALRTGWYTSVITIELSN
IKKIKCNGTDAKIKLIKQELDKYKNAVTELQLLMQSTPATNNQARGSGSGRSLGFLLGVGSAIAS
GVAVSKVLHLEGEVNKIKSALLSTNKAVVSLSNGVSVLTSKVLDLKNYIDKQLLPIVNKQSCSIP
NIETVIEFQQKNNRLLEITREFSVNAGVTTPVSTYMLTNSELLSLINDMPITNDQKKLMSNNVQI
VRQQSYSIMSIIKEEVLAYVVQLPLYGVIDTPCWKLHTSPLCTTNTKEGSNICLTRTDRGWYCDN
AGSVSFFPQAETCKVQSNRVFCDTMNSLTLPSEVNLCNVDIFNPKYDCKIMTSKTDVSSSVITSL
GAIVSCYGKTKCTASNKNRGIIKTFSNGCDYVSNKGVDTVSVGNTLYYVNKQEGKSLYVKGEPII
NFYDPLVFPSDQFDASISQVNEKINQSLAFIR(KSDELL)GYIPEAPRDGQAYVRKDGEWVLLST
FLGSGSHHHHHHHHGGSGGSGSEKAAKAEEAARKMEELFKKHKIVAVLRANSVEEAIEKAVAVFA
GGVHLIEITFTVPDADTVIKALSVLKEKGAIIGAGTVTSVEQCRKAVESGAEFIVSPHLDEEISQ
FCKEKGVFYMPGVMTPTELVKAMKLGHTILKLFPGEVVGPQFVKAMKGPFPNVKFVPTGGVNLDN
VCEWFKAGVLAVGVGSALVKGTPDEVREKAKAFVEKIRGCTE
SC-TM (N67I, S215P, and E487Q)-I53-50A - F10 embodiment
(SEQ ID NO: 94)
(MELLILKANAITTILTAVTFCFASG)QNITEEFYQSTCSAVSKGYLSALRTGWYTSVITIELSN
IKKIKCNGTDAKIKLIKQELDKYKNAVTELQLLMQSTPATNNQARGSGSGRSLGFLLGVGSAIAS
GVAVSKVLHLEGEVNKIKSALLSTNKAVVSLSNGVSVLTSKVLDLKNYIDKQLLPIVNKQSCSIP
NIETVIEFQQKNNRLLEITREFSVNAGVTTPVSTYMLTNSELLSLINDMPITNDQKKLMSNNVQI
VRQQSYSIMSIIKEEVLAYVVQLPLYGVIDTPCWKLHTSPLCTTNTKEGSNICLTRTDRGWYCDN
AGSVSFFPQAETCKVQSNRVFCDTMNSLTLPSEVNLCNVDIFNPKYDCKIMTSKTDVSSSVITSL
GAIVSCYGKTKCTASNKNRGIIKTFSNGCDYVSNKGVDTVSVGNTLYYVNKQEGKSLYVKGEPII
NFYDPLVFPSDQFDASISQVNEKINQSLAFIR(KSDELL)GSGGSGSGEKAAKAEEAARKMEELF
KKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMKLGHTILKLFPGEVVG
PQFVKAMKGPFPNVKFVPTGGVNLDNVCEWFKAGVLAVGVGSALVKGTPDEVREKAKAFVEKIRG
CTE
HMPV-F with A113C, A339C, T160F, I177L - foldon-I53-50A embodiment
(SEQ ID NO: 95)
(MSWKVVIIFSLLITPQHG)LKESYLEESCSTITEGYLSVLRTGWYTNVFTLEVGDVENLICSDG
PSLIKTELDLTKSALRELKTVSADQLAREEQIENPRQSRFVLGAIALGVCTAAAVTAGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPIKFPE
DQFNVALDQVFENIENSQALVDQSNRILSSAEKGNTGGYIPEAPRDGQAYVRKDGEWVLLSTFLG
SGSHHHHHHHHGGSGGSGSEKAAKAEEAARKMEELFKKHKIVAVLRANSVEEAIEKAVAVFAGGV
HLIEITFTVPDADTVIKALSVLKEKGAIIGAGTVTSVEQCRKAVESGAEFIVSPHLDEEISQFCK
EKGVFYMPGVMTPTELVKAMKLGHTILKLFPGEVVGPQFVKAMKGPFPNVKFVPTGGVNLDNVCE
WFKAGVLAVGVGSALVKGTPDEVREKAKAFVEKIRGCTE
HMPV-F with A113C, A339C, T160F, I177L-I53-50A F10 embodiment
(SEQ ID NO: 96)
(MSWKVVIIFSLLITPQHG)LKESYLEESCSTITEGYLSVLRTGWYTNVFTLEVGDVENLICSDG
PSLIKTELDLTKSALRELKTVSADQLAREEQIENPRQSRFVLGAIALGVCTAAAVTAGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPIKFPE
DQFNVALDQVFENIENSQALVDQSNRILSSAEKGNTGGSGGSGSGEKAAKAEEAARKMEELFKKH
KIVAVLRANSVEEAIEKAVAVFAGGVHLIEITFTVPDADTVIKALSVLKEKGAIIGAGTVTSVEQ
CRKAVESGAEFIVSPHLDEEISQFCKEKGVFYMPGVMTPTELVKAMKLGHTILKLFPGEVVGPQF
VKAMKGPFPNVKFVPTGGVNLDNVCEWFKAGVLAVGVGSALVKGTPDEVREKAKAFVEKIRGCTE
HMPV-F with A113C, A120C, A339C, T160F, I177L, and Q426C - foldon-I53-50A
embodiment
(SEQ ID NO: 97)
(MSWKVVIIFSLLITPQHG)LKESYLEESCSTITEGYLSVLRTGWYINVFTLEVGDVENLICSDG
PSLIKTELDLTKSALRELKTVSADQLAREEQIENPRQSRFVLGAIALGVCTAAAVTCGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYCLSKVEGEQHVIKGRPVSSSFDPIKFPE
DQFNVALDQVFENIENSQALVDQSNRILSSAEKGNTGGYIPEAPRDGQAYVRKDGEWVLLSTFLG
SGSHHHHHHHHGGSGGSGSEKAAKAEEAARKMEELFKKHKIVAVLRANSVEEAIEKAVAVFAGGV
HLIEITFTVPDADTVIKALSVLKEKGAIIGAGTVTSVEQCRKAVESGAEFIVSPHLDEEISQFCK
EKGVFYMPGVMTPTELVKAMKLGHTILKLFPGEVVGPQFVKAMKGPFPNVKFVPTGGVNLDNVCE
WFKAGVLAVGVGSALVKGTPDEVREKAKAFVEKIRGCTE
HMPV-F with A113C, A120C, A339C, T160F, I177L, and Q426C - F10 embodiment
(SEQ ID NO: 98)
(MSWKVVIIFSLLITPQHG)LKESYLEESCSTITEGYLSVLRTGWYINVFTLEVGDVENLICSDG
PSLIKTELDLTKSALRELKTVSADQLAREEQIENPRQSRFVLGAIALGVCTAAAVTCGVAIAKTI
RLESEVTAIKNALKTTNEAVSTLGNGVRVLAFAVRELKDFVSKNLTRALNKNKCDIDDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSGKKGNYACLLREDQGWYCQNAGSTVYYPNE
KDCETRGDHVFCDTACGINVAEQSKECNINISTTNYPCKVSTGRHPISMVALSPLGALVACYKGV
SCSIGSNRVGIIKQLNKGCSYITNQDADTVTIDNTVYCLSKVEGEQHVIKGRPVSSSFDPIKFPE
DQFNVALDQVFENIENSQALVDQSNRILSSAEKGNTGGSGGSGSGEKAAKAEEAARKMEELFKKH
KIVAVLRANSVEEAIEKAVAVFAGGVHLIEITFTVPDADTVIKALSVLKEKGAIIGAGTVTSVEQ
CRKAVESGAEFIVSPHLDEEISQFCKEKGVFYMPGVMTPTELVKAMKLGHTILKLFPGEVVGPQF
VKAMKGPFPNVKFVPTGGVNLDNVCEWFKAGVLAVGVGSALVKGTPDEVREKAKAFVEKIRGCTE
HMPV-F 115-BV (A185P)-foldon-I53-50A embodiment
(SEQ ID NO: 99)
(MSWKVVIIFSLLITPQHG)LKESYLEESCSTITEGYLSVLRTGWYINVFTLEVGDVENLICADG
PSLIKTELDLIKSALRELRIVSADQLAREEQIENPRRRRFVLGAIALGVATAAAVTAGVAIAKTI
RLESEVTAIKNALKKTNEAVSTLGNGVRVLATAVRELKDFVSKNLTRAINKNKCDIPDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSEKKGNYACLLREDQGWYCQNAGSTVYYPNE
KDCETRGDHVFCDTAAGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPVKFPE
DQFNVALDQVFESIENSQALVDQSNRILSSAEKGNTGYIPEAPRDGQAYVRKDGEWVLLSTFLGS
GSHHHHHHHHGGSGGSGSEKAAKAEEAARKMEELFKKHKIVAVLRANSVEEAIEKAVAVFAGGVH
LIEITFTVPDADTVIKALSVLKEKGAIIGAGTVTSVEQCRKAVESGAEFIVSPHLDEEISQFCKE
KGVFYMPGVMTPTELVKAMKLGHTILKLFPGEVVGPQFVKAMKGPFPNVKFVPTGGVNLDNVCEW
FKAGVLAVGVGSALVKGTPDEVREKAKAFVEKIRGCTE
HMPV-F 115-BV (A185P)-I53-50A - F10 embodiment
(SEQ ID NO: 100)
(MSWKVVIIFSLLITPQHG)LKESYLEESCSTITEGYLSVLRTGWYINVFTLEVGDVENLICADG
PSLIKTELDLIKSALRELRIVSADQLAREEQIENPRRRRFVLGAIALGVATAAAVTAGVAIAKTI
RLESEVTAIKNALKKTNEAVSTLGNGVRVLATAVRELKDFVSKNLTRAINKNKCDIPDLKMAVSF
SQFNRRFLNVVRQFSDNAGITPAISLDLMTDAELARAVSNMPTSAGQIKLMLENRAMVRRKGFGI
LIGVYGSSVIYMVQLPIFGVIDTPCWIVKAAPSCSEKKGNYACLLREDQGWYCQNAGSTVYYPNE
KDCETRGDHVFCDTAAGINVAEQSKECNINISTTNYPCKVSTGRHPISMVALSPLGALVACYKGV
SCSIGSNRVGIIKQLNKGCSYITNQDADTVTIDNTVYQLSKVEGEQHVIKGRPVSSSFDPVKFPE
DQFNVALDQVFESIENSQALVDQSNRILSSAEKGNTGSGGSGSGEKAAKAEEAARKMEELFKKHK
IVAVLRANSVEEAIEKAVAVFAGGVHLIEITFTVPDADTVIKALSVLKEKGAIIGAGTVTSVEQC
RKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGCTE

### Second Assemblies

The nanostructures of the invention may comprise multiple copies of a trimeric first assembly and multiple copies of a second assembly. The second assembly comprises a protein-protein interface that induces multiple copies of the second polypeptide to self-associate to form the second assemblies. Multiple oligomeric states of the second assembly may be compatible with nanostructure formation, including dimeric (two copies), trimeric (three copies), tetrameric (four copies), pentameric (five copies), hexameric (six copies), or higher oligomeric states. Each copy of the second assembly further comprises a surface-exposed interface that interacts with a complementary surface-exposed interface on a trimeric assembly domain. As described in King et al., Bale et al., and patent publications WO2014124301 A1 and US20160122392 A1, the complementary interface between the trimeric assembly domain and second assembly domain drives the assembly of multiple copies of the trimeric assembly domain and second assembly domain to a target nanostructure. In various specific embodiments:
(a) when each first polypeptide is DS-Cav1-foldon-T33-31A (SEQ ID NO:69) or DS-Cav1-T33-31A (SEQ ID NO:70), each second polypeptide is T33-31B (SEQ ID NO:44);
(b) when each first polypeptide is DS-Cav1-foldon-T33-15B (SEQ ID NO:71) or DS-Cav1-T33-15B (SEQ ID NO:72), each second polypeptide is T33-15A (SEQ ID NO:45);
(c) when each first polypeptide is DS-Cav1-foldon-I53-50A (SEQ ID NO:73) or DS-Cav1-I53-50A (SEQ ID NO:74), each second polypeptide is I53-50B (SEQ ID NO:8), I53-50B.1 (SEQ ID NO:32), I53-50B.1NegT2 (SEQ ID NO:33), or I53-50B.4PosT1 (SEQ ID NO:34);
(d) when each first polypeptide is DS-Cav1-I32-28A (SEQ ID NO:75), each second polypeptide is I32-28B.

### Assembly of Full Valency Nanostructures by In Vitro Assembly of Two Components

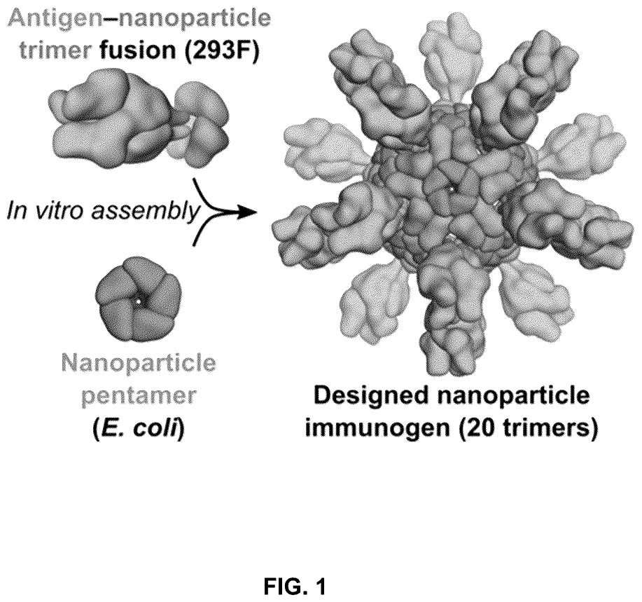

In some embodiments, each trimeric first assembly of the nanostructure bears an identical F protein as a genetic fusion; these nanostructures display the F protein at full (100%) valency. Such nanostructures are produced from purified first polypeptides and second polypeptides in a process called in vitro assembly. Purified trimeric first polypeptides comprising an F protein, are mixed with appropriate second polypeptides in an approximately 1:1 molar ratio in aqueous conditions (see FIG. 1). The second assembly interacts with the trimeric first assembly in order to drive assembly of the target nanostructure. Successful assembly of the target nanostructure can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled nanostructure can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the F protein in the nanostructure can be assessed by techniques commonly used to determine the identity of protein molecules in aqueous solutions, including but not limited to SDS-PAGE, mass spectrometry, protein sequencing, or amino acid analysis. The accessibility of the F protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, or anti-sera specific to the antigen.

### In Vitro Assembly of Partial Valency Nanostructures

In other embodiments, the nanostructures of the invention comprise one or more copies of trimeric first assemblies bearing F proteins as genetic fusions as well as one or more trimeric first assemblies that do not bear F proteins as genetic fusions; these nanostructures display the F proteins at partial valency. These partial valency nanostructures are produced by performing in vitro assembly with mixtures of first polypeptides in which the fraction of trimeric first assemblies bearing an F protein as a genetic fusion is equal to the desired valency of the antigen in the resulting nanostructure. The in vitro assembly reaction typically contains an approximately 1:1 molar ratio of total first polypeptides to total second polypeptides. By way of non-limiting example, performing an in vitro assembly reaction with a mixture of trimeric assemblies in which one half of the first polypeptides bear an F protein as a genetic fusion would yield an assembled nanostructure with an F protein valency of 50%. That is, 50% of the possible sites for F protein display on the nanostructure would be occupied. By way of non-limiting example, if the nanostructure is a 120-subunit assembly with icosahedral symmetry, the nanostructure comprises 20 total trimeric building blocks, and a 50% valency nanostructure displays 10 of the possible 20 F protein trimers. In this way, the ratio of F protein-bearing first polypeptides to first polypeptides lacking F proteins in an in vitro assembly reaction can be used to precisely tune the F protein valency of the resulting nanostructures. It will be understood by those of skill in the art that it is the average valency that can be tuned in this manner; the valency of individual nanostructures in the mixture will be a distribution centered around the average. Successful assembly of such partial valency nanostructures can be assessed using the techniques described above for evaluating full-valency nanostructures, and, if necessary, the partial valency nanostructures can be purified using the methods described for purifying full-valency nanostructures. The average valency of F protein-bearing first polypeptides in a given sample can be assessed by quantitative analysis using the techniques described above for evaluating the presence of F proteins in full-valency nanostructures.

### In Vitro Assembly of Nanostructures Co-Displaying Multiple F Proteins

In other embodiments, the nanostructures of the invention comprise two or more distinct first polypeptides bearing different F proteins as genetic fusions; these nanostructures co-display multiple different F proteins on the same nanostructure. These multi-antigen nanostructures are produced by performing in vitro assembly with mixtures of first polypeptides in which each first polypeptide bears one of two or more distinct F proteins as a genetic fusion. The fraction of each first polypeptide in the mixture determines the average valency of each F protein in the resulting nanostructures. The in vitro assembly reaction typically contains an approximately 1:1 molar ratio of total trimeric first polypeptides to total second polypeptides. The presence and average valency of each F protein-bearing first polypeptides in a given sample can be assessed by quantitative analysis using the techniques described above for evaluating the presence of F proteins in full-valency nanostructures.

In various embodiments, the nanostructures are between about 20 nanometers (nm) to about 40 nm in diameter, with interior lumens between about 15 nm to about 32 nm across and pore sizes in the protein shells between about 1 nm to about 14 nm in their longest dimensions.

In one embodiment, the nanostructure has icosahedral symmetry. In this embodiment, the nanostructure may comprise 60 copies of the first polypeptide and 60 copies of the second polypeptide. In one such embodiment, the number of identical first polypeptides in each first assembly is different than the number of identical second polypeptides in each second assembly. For example, in one embodiment, the nanostructure comprises twelve first assemblies and twenty second assemblies; in this embodiment, each first assembly may, for example, comprise five copies of the identical first polypeptide, and each second assembly may, for example, comprise three copies of the identical second polypeptide. In another embodiment, the nanostructure comprises twelve first assemblies and thirty second assemblies; in this embodiment, each first assembly may, for example, comprise five copies of the identical first polypeptide, and each second assembly may, for example, comprise two copies of the identical second polypeptide. In a further embodiment, the nanostructure comprises twenty first assemblies and thirty second assemblies; in this embodiment, each first assembly may, for example, comprise three copies of the identical first polypeptide, and each second assembly may, for example, comprise two copies of the identical second polypeptide. All of these embodiments are capable of forming synthetic nanomaterials with regular icosahedral symmetry. In various further embodiments, oligomeric states of the first and second polypeptides are as follows:
I53-34A: trimer+I53-34B: pentamer;
I53-40A: pentamer+I53-40B: trimer;
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer;
I52-32A: dimer+I52-32B: pentamer; and
I52-33A: pentamer+I52-33B: dimer

In another embodiment, the nanostructure of any embodiment or combination of embodiments of the invention has one or more of the following characteristics, each as demonstrated in the examples that follow:
(a) binds prefusion F-specific antibodies including but not limited to monoclonal antibody D25;
(b) forms a symmetrical structure, including but not limited to an icosahedral structure;
(c) is stable at 50° C.; and/or
(d) is stable in 2.25M guanidine hydrochloride.

In another aspect, the present invention provides isolated nucleic acids encoding a fusion protein of the present invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In a further aspect, the present invention provides recombinant expression vectors comprising the isolated nucleic acid of any embodiment or combination of embodiments of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In another aspect, the present invention provides host cells that have been transfected with the recombinant expression vectors disclosed herein, wherein the host cells can be either prokaryotic or eukaryotic, such as mammalian cells. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, NY). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In a further aspect, the invention provides an immunogenic composition comprising an effective amount of the nanostructure of any embodiment or combination of embodiments of the invention and a pharmaceutically acceptable carrier. The composition may comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The nanostructure may be the sole active agent in the composition, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen. Exemplary adjuvants include, but are not limited to, Adju-Phos™, Adjumer™, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL.TM., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

In another aspect, the invention provides methods for generating an immune response to paramyxovirus and/or pneumovirus F protein in a subject, comprising administering to the subject an effective amount of the immunogenic composition of any embodiment or combination of embodiments of the invention to generate the immune response. In a further aspect, the invention provides methods for treating or preventing a paramyxovirus and/or pneumovirus infection in a subject, comprising administering to the subject an effective amount of the immunogenic composition of any embodiment or combination of embodiments of the invention, thereby treating or preventing paramyxovirus and/or pneumovirus infection in the subject.

In one embodiment, the paramyxovirus and/or pneumovirus comprises respiratory syncytial virus. “Respiratory Syncytial Virus” and “RSV” refer to a negative-sense, single-stranded RNA virus that causes a respiratory disease, especially in children. When the method comprises treating an RSV infection, the immunogenic compositions are administered to a subject that has already been infected with the RSV, and/or who is suffering from symptoms (including but not limited to lower respiratory tract infections, upper respiratory tract infections, bronchiolitis, pneumonia, fever, listlessness, diminished appetite, recurrent wheezing, and asthma) indicating that the subject is likely to have been infected with the RSV. As used herein, “treat” or “treating” includes, but is not limited to accomplishing one or more of the following: (a) reducing paramyxovirus and/or pneumovirus titer in the subject; (b) limiting any increase of paramyxovirus and/or pneumovirus titer in the subject; (c) reducing the severity of paramyxovirus and/or pneumovirus symptoms; (d) limiting or preventing development of paramyxovirus and/or pneumovirus symptoms after infection; (e) inhibiting worsening of paramyxovirus and/or pneumovirus symptoms; (f) limiting or preventing recurrence of paramyxovirus and/or pneumovirus symptoms in subjects that were previously symptomatic for paramyxovirus and/or pneumovirus infection; and/or promoting maternal transmission of paramyxovirus and/or pneumovirus antibodies to infants (after maternal immunization).

When the method comprises limiting a paramyxovirus and/or pneumovirus infection, the immunogenic compositions are administered prophylactically to a subject that is not known to be infected, but may be at risk of exposure to the paramyxovirus and/or pneumovirus. As used herein, “limiting” means to limit RSV infection in subjects at risk of RSV infection. Groups at particularly high risk include children under age 18 (particularly infants 3 years or younger), adults over the age of 65, and individuals suffering from any type of immunodeficiency.

As used herein, an “effective amount” refers to an amount of the immunogenic composition that is effective for treating and/or limiting RSV infection. The immunogenic compositions are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 ug/kg-100 mg/kg body weight of the F protein or antigenic fragment thereof. The composition can be delivered in a single bolus, or may be administered more than once (e.g., 2, 3, 4, 5, or more times) as determined by attending medical personnel.

In one embodiment, the administering results in production of paramyxovirus and/or pneumovirus neutralizing antibodies in the subject. In another embodiment, the neutralizing antibodies are present in sera of the subject at a titer (1/ID₅₀) of at least 1,000; in other embodiments, the neutralizing antibodies are present in sera of the subject at a titer of 2,000 or 5,000.

### EXAMPLES

### METHODS

### Expression and Screening of Trimeric Building Blocks Comprising an F Protein and a Trimeric Assembly Domain

Human codon-optimized sequences for trimeric building blocks including and lacking DS-Cav1 fusions were ordered from Genscript. Building blocks for single-component nanostructures (i.e., I3-01) were cloned into the pcDNA3.1 vector (ThermoFisher Scientific) containing one CMV promoter, while building blocks for two-component nanostructures (e.g., I53-50) were cloned into the pBudCE4.1™ vector (ThermoFisher Scientific) containing both CMV and EF-1α promoters. Recombinant proteins were expressed by transient transfection of Expi293F™ cells (ThermoFisher Scientific) using polyethylenimine (PEI). Cell cultures were harvested five days post-transfection by centrifugation. Secreted proteins were analyzed by ELISA, using either direct coating of the cell supernatants or by sandwich ELISA. Briefly, 96-well MaxiSorp™ plates (Nunc) were coated with cell supernatant for direct ELISA or murine anti-His tag monoclonal antibody (ThermoFisher Scientific) for sandwich ELISA. Secreted proteins were detected using the human Palivizumab, MPE8, RSD5, and D25 monoclonal antibodies. Transfected Expi293F cells were fixed and permeabilized with BD cytofix/cytoperm (BD Biosciences), incubated with human Palivizumab, MPE8, and D25 monoclonal antibodies, and stained with Alexa Fluor 647-conjugated anti-human IgG antibody (Jackson ImmunoResearch). Stained cells were counted with a FACS Fortessa™ flow cytometer (BD Biosciences). Analysis was performed with FlowJo™ software. Cell lines were routinely tested for mycoplasma contamination.

### Expression and Purification of DS-Cav1-I53-50A

Lentivirus was produced by transient transfection of 293T (ATCC) cells using linear 25-kDa polyethyleneimine (PEI; Polysciences). Briefly, 4×10⁶ cells were plated onto 10 cm tissue culture plates. After 24 h, 3 μg of psPAX2, 1.5 μg of pMD2G (Addgene™ plasmid #12260 and #12259, respectively) and 6 μg of lentiviral vector plasmid were mixed in 500 μl diluent (5 mM HEPES, 150 mM NaCl, pH=7.05) and 42 μl of PEI (1 mg/ml) and incubated for 15 min. The DNA/PEI complex was then added to the plate drop-wise. Lentivirus was harvested 48 h post-transfection and concentrated 100-fold by low-speed centrifugation at 8000 g for 18 h. Transduction of the target cell line was carried out in 125 mL shake flasks containing 10×10⁶ cells in 10 mL of growth media. 100 uL of 100× lentivirus was added to the flask and the cells were incubated with shaking (225 rpm) at 37° C., in 8% CO₂ for 4-6 h. 20 mL of growth media was added to the shake flask after 4-6 h.

Transduced cells were expanded every other day to a density of 1×10⁶ cells/ml until a final culture size of 4 L was reached. The media was harvested after 17 days of total incubation after measuring final cell concentration (˜5×10⁶ cells/mL) and viability (˜90% viable). Culture supernatant was harvested by low-speed centrifugation to remove cells from the supernatant. NaCl and NaN₃ were added to final concentrations of 250 mM and 0.02%, respectively. The supernatant was loaded over one 5 mL HisTrap™ FF Crude column (GE Healthsciences) at 5 ml/min by an AKTA Pure™ (GE Healthsciences). The nickel elution was applied to a HiLoad™ 16/600 Superdex 200 pg column (GE Healthsciences) to further purify the target protein by size-exclusion chromatography. The size-exclusion purified target protein was snap frozen in liquid nitrogen and stored at −80° C.

### In Vitro Assembly of DS-Cav1-Bearing Nanostructures

100% valency particles (20 DS-Cav1 trimers per icosahedral nanostructure) were prepared by mixing DS-Cav1-foldon-I53-50A trimers and I53-50B.4PT1 pentamers at 50 μM each and incubating with rocking overnight at 4° C. In some cases, assembled nanostructures were purified from excess components remaining in the in vitro assembly reaction using a GE Sephacryl S-500 HR 16/60 column in a buffer comprising 25 mM Tris pH 8, 250 mM NaCl, 5% glycerol. Sample load and SEC fractions were analyzed by SDS-PAGE in the presence and absence of reducing agent. Peak fractions were pooled, concentrated using a GE Vivaspin™ 20 30kDa MWCO centrifugal filter, and quantified using an Agilent 8454 spectrophotometer.

66% valency particles (˜14 DS-Cav1 trimers per icosahedral nanostructure) were prepared by mixing DS-Cav1-foldon-I53-50A trimers, I53-50A trimers, and I53-50B.4PosT1 pentamers at 50, 25, and 75 μM, respectively. 33% valency particles (˜7 DS-Cav1 trimers per icosahedral nanostructure) were prepared by mixing DS-Cav1-foldon-I53-50A trimers, I53-50A trimers, and I53-50B.4PosT1 pentamers at 25, 50, and 75 μM, respectively. The in vitro assembly reactions were allowed to incubate with rocking overnight at 4° C. In some cases, assembled nanostructures were purified from excess components remaining in the in vitro assembly reaction using a GE Sephacryl™ S-500 HR 16/60 column in a buffer comprising 25 mM Tris pH 8, 250 mM NaCl, 5% glycerol. Sample load and SEC fractions were analyzed by SDS-PAGE in the presence and absence of reducing agent. Peak fractions were pooled, concentrated using a GE Vivaspin™ 20 30 kDa MWCO centrifugal filter, and quantified using an Agilent 8454 spectrophotometer after centrifuging at ˜21,000 g for 10 minutes at 4° C. Samples were then transferred to cryogenic tubes in 1 mL aliquots at 1.1 mg/mL for the 33% valency particles and 0.6 mg/mL for the 66% valency particles, flash frozen in liquid nitrogen, and stored at −80° C.

### Electron Microscopy of DS-Cav1-Bearing Nanostructures

Samples were prepared for negative stain EM by diluting to 0.01 mg/mL using 25 mM Tris pH 8, 250 mM NaCl, 5% glycerol and 3.5 μL was incubated on a glow-discharged, copper, carbon-coated grid for 20 seconds before blotting away the liquid with a piece of Whatman No. 1 filter paper. Within seconds of blotting away the sample, a 3.5 μL droplet of stain (2% w/v uranyl formate) was deposited and blotted away immediately, and then a second cycle of staining/blotting was performed.

### Circular Dichroism (CD) Spectropolarimetry

CD spectra from F proteins (0.5 mg ml⁻¹) were recorded on a Chirascan™ spectropolarimeter (Applied Photophysics) over the wavelength range of 195 to 260 nm at a bandwidth of 1 nm, step size of 0.5 nm, and 1 s per step. The spectra in the far-ultraviolet region required an average of three scans and were subtracted from blank spectra performed with buffer. Thermal denaturation was monitored by performing scans at intervals of 1° C., after equilibration for 1 min at each temperature. Data were fitted to a simple first order curve. The values of ΔA222 are represented on the y axis as the percentage of the values recorded at 20° C.

### Enzyme-Linked Immunosorbent Assay (ELISA)

To test specific binding of antibody or sera, 96-well MaxiSorp™ plates (Nunc) were coated with serial dilutions of tissue culture supernatants from cells expressing trimeric building blocks comprising F proteins and a trimeric assembly domain or 2 μg ml⁻¹ of the following purified proteins: Ds-Cav1 with foldon, Ds-Cav1 fused to a trimeric first polypeptide or DS-Cav1-displaying nanostructures. Plates were blocked with 1% bovine serum albumin (BSA) and incubated with titrated antibodies (D25, MPE8, Palivizumab, RSD5) or murine sera followed by AP-conjugated goat anti-human IgG (Southern Biotech, 2040-04) or goat anti-mouse IgG (Southern Biotech, 1030-04). Plates were then washed with PBS buffer (Gibco, Invitrogen), 0.05% Tween-20 and substrate (p-NPP, Sigma) was added and plates were read at 405 nm.

### Surface Plasmon Resonance (SPR)

The experiments were carried out at 25° C. on a ProteON™ XPR-36 instrument (Bio-Rad Laboratories) in a PBS buffer (Gibco, Invitrogen), 0.05% Tween-20. The D25 mAb was immobilized on a GLM sensor chip surface through amine coupling at 1000 response units (RU) and a blank surface with no protein was created under identical coupling conditions for use as a reference. Monoclonal antibodies (D25, MPE8, Palivizumab and 131-2a) were injected at a flow rate of 100 μl/min, at concentrations of 50 nM in different sensor channels. The data were processed using Proteon software and double referenced by subtraction of the blank surface and buffer only injection before local fitting of the data.

### Vaccination and Serological Analysis

Female BALB/c mice 6-9 weeks of age were obtained from ENVIGO Laboratories (Italy). All proteins were formulated with AddaVax™ adjuvant (Invivogen) according to the manufacturer's instruction. Mice were immunized subcutaneously (s.c) with a total protein dose corresponding to 5 μg of the DS-Cav1 antigen equivalent on day 0, 14, and 28 in 50% AddaVax™ in PBS. Mice were bled on day 24 and 40. Recovered sera were used to measure binding and neutralizing titers. Binding titers were measured by coating 3 μg/ml of DS-Cav1, I53-50 nanostructures or I53-50 nanostructure subunits.

### Virus Neutralization Assay and Microscopy Analysis

Neutralization of RSV infection by sera was measured using a micro-neutralization flow cytometry-based assay. Serial dilutions of sera were pre-incubated with RSV for 1 hour at 37° C. and added to 10000 HEp-2 (ATCC® CCL-23™) cells/well in 96-well flat-bottom plates (MOI of 1). After 24 hours, cells were washed, detached and fixed with 2% formaldehyde. Percentage of GFP positive cells were measured by High throughput FACS with an Intellicyt coupled to an automated platform. The Tissue Culture Inhibiting Dilution (TCID) neutralizing 50% of the Infection (TCID₅₀) was calculated by nonlinear regression with Prism 7 (GraphPad Software).

### Non-Human Primate (NHP) Immunization

Rhesus macaques were immunized i.m. (right quadriceps) at weeks 0 and 4 with trimeric DS-Cav1 (50 μg; n=4) or DS-Cav1-foldon-I53-50 nanostructures (96 μg, comprising 50 μg of displayed DS-Cav1; n=5) formulated in the MF59-like adjuvant SWE. Sera were obtained at weeks 6 and 16 for serological analysis.

### Stability of DS-Cav1-Bearing Nanostructures by Relative Binding to D25

Experiments were carried out at 20° C. on a ProteON™ XPR-36 instrument (Bio-Rad Laboratories) in a PBS buffer (Gibco, Thermo Fisher Scientific) and 0.05% Tween-20 (Sigma). 100 nM D25 antibody was immobilized on a GLM sensor chip surface through amine coupling (EDC/NHS chemistry) and a blank surface with no antibody was created under identical coupling conditions for use as a reference. Analyte proteins (soluble DS-Cav1, soluble DS-Cav1-I53-50A and DS-Cav1-foldon-I53-50 nanostructures), heat stressed at different temperatures (20, 50, 70 or 80° C.) for 1 h, were injected at a flow rate of 100 μl/min, at a concentration of 50 nM in the different sensor channels. Data were processed using Proteon software and double referenced by subtraction of the blank surface and buffer-only injection before local fitting of the data.

### Chemical Denaturation of Nanostructure-Related Proteins

Trimeric DS-Cav1, DS-Cav1-I53-50A, DS-Cav1-I53-50, I53-50, trimeric I53-50A, or pentameric I53-50B.4PT1 was diluted to a final concentration of 2.5 μM in 25 mM Tris pH 8, 250 mM NaCl, 5% glycerol with varying concentrations of guanidine hydrochloride, ranging from 0 M to 6.5 M, increasing in 0.25 M increments. Samples were prepared in triplicate and incubated for 16 hours at ambient temperature. On a Cary Eclipse Fluorescence Spectrophotometer, intrinsic fluorescence was measured for each guanidine hydrochloride concentration of each protein and of each replicate. A Peltier controller was used in the cell holder to maintain a temperature of 25° C. throughout all experiments. Using a 10 mm cell (Agilent Cuvette, part #6610021600), fluorescence spectra were collected, exciting at 290 nm and scanning emission from 310 nm to 510 nm at a rate of 60 nm/minute with a bandpass of 1 nm.

### Statistical Analysis

No statistical methods were used to predetermine sample size. Data were analyzed with Prism 6 (GraphPad™ Software) using the two-tailed non-parametric Mann-Whitney U test for two groups' comparison, or Kruskall-Wallis test (and Dunn's posttest) when three or more groups were compared.

### RESULTS

### Trimeric Building Blocks Comprising an F Protein and a Trimeric Assembly Domain

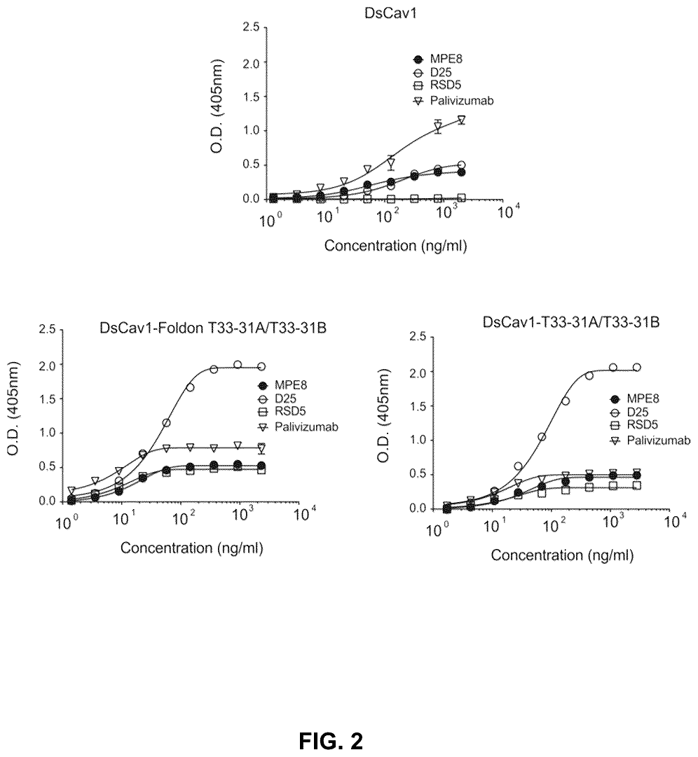

Several trimeric building blocks, each comprising an F protein genetically fused to a trimeric assembly domain, were found to be secreted from HEK293F cells with their F proteins in a well-folded, prefusion conformation as judged by prefusion-specific monoclonal antibody binding in ELISA assays. FIG. 2 shows an example of ELISA data analyzing the supernatant of HEK293F cells expressing DS-Cav1-foldon, DS-Cav1-foldon-T33-31A, and DS-Cav1-T33-31A. Several other trimeric building blocks yielded detectable secretion of well-folded, prefusion F proteins.

### Expression and Purification of DS-Cav1-Foldon-I53-50A

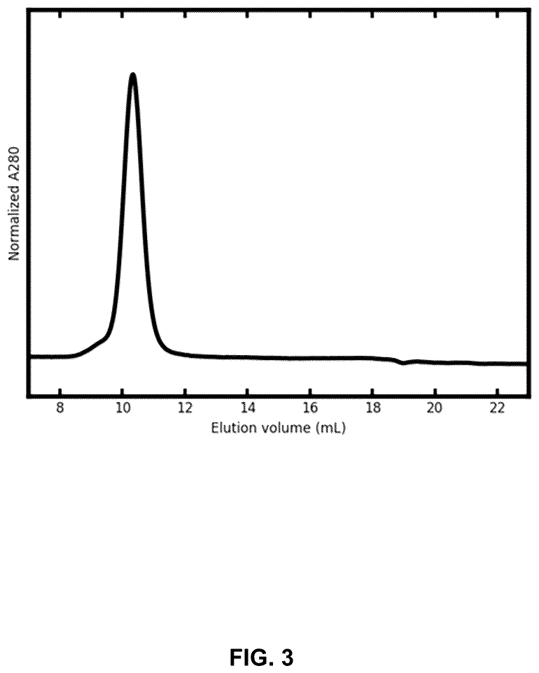

A lentiviral vector encoding DS-Cav1-foldon-I53-50A was used to transduce HEK293F cells for large-scale expression. The secreted protein was purified from tissue culture supernatants by immobilized metal affinity chromatography and size exclusion chromatography. Size exclusion chromatograms (FIG. 3) indicated that the purified protein formed a single, monodisperse species.

### Expression and Purification of I53-50B.4PT1

I53-50B.4PT1, a pentameric protein comprising a second assembly domain that interacts with the trimeric assembly domain in I53-50A or DS-Cav1-foldon-I53-50A to drive assembly of icosahedral I53-50-based nanostructures, was expressed and purified as described in Bale et al. and patent publication US20160122392 A1.

### In Vitro Assembly and Characterization of DS-Cav1-Bearing I53-50 Nanostructures

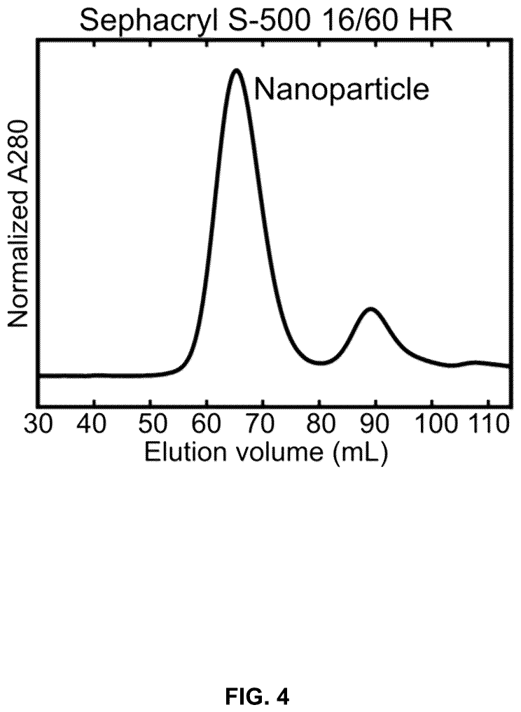

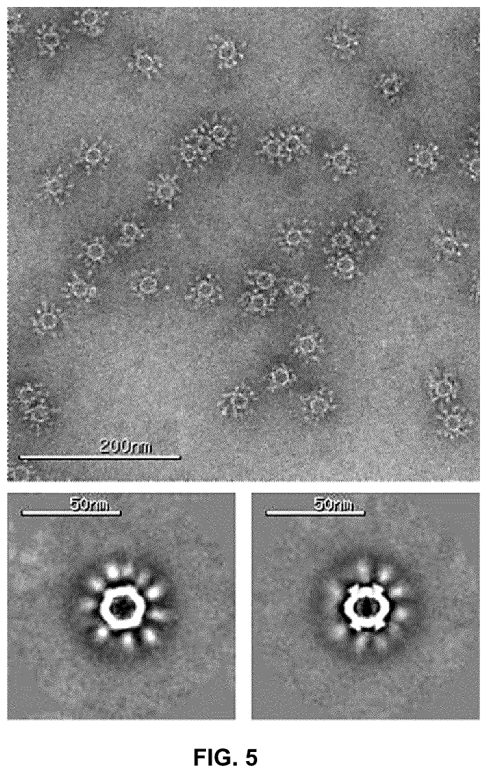

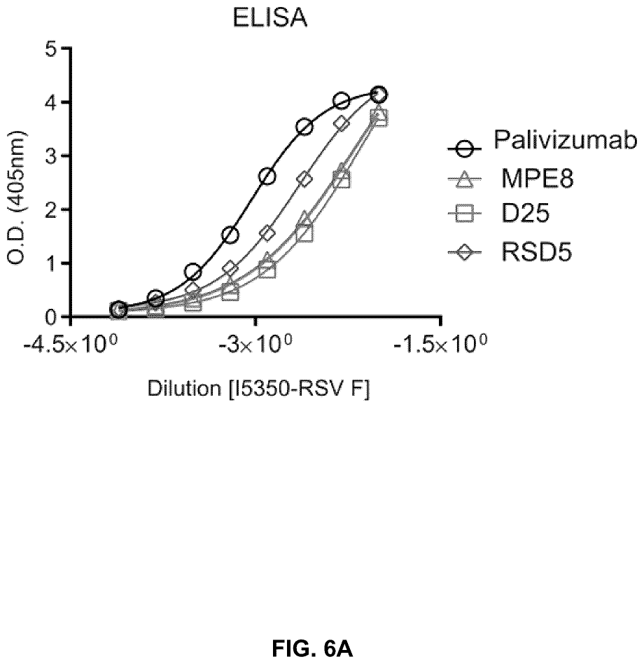

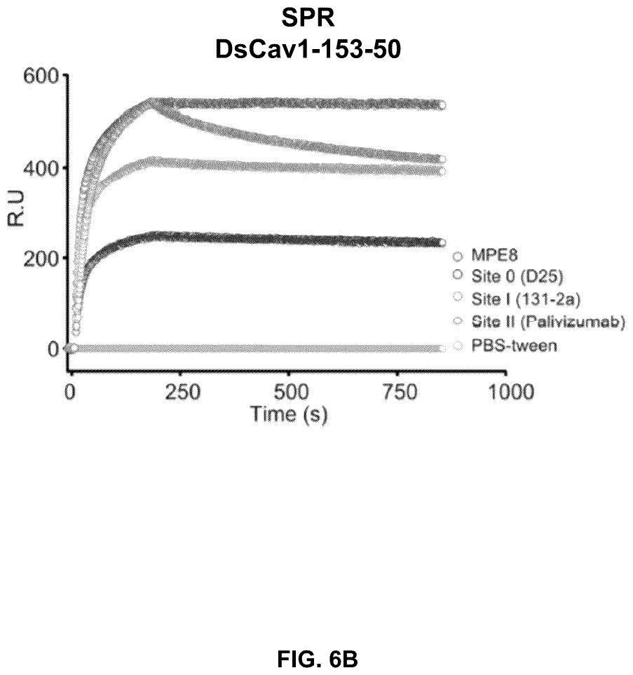

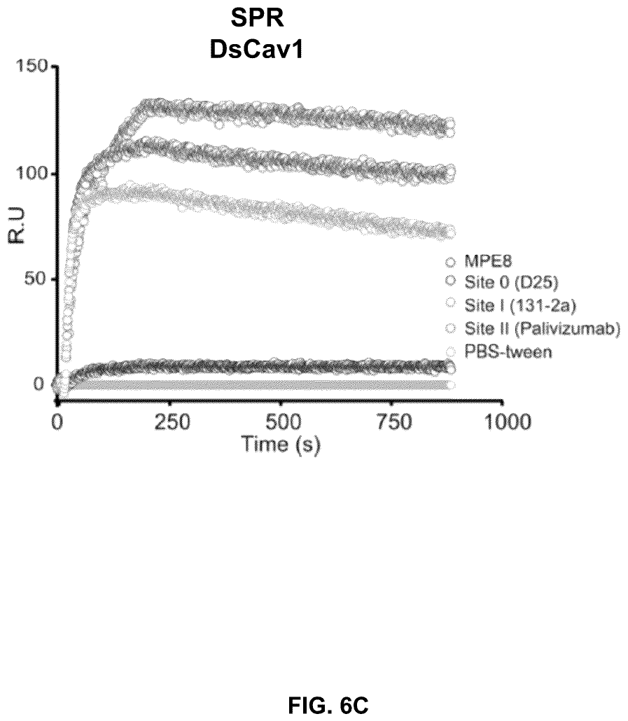

I53-50 is a 120-subunit two-component nanostructure with icosahedral symmetry comprising 20 trimeric (I53-50A) and 12 pentameric (I53-50B) building blocks, as recently described by Bale et al. The N terminus of I53-50A is exposed on the exterior of the I53-50 nanostructure, which enables the display of antigens on the nanostructure exterior through genetic fusion to the I53-530A N terminus. Purified DS-Cav1-foldon-I53-50A and I53-50B.4PT1 were assembled in vitro to form 120-subunit icosahedral nanostructures displaying various amounts of DS-Cav1 on the nanostructure exteriors by mixing the two purified proteins in various molar ratios. In separate preparations, nanostructures displaying DS-Cav1 at valencies of 100% (20 trimers), 66% (˜14 trimers), and 33% (˜7 trimers) were prepared as described above. The species present in the in vitro assembly reactions after overnight incubation were assessed by several techniques, including size exclusion chromatography-multi-angle light scattering (SEC-MALS), dynamic light scattering, and UV/vis spectroscopy. Assembled, 120-subunit nanostructures were purified from the in vitro assembly reactions using size exclusion chromatography (an example chromatogram obtained using the 100% valency nanostructures is presented in FIG. 4). The purified nanostructures were characterized by negative stain electron microscopy, which revealed fields of monodisperse particles in which DS-Cav1 was clearly visible as spikes projecting outward from the core icosahedral I53-50 assembly (an example micrograph obtained using the 100% valency particles is presented in FIG. 5). ELISA assays using monoclonal antibodies specific to the prefusion conformation confirmed that the DS-Cav1 thus displayed on the nanostructure exteriors was well-folded and antigenically intact (FIG. 6). Surface plasmon resonance experiments evaluating the kinetics of monoclonal antibody binding revealed that antibody dissociation from the 100% valency DS-Cav1-foldon-I53-50 nanostructures was slower than from DS-Cav1-foldon trimers, likely due to avidity effects deriving from the multivalent presentation of DS-Cav1 on the nanostructure exterior (FIG. 6). Together, these experiments confirmed that the DS-Cav1-foldon-I53-50 nanostructures formed monodisperse, icosahedral nanostructures that display well-folded, antigenically intact DS-Cav1 trimers on their exteriors. These findings motivated experiments to evaluate the utility of the DS-Cav1-foldon-I53-50 nanostructures as immunogens for inducing humoral immune responses against DS-Cav1 in animals.

### Immunogenicity of DS-Cav1-Foldon-I53-50 Nanostructures

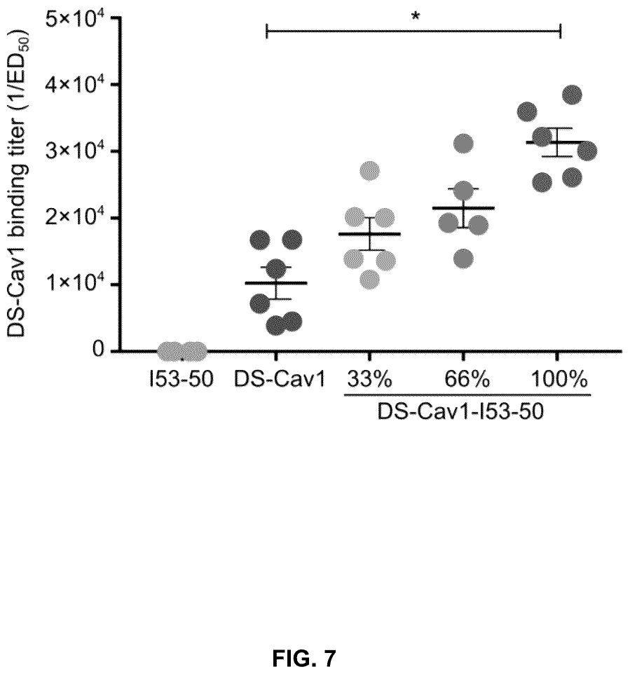

The DS-Cav1-foldon-I53-50 nanostructures displaying DS-Cav1 at 33%, 66%, and 100% valency were injected into mice using a prime-boost strategy as described above. Additional groups of mice were injected with trimeric DS-Cav1-foldon as a benchmark for the humoral immune response induced against DS-Cav1 by the nanostructures or I53-50 nanostructures lacking displayed DS-Cav1 as negative controls for a DS-Cav1 specific response. ELISA assays of serum extracted from the mice at defined time points after the injections were used to measure DS-Cav1 specific antibody titers present in the sera of the injected animals (FIG. 7). As expected, sera from animals injected with the I53-50 nanostructures lacking displayed DS-Cav1 did not contain antibodies specific to DS-Cav1. Trimeric DS-Cav1-foldon induced DS-Cav1-specific antibodies, in accordance with previous results (McClellan et al.). The 33%, 66%, and 100% valency DS-Cav1 nanostructures all induced higher DS-Cav1-specific antibody titers than trimeric DS-Cav1-foldon, with the antibody titers increasing with increasing DS-Cav1 valency. DS-Cav1-specific titers were roughly 2.5-fold higher on average in mice injected with 100% valency DS-Cav1-foldon-I53-50 nanostructures compared to DS-Cav1. These results demonstrate that immunogens in which paramyxovirus F proteins are multivalently displayed on self-assembling protein nanostructures can induce higher humoral immune responses when injected into animals.

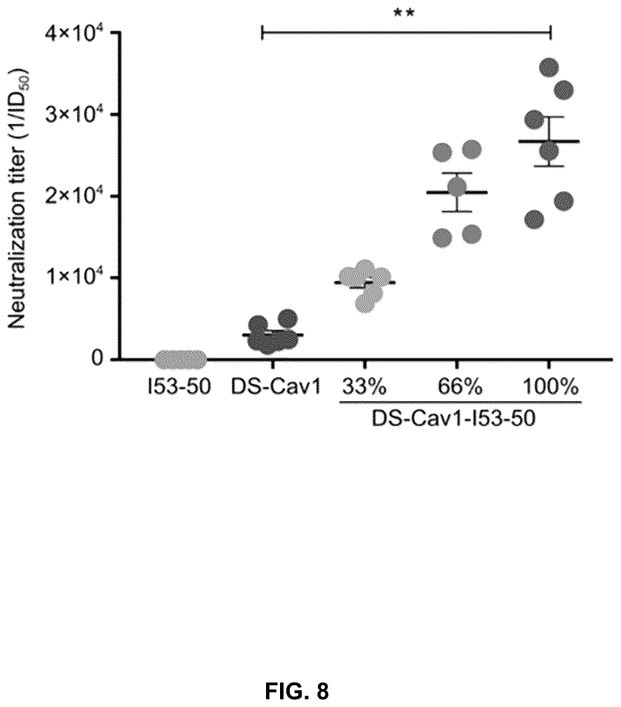

The sera from the mice injected with the series of immunogens described above was also evaluated for the presence of neutralizing antibody titers using the standard neutralization assay in HEp-2 cells (FIG. 8). The trend in serum neutralizing antibody titers correlated highly with the trend observed in DS-Cav1-specific binding antibody titers. Sera from animals injected with the I53-50 nanostructures lacking displayed DS-Cav1 did not neutralize virus, consistent with the lack of DS-Cav1-specific antibodies in these sera. The sera from animals injected with trimeric DS-Cav-1-foldon neutralized virus with an average titer (1/1D₅₀) of 3,030. The 33%, 66%, and 100% valency DS-Cav1-I53-50 nanostructures induced higher neutralizing antibody titers than trimeric DS-Cav1-foldon, with average titers of 9,400, 20,000, and 30,500, respectively. These results demonstrate that the higher humoral response induced by immunogens in which paramyxovirus F proteins are multivalently displayed on self-assembling protein nanostructures result in more effective virus neutralization.

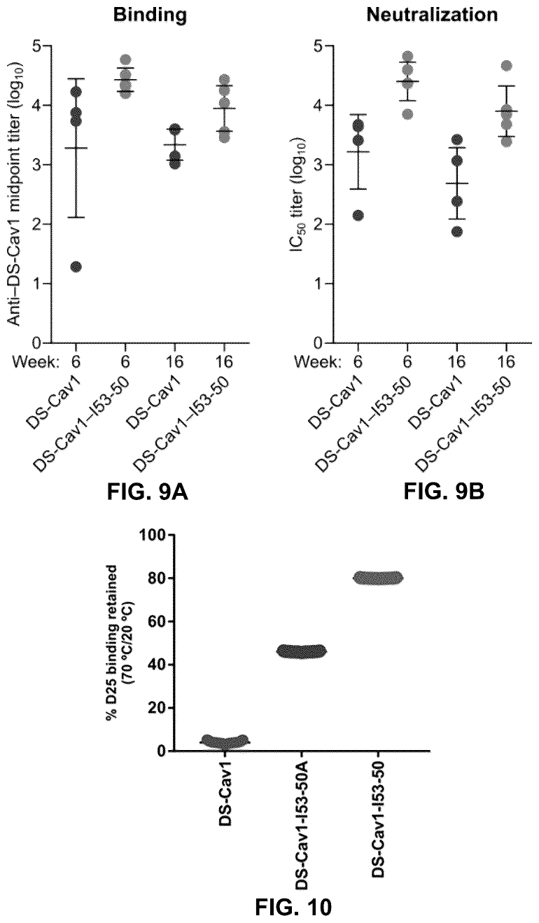

The DS-Cav1-foldon-I53-50 nanostructures were also injected into Rhesus macaques to evaluate their immunogenicity in a primate immune system. The animals were injected intramuscularly at weeks 0 and 4 with either free DS-Cav1 trimer or DS-Cav1-foldon-I53-50 nanostructures displaying DS-Cav1 at 100% valency. In both cases, the dose of DS-Cav1 antigen was 50 μg, and the immunogens were formulated with the MF59-like, squalene-based oil-in-water emulsion adjuvant SWE. Sera obtained from the animals at weeks 6 and 16 were evaluated for anti-DS-Cav1 antibody titers and RSV-neutralizing antibody titers (FIG. 9). The results mirrored those obtained in mice. At week 16, the mean anti-DS-Cav1 antibody titer was 4-fold higher in animals injected with the DS-Cav1-foldon-I53-50 nanostructure compared to animals injected with trimeric DS-Cav1. The mean RSV-neutralizing antibody titer at week 16 was 16-fold higher in animals injected with the DS-Cav1-foldon-I53-50 nanostructure compared to animals injected with trimeric DS-Cav1. These results demonstrate, in a primate immune system, that immunogens in which paramyxovirus F proteins are multivalently displayed on self-assembling protein nanostructures induce more robust humoral immune responses, including high levels of virus-neutralizing antibodies, than the trimeric paramyxovirus F proteins alone.

### Physical Stabilization of DS-Cav1 by Fusion to I53-50A

Given the key antigenic properties of prefusion F, we used two orthogonal approaches to measure the physical stability of DS-Cav1 when fused to I53-50A and/or when further assembled into the icosahedral nanostructure. The first assay measured the retention of binding by a prefusion-specific mAb (D25) after thermal stress, an approach that has been used previously to characterize prefusion F stability (McLellan et al. 2013; Joyce et al. 2016; Krarup et al. 2015). Samples of trimeric DS-Cav1, trimeric DS-Cav1-I53-50A, and DS-Cav1-I53-50 nanostructures containing equivalent concentrations (50 nM) of DS-Cav1 were split into four aliquots and incubated at 20, 50, 70 or 80° C. for 1 hour. After cooling to room temperature, D25 binding was assayed by surface plasmon resonance (SPR). We found that all samples bound D25 equivalently at 20 and 50° C., but lost most of their reactivity to D25 after 1 hour at 80° C. as previously reported for DS-Cav1 (McLellan et al. 2013; Joyce et al. 2016) (FIG. 10). Interestingly, while D25 was also unable to bind trimeric DS-Cav1 incubated at 70° C. for 1 hour, trimeric DS-Cav1-I53-50A and the DS-Cav1-I53-50 nanostructures retained 50 and 80% of their respective binding signals (FIG. 10). While the multivalent nature of the DS-Cav1-I53-50 nanostructures complicates direct quantitative comparisons to trimeric DS-Cav1, these results indicate that genetic fusion to the I53-50A trimer further stabilizes the prefusion conformation of DS-Cav1, and suggest that this increased stability is maintained in the context of the assembled nanostructure immunogen.

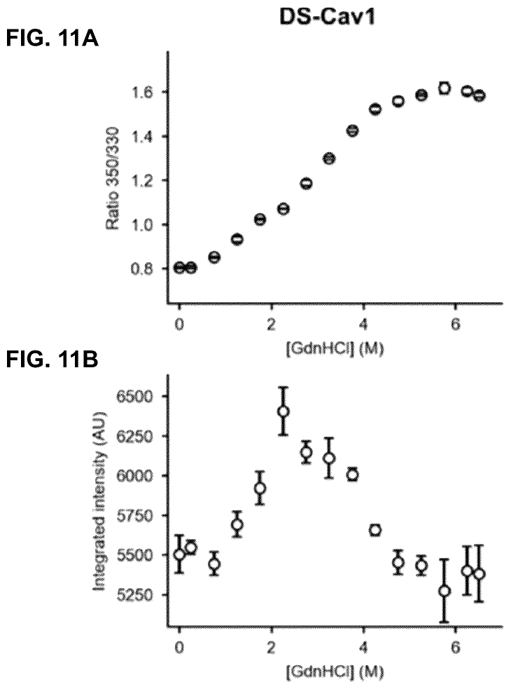

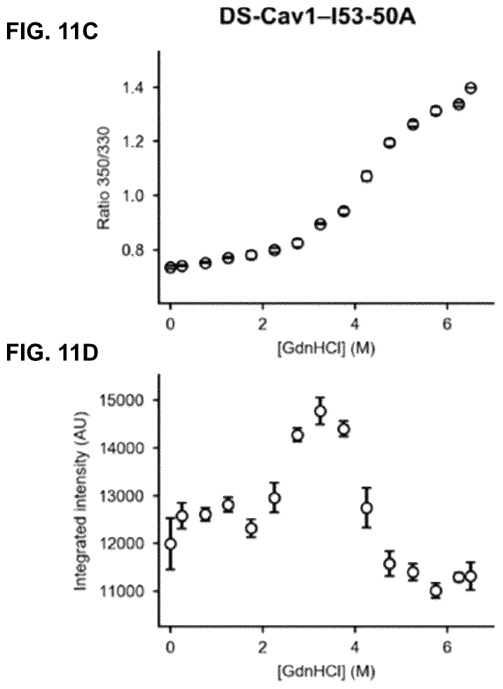

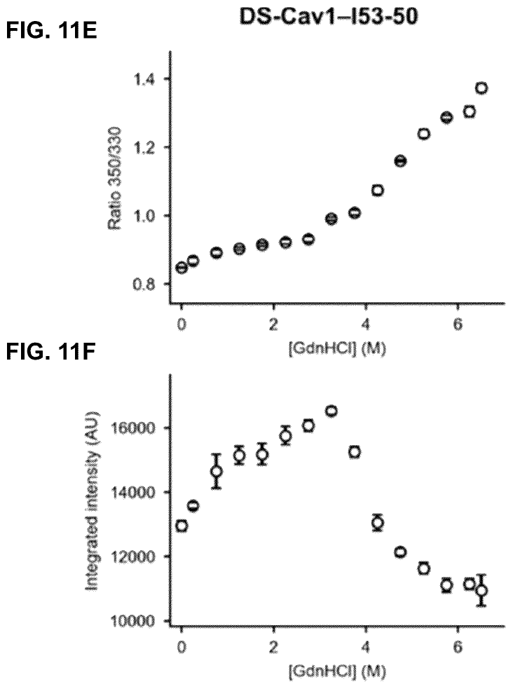

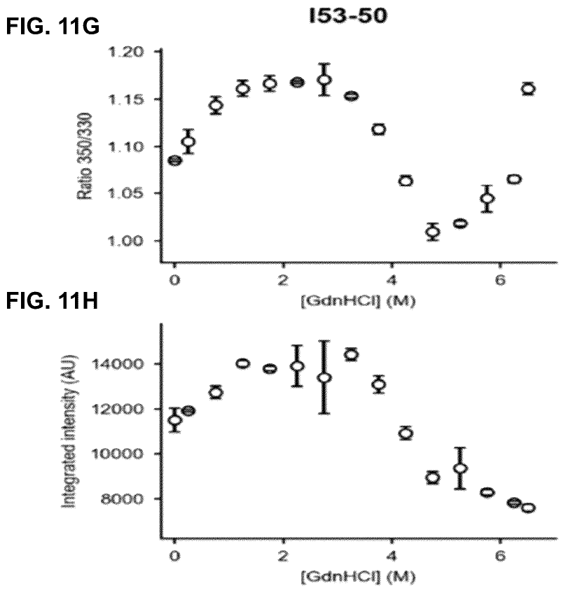

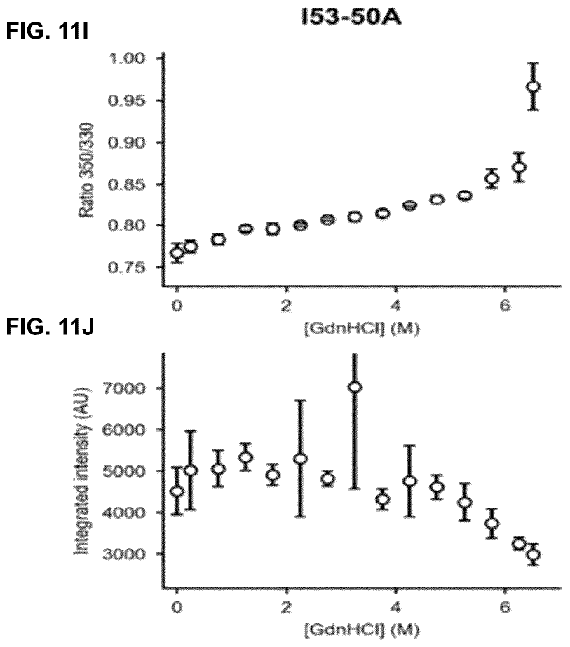

We used chemical denaturation in guanidine hydrochloride (GdnHCl), monitored by intrinsic tryptophan fluorescence, as a second, antibody-independent technique to evaluate physical stability. Analyzing fluorescence emission from DS-Cav1 incubated in 0-6.5 M GdnHCl revealed that the protein undergoes two subtly distinct transitions, one between 0.25 and 2.25 M GdnHCl and another between 2.25 and 5.75 M (FIG. 11). In contrast, only a single transition is apparent for trimeric DS-Cav1-I53-50A, occurring between 2.25 and 6.25 M GdnHCl (FIG. 11). It is unclear at present whether the transition at lower [GdnHCl] observed for DS-Cav1 is absent from trimeric DS-Cav1-I53-50A or simply shifted to higher [GdnHCl]. However, it is clear that the native conformation of DS-Cav1 is stabilized by genetic fusion to trimeric I53-50A, mirroring the results obtained by measuring D25 binding after thermal stress. Comparing the data for the DS-Cav1-I53-50 nanostructure and the I53-50 nanostructure alone (lacking fused DS-Cav1) indicated that the stabilization is maintained upon assembly to the icosahedral nanostructure (FIG. 11). The source of this effect is likely the extreme stability of the I53-50A trimer. I53-50A is derived from the KDPG aldolase of the hyperthermophilic bacterium T. maritima and only began to exhibit changes in fluorescence at very high 5.75 M) GdnHCl concentrations (FIG. 11).

We made addition constructs to assess the number of GS repeats and the need for a stabilization domain such as the Foldon moiety.

| IPD Name | MS (Da) | Construct Information |
|---|---|---|
| RSV_F-10 | 74005.38 | DS-Cav1-8GS-HelExt-50A |
| RSV_F-11 | 74293.64 | DS-Cav1-12GS-HelExt-50A |
| RSV_F-12 | 74551.87 | DS-Cav1-16GS-HelExt-50A |
| RSV_F-13 | 77212.97 | DS-Cav1-foldon-10GS-HelExt-50A |
| RSV_F-14 | 77558.28 | DS-Cav1-foldon-15GS-HelExt-50A |
| RSV_F-15 | 77933.62 | DS-Cav1-foldon-20GS-HelExt-50A |

Studies were based on expression yield in a small-scale transient transfection. Plasmids capable of expressing the relevant constructs were transformed into NEB 5α E. coli cells and selected on LB+carbenicillin agar plates. 1 mL cultures were prepared by inoculating TB media with a bacterial colony and again selecting with 50 ug/mL carbenicillin. A Qiagen Mini Prep kit was used to purify plasmid from the E. coli cultures in accordance with their protocol. Expi293F™ Cells (ThermoFisher) were cultured in Expi293™ Expression Medium (ThermoFisher) supplemented with penicillin (100 u/mL) and streptomycin (100 μg/mL) at 8% CO₂, 37° C., and 125 rpm shaking.

On the day prior to transfection, cells were seeded at a concentration of 2E6 cells/mL. On the day of transfection, cells were counted by a Countess II (ThermoFisher) with trypan blue to determine cell viability. Cell concentration was adjusted to 2.5E6 cells/mL, and cells where plated into untreated 12-well plates (Corning) in 1 mL volumes. 1 μg of DNA plasmid were transfected per each well using Expifectamine™ (ThermoFisher), following the manufacturer's directions. Enhancers, components of ThermoFisher's Expifectamine™ Transfection Kit, were added 18 hours after transfection. The 1 mL cultures were harvested 5 days post-transfection, and the cells were pelleted from the supernatant by centrifugation at 1,500×g for 5 minutes at 4° C. Supernatants were filtered through a 0.45 μM filter with a PVDF membrane.

Filtered supernatants containing DS-Cav1-I53-50A constructs were denatured and boiled for 10 minutes at 95° C. for 10 minutes in 2× Laemmli buffer with 2-mercaptoethanol. SDS-PAGE separated the sample fractions, which were then transferred to a nitrocellulose membrane and probed with palivizumab, followed with a secondary antibody, anti-human conjugated to HRP. Blot was imaged using Clarity Western ECL Blotting Substrate (Bio-Rad).

Filtered supernatants containing DS-Cav1-I53-50A constructs were bound to Nunc MaxiSorp™ 96-well plates in a two-fold dilution series. The pre-fusion conformation-specific antibody D25 was used to detect DS-Cav1-I53-50A, followed by a secondary anti-human antibody conjugated to HRP. Protein yield was determined colorimetrically via the substrate TMB and absorbances were collected at 450 nm.

The expression yields and binding of the prefusion-specific mAb D25 (data not shown) indicate that all constructs express well and are in the prefusion conformation. Those of skill in the art would have expected that a heterologous trimerization domain (such as the foldon) would be required for proper expression and folding of prefusion F constructs. Our results indicate that the I53-50A nanostructure component can support the expression and proper folding of DS-Cav1 without the use of a trimerization domain like the foldon. Binding of D25 to these constructs suggests that they are antigenically intact and would be expected to induce potent immune responses, including neutralizing antibodies, similarly to nanostructures comprising the DS-Cav1-foldon-I53-50 fusion polypeptide.

1. A nanostructure, comprising:
(a) a plurality of first assemblies, each first assembly comprising a plurality of identical first polypeptides;
(b) a plurality of second assemblies, each second assembly comprising a plurality of identical second polypeptides, wherein the second polypeptide differs from the first polypeptide;
wherein the plurality of first assemblies non-covalently interact with the plurality of second assemblies to form a nanostructure; and
wherein the nanostructure displays multiple copies of one or more paramyxovirus and/or pneumovirus F proteins, or antigenic fragments thereof, on an exterior of the nanostructure.

2. The nanostructure of claim 1, wherein:
(a) the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of a polypeptide selected from the group consisting of SEQ ID NOS:1-51; and
(b) the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of a polypeptide selected from the group consisting of SEQ ID NOS:1-51.

3. The nanostructure of claim 1, wherein the one or more paramyxovirus and/or pneumovirus F proteins, or antigenic fragments thereof, comprise a polypeptide having at least 75%, 80%, 85%, 90%, 91%, 92%, 93%, 94%, 95%, 96%, 97%, 98%, 99%, or 100% identity along its full length to a polypeptide selected from the group consisting of SEQ ID NOS:53, 61-68, and 101.

4. The nanostructure of claim 1, wherein the one or more paramyxovirus and/or pneumovirus F proteins, or antigenic fragments thereof, comprise a polypeptide having at least 75%, 80%, 85%, 90%, 91%, 92%, 93%, 94%, 95%, 96%, 97%, 98%, 99%, or 100% identity along its full length to an RSV F protein or mutant thereof selected from the group consisting of SEQ ID NO:53 and 61-64, wherein the polypeptide includes one or more of the following residues: 67I, 149C, 458C, 46G, 465Q, 215P, 92D, and 487Q.

5. The nanostructure of claim 1, wherein the one or more paramyxovirus and/or pneumovirus F proteins, or antigenic fragments thereof, comprise a polypeptide having at least 75%, 80%, 85%, 90%, 91%, 92%, 93%, 94%, 95%, 96%, 97%, 98%, 99%, or 100% identity along its full length to an MPV F protein or mutant thereof selected from the group consisting of SEQ ID NO:65-68 and 101, wherein the polypeptide includes one or more of the following residues: 113C, 120C, 339C, 160F, 177L, 185P, and 426C.

6. The nanostructure of claim 1, wherein the first polypeptides and the second polypeptides comprise polypeptides having at least 75%, 80%, 85%, 90%, 91%, 92%, 93%, 94%, 95%, 96%, 97%, 98%, 99%, or 100% identity along their full length to the amino acid sequences selected from the following pairs:
SEQ ID NO:1 and SEQ ID NO:2 (I53-34A and I53-34B);
SEQ ID NO:3 and SEQ ID NO:4 (I53-40A and I53-40B);
SEQ ID NO:3 and SEQ ID NO:24 (I53-40A and I53-40B.1);
SEQ ID NO:23 and SEQ ID NO:4 (I53-40A.1 and I53-40B);
SEQ ID NO:35 and SEQ ID NO:36 (I53-40A genus and I53-40B genus);
SEQ ID NO:5 and SEQ ID NO:6 (I53-47A and I53-47B);
SEQ ID NO:5 and SEQ ID NO:27 (I53-47A and I53-47B.1);
SEQ ID NO:5 and SEQ ID NO:28 (I53-47A and I53-47B.1NegT2);
SEQ ID NO:25 and SEQ ID NO:6 (I53-47A.1 and I53-47B);
SEQ ID NO:25 and SEQ ID NO:27 (I53-47A.1 and I53-47B.1);
SEQ ID NO:25 and SEQ ID NO:28 (I53-47A.1 and I53-47B.1NegT2);
SEQ ID NO:26 and SEQ ID NO:6 (I53-47A.1NegT2 and I53-47B);
SEQ ID NO:26 and SEQ ID NO:27 (I53-47A.1NegT2 and I53-47B.1);
SEQ ID NO:26 and SEQ ID NO:28 (I53-47A.1NegT2 and I53-47B.1NegT2);
SEQ ID NO:37 and SEQ ID NO:38 (I53-47A genus and I53-47B genus);
SEQ ID NO:7 and SEQ ID NO:8 (I53-50A and I53-50B);
SEQ ID NO:7 and SEQ ID NO:32 (I53-50A and I53-50B.1);
SEQ ID NO:7 and SEQ ID NO:33 (I53-50A and I53-50B.1NegT2);
SEQ ID NO:7 and SEQ ID NO:34 (I53-50A and I53-50B.4PosT1);
SEQ ID NO:29 and SEQ ID NO:8 (I53-50A.1 and I53-50B);
SEQ ID NO:29 and SEQ ID NO:32 (I53-50A.1 and I53-50B.1);
SEQ ID NO:29 and SEQ ID NO:33 (I53-50A.1 and I53-50B.1NegT2);
SEQ ID NO:29 and SEQ ID NO:34 (I53-50A.1 and I53-50B.4PosT1);
SEQ ID NO:30 and SEQ ID NO:8 (I53-50A.1NegT2 and I53-50B);
SEQ ID NO:30 and SEQ ID NO:32 (I53-50A.1NegT2 and I53-50B.1);
SEQ ID NO:30 and SEQ ID NO:33 (I53-50A.1NegT2 and I53-50B.1NegT2);
SEQ ID NO:30 and SEQ ID NO:34 (I53-50A.1NegT2 and I53-50B.4PosT1);
SEQ ID NO:31 and SEQ ID NO:8 (I53-50A.1PosT1 and I53-50B);
SEQ ID NO:31 and SEQ ID NO:32 (I53-50A.1PosT1 and I53-50B.1);
SEQ ID NO:31 and SEQ ID NO:33 (I53-50A.1PosT1 and I53-50B.1NegT2);
SEQ ID NO:31 and SEQ ID NO:34 (I53-50A.1PosT1 and I53-50B.4PosT1);
SEQ ID NO:39 and SEQ ID NO:40 (I53-50A genus and I53-50B genus);
SEQ ID NO:9 and SEQ ID NO:10 (I53-51A and I53-51B);
SEQ ID NO:11 and SEQ ID NO:12 (I52-03A and I52-03B);
SEQ ID NO:13 and SEQ ID NO:14 (I52-32A and I52-32B);
SEQ ID NO:15 and SEQ ID NO:16 (I52-33A and I52-33B)
SEQ ID NO:17 and SEQ ID NO:18 (I32-06A and I32-06B);
SEQ ID NO:19 and SEQ ID NO:20 (I32-19A and I32-19B);
SEQ ID NO:21 and SEQ ID NO:22 (I32-28A and I32-28B);
SEQ ID NO:23 and SEQ ID NO:24 (I53-40A.1 and I53-40B.1);
SEQ ID NO:41 and SEQ ID NO:42 (T32-28A and T32-28B);
SEQ ID NO:43 and SEQ ID NO:44 (T33-09A and T33-09B);
SEQ ID NO:45 and SEQ ID NO:46 (T33-15A and T33-15B);
SEQ ID NO:47 and SEQ ID NO:48 (T33-21A and T33-21B);
SEQ ID NO:49 and SEQ ID NO:50 (T33-28A and T32-28B); and
SEQ ID NO:51 and SEQ ID NO:44 (T33-31B and T33-09B (also referred to as T33-31B))

7. The nanostructure of claim 1, wherein the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-31A (SEQ ID NO:51) and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-09B/T33-31B (SEQ ID NO:44).

8. The nanostructure of claim 1, wherein the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-15B (SEQ ID NO:46) and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of T33-15A (SEQ ID NO:45).

9. The nanostructure of claim 1, wherein the first polypeptides comprise polypeptides having at least 75%, 80%, 85%, 90%, 91%, 92%, 93%, 94%, 95%, 96%, 97%, 98%, 99%, or 100% identity along their full length to the amino acid sequence selected from the group consisting of I53-50A (SEQ ID NO:7), I53-50A.1 (SEQ ID NO:29), I53-50A.1NegT2 (SEQ ID NO:30), and I53-50A.1PosT1 (SEQ ID NO:31), and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence selected from the group consisting of I53-50B (SEQ ID NO:8), I53-50B.1 (SEQ ID NO:32), I53-50B.1NegT2 (SEQ ID NO:33), and I53-50B.4PosT1 (SEQ ID NO:34).

10. The nanostructure of claim 1, wherein the first polypeptides comprise polypeptides having at least 75%, 80%, 85%, 90%, 91%, 92%, 93%, 94%, 95%, 96%, 97%, 98%, 99%, or 100% identity along their full length to the amino acid sequence of I32-28A (SEQ ID NO:21) and the second polypeptides comprise polypeptides having at least 75%, 80%, 85%, 90%, 91%, 92%, 93%, 94%, 95%, 96%, 97%, 98%, 99%, or 100% identity along their full length to the amino acid sequence of I32-28B (SEQ ID NO:22).

11. The nanostructure of claim 1, wherein the one or more paramyxovirus and/or pneumovirus F proteins, or antigenic fragments thereof, are expressed as a fusion protein with the first polypeptides.

12. The nanostructure of claim 11, wherein the plurality of first assemblies each comprise identical fusion proteins.

13. The nanostructure of claim 11, wherein the plurality of first assemblies in total comprise two or more paramyxovirus and/or pneumovirus F proteins, or antigenic fragments thereof.

14. The nanostructure of claim 11, wherein only a subset of the first polypeptides comprise a fusion protein with an F protein or antigenic fragment thereof.

15. The nanostructure of claim 1, wherein each first assembly comprises a homotrimer of the first polypeptide.

16. The nanostructure of claim 1, wherein the one or more paramyxovirus and/or pneumovirus F proteins, or antigenic fragments thereof comprises polypeptides having at least 75%, 80%, 85%, 90%, 91%, 92%, 93%, 94%, 95%, 96%, 97%, 98%, 99%, or 100% identity along their full length to the amino acid sequence the amino acid sequence of DS-Cav1 (SEQ ID NO:53).

17. The nanostructure of claim 11, wherein each fusion protein comprises an amino acid linker positioned between the first polypeptide and the one or more paramyxovirus and/or pneumovirus F proteins, or antigenic fragment thereof.

18. The nanostructure of claim 17, wherein the amino acid linker sequence comprises one or more trimerization domain.

19. The nanostructure of claim 17, wherein the amino acid linker sequence comprises the amino acid sequence GYIPEAPRDGQAYVRKDGEWVLLSTFL (SEQ ID NO:54).

20. The nanostructure of claim 17, wherein the amino acid linker sequence comprises a Gly-Ser linker.

21. The nanostructure of claim 11, wherein the fusion protein comprises polypeptides having at least 75%, 80%, 85%, 90%, 91%, 92%, 93%, 94%, 95%, 96%, 97%, 98%, 99%, or 100% identity along their full length to the amino acid sequence selected from the group consisting of SEQ ID NOS:69-100.

22. The nanostructure of claim 21, wherein:
(a) when each fusion protein is DS-Cav1-foldon-T33-31A (SEQ ID NO:69) or DS-Cav1-T33-31A (SEQ ID NO:70), each second polypeptide is T33-31B (SEQ ID NO:44);
(b) when each fusion protein is DS-Cav1-foldon-T33-15B (SEQ ID NO:71) or DS-Cav1-T33-15B (SEQ ID NO:72), each second polypeptide is T33-15A (SEQ ID NO:45);
(c) when each fusion protein is DS-Cav1-foldon-I53-50A (SEQ ID NO:73) or DS-Cav1-I53-50A (SEQ ID NO:74), each second polypeptide is I53-50B (SEQ ID NO:8), I53-50B.1 (SEQ ID NO:32), I53-50B.1NegT2 (SEQ ID NO:33), or I53-50B.4PosT1 (SEQ ID NO:34); or
(d) when each fusion protein is DS-Cav1-I32-28A (SEQ ID NO:75), each second polypeptide is I32-28B (SEQ ID NO:22).

23. The nanostructure of claim 16, wherein each first polypeptide comprises a fusion protein of DS-Cav1 (SEQ ID NO:53) linked to SEQ ID NO:7 (I53-50A) via an amino acid linker.

24. The nanostructure of claim 23, wherein the amino acid linker comprises a Gly-Ser linker and/or a helical extension domain.

25. The nanostructure of claim 11, wherein each fusion protein comprises the amino acid sequence selected from the group consisting of SEQ ID NOS: 69-77 and 81-90.

26. The nanostructure of claim 23, wherein each fusion protein comprises the amino acid sequence of SEQ ID NO:76 (F10).

27. The nanostructure of claim 23, wherein each second polypeptide comprises the amino acid sequence selected from the group consisting of I53-50B (SEQ ID NO:8), I53-50B.1 (SEQ ID NO:32), I53-50B.1NegT2 (SEQ ID NO:33), or I53-50B.4PosT1 (SEQ ID NO:34).

28. The nanostructure of claim 23, wherein each second polypeptide comprises the amino acid sequence of I53-50B.4PosT1 (SEQ ID NO:34).

29. The nanostructure of claim 1, wherein the nanostructure:
(a) binds prefusion F-specific antibodies including but not limited to monoclonal antibody 25;
(b) forms a symmetrical structure, including but not limited to an icosahedral structure;
(c) is stable at 50° C.; and/or
(d) is stable in 2.25M guanidine hydrochloride.

30. A recombinant nucleic acid encoding the first polypeptide fusion protein as recited in claim 11.

31. A recombinant expression vector comprising the recombinant nucleic acid of claim 30 operatively linked to a promoter.

32. A recombinant host cell, comprising the recombinant expression vector of claim 31.

33. A recombinant host cell, comprising one or more recombinant expression vectors capable of expressing the first polypeptides and the second polypeptides of claim 1.

34. An immunogenic composition comprising the nanostructure of claim 1, and a pharmaceutically acceptable carrier.

35. The immunogenic composition of claim 34, further comprising an adjuvant.

36. A method for generating an immune response to paramyxovirus and/or pneumovirus F protein in a subject, comprising administering to the subject in need thereof an effective amount of the nanostructure or immunogenic composition of claim 1 to generate the immune response.

37. A method for treating or limiting a paramyxovirus and/or pneumovirus infection in a subject, comprising administering to the subject in need thereof an effective amount of the nanostructure or immunogenic composition of claim 1, thereby treating or preventing paramyxovirus and/or pneumovirus infection in the subject.

38. The method of claim 36, wherein the administering results in production of paramyxovirus and/or pneumovirus neutralizing antibodies in the subject.

39. The method of claim 38, wherein the neutralizing antibodies are present in sera of the subject at a titer (1/ID₅₀) of at least 9,400.

40. A process for assembling the nanostructures of claim 1 in vitro, comprising mixing two or more nanostructure components in aqueous conditions to drive spontaneous assembly of the desired nanostructure.

41. The process of claim 40, wherein the mixing comprises mixing first assemblies comprising first polypeptides (such as trimeric first polypeptides) each comprising an F protein or antigenic fragment thereof (“F protein”) with appropriate second assemblies comprising second polypeptides in an approximately 1:1 molar first polypeptide: second polypeptide ratio under conditions and for a time suitable to permit interaction of the first assemblies and the second assemblies to form the nanostructure.

42. The process of claim 40, wherein the mixing comprises mixing first assemblies comprising first polypeptides (such as trimeric first polypeptides), wherein fewer than all first polypeptides (for example, 75%, 50%, 25%, etc.) comprise an F protein with appropriate second assemblies comprising second polypeptides in an approximately 1:1 first polypeptide: second polypeptide molar ratio under conditions and for a time suitable to permit interaction of the first assemblies and the second assemblies to form the nanostructure.

43. The process of claim 40, wherein the mixing comprises mixing first assemblies comprising first polypeptides (such as trimeric first polypeptides) each comprising an F protein, wherein in total the first polypeptides comprise multiple different F proteins (for example, 2, 3, 4, or more) with appropriate second assemblies comprising second polypeptides in an approximately 1:1 molar first polypeptide: second polypeptide ratio under conditions and for a time suitable to permit interaction of the first assemblies and the second assemblies to form the nanostructure comprising multiple F proteins, or antigenic fragments thereof.
